(19) United States
(12) Patent Application Publication
(10) Pub. No.: US 2016/0201099 A1
(43) Pub. Date: Jul. 14, 2016

(54) CHIMERIC NON-RIBOSOMAL PEPTIDE SYNTHETASE

(71) Applicant: Dow AgroSciences LLC, Indianapolis, IN (US)

(72) Inventors: Amudhan VENKATESWARAN, Indianapolis, IN (US); Babu RAMAN, Indianapolis, IN (US); Paul SWANSON, Indianapolis, IN (US); Paul LEWER, Indianapolis, IN (US)

(21) Appl. No.: 14/865,734

(22) Filed: Sep. 25, 2015

Related U.S. Application Data
(60) Provisional application No. 62/056,213, filed on Sep. 26, 2014.

(51) Int. Cl. C12P 13/04; C12N 15/52; C12N 9/16; C12N 9/28; C07K 14/32; C12N 9/00

(52) U.S. Cl. CPC C12P 13/04; C07K 14/32; C12N 9/93; C12N 9/16; C12N 9/2417; C12N 15/52; C12Y 301/02; C12Y 603/02; C12Y 302/01001

(57) ABSTRACT

The present disclosure provides novel compositions and methods for the production and use of polynucleotide sequences encoding a chimeric non-ribosomal peptide synthetase (NRPS) fusion protein for the biosynthesis of N-acylglycine biosurfactants within a heterologous expression system.

### CROSS-REFERENCE TO RELATED APPLICATIONS

This application claims the benefit under 35 USC §119(e) of U.S. Provisional Application Ser. No. 62/056,213, filed on Sep. 26, 2014, the entire disclosure of which is incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 650,665 byte ASCII (Text) file named “14764-241762_SL.txt” created on Sep. 25, 2015.

### BACKGROUND OF THE INVENTION

N-acylglycine surfactants have traditionally been synthesized via chemical manufacturing processes that utilize chemical feedstocks. The production and manufacture of such surfactants rely upon the use of petrochemicals that are a non-renewable energy source. As such, the costs associated with obtaining petrochemical feedstocks fluctuate with the economic markets. N-acylglycine surfactants must be synthesized via complex chemical processes that require numerous steps of distinct and separate chemical reactions. Finally, the traditional manufacturing process of N-acylglycine surfactants produce chemical waste products that must be remediated for proper disposal.

Therefore, a need exists for development of improved synthesis and manufacturing processes of N-acylglycine surfactants acids via renewable production systems such as microbial fermentation.

The foregoing examples of the related art and limitations related therewith are intended to be illustrative and not exclusive. Other limitations of the related art will become apparent to those of skill in the art upon a reading of the specification.

### BRIEF SUMMARY OF THE INVENTION

The present disclosure is directed to a metabolically-engineered microorganism capable of synthesizing an N-acylglycine biosurfactant, the microorganism comprising a comprising a chimeric fusion protein. Generally, the chimeric fusion protein comprises a glycine adenylation domain operably linked to a condensation domain, a peptidyl carrier protein domain, a thioesterase domain, and the type II TE domain. In some embodiments glycine adenylation domain is isolated from a DhbF protein of SEQ ID NO:10 or a PksJ protein of SEQ ID NO:12. In other embodiments, the glycine adenylation domain protein motif comprises is a polypeptide of SEQ ID NO:40, SEQ ID NO:41, SEQ ID NO:42, SEQ ID NO:89, or SEQ ID NO:1. In further embodiments, the glycine adenylation domain comprises from the DhbF protein of SEQ ID NO:9 or the PksJ protein of SEQ ID NO:11. Further embodiments, include the condensation domain, the peptidyl carrier protein domain, the thioesterase domain, and the type II TE domain that are encoded by the surfactin gene cluster. The surfactin gene cluster can be selected from SrfAA-M3 (SEQ ID NO:4), SrfAA-M2 (SEQ ID NO:3), SrfAA-M1 (SEQ ID NO:2), SrfAB-M6 (SEQ ID NO:7), SrfAB-M5 (SEQ ID NO:6), SrfAB-M4 (SEQ ID NO:5), of SrfAC-M7 (SEQ ID NO:8).

In another aspect of the subject disclosure, the glycine adenylation domain is operably linked to the condensation domain at the amino acid sequence of SDAEKQM (SEQ ID NO: 53) or TLISDAEK (SEQ ID NO: 54), wherein E comprises the junction between the glycine adenylation domain and the condensation domain. In another embodiment, the glycine adenylation domain is operably linked to the peptidyl carrier protein domain at the amino acid sequence of WQEVLNVEKAGIF (SEQ ID NO: 57), wherein N comprises the junction between the glycine adenylation domain and the peptidyl carrier protein domain. In a further embodiment, the glycine adenylation domain is operably linked to the peptidyl carrier protein domain at the amino acid sequence of RVGIDDDFFALG (SEQ ID NO: 56) or IEWDDDFFAL (SEQ ID NO: 55), wherein the third D comprises the junction between the glycine adenylation domain and the peptidyl carrier protein domain.

In one aspect the microorganism of the subject disclosure is a gram (−) or a gram (+) bacteria. Exemplary gram (+) bacterium can be Bacillus subtilis. Exemplary gram (−) bacteria can be Escherichia coli. In other aspects of the disclosure, a polynucleotide encoding the chimeric fusion protein is expressed by a bacterial promoter. An exemplary bacterial promoter can be a PsrfA bacterial promoter. In another aspect of the disclosure, the polynucleotide encoding the chimeric fusion protein is codon optimized for expression in the microorganism. In a further aspect of the subject disclosure, the polynucleotide encoding the chimeric fusion protein is integrated within a genomic locus of the microorganism. An exemplary genomic locus can be the amyE genomic locus of a microorganism. In an embodiment, the integration within the genomic locus of a microorganism occurs via homologous recombination. In another aspect of the subject disclosure, the polynucleotide encoding the chimeric fusion protein is integrated within an autonomously replicating plasmid. In other embodiments, the chimeric fusion protein is encoded by a polynucleotide with at least 90% sequence identity to ME-B0004 (SEQ ID NO:15), ME-B0007 (SEQ ID NO:13), and ME-B0008 (SEQ ID NO:14). The subject disclosure herein relates to a metabolically-engineered microorganism that expresses a chimeric fusion protein that subsequently results in the synthesis of N-acylglycine from medium chain length β-hydroxy fatty acids.

The present disclosure is further directed to a method for producing N-acylglycine from a microorganism. The microorganism comprising a polynucleotide encoding a chimeric fusion protein is provided. The microorganism is cultured to produce a medium chain length β-hydroxy fatty acid. The chimeric fusion protein is expressed, wherein the expression of the chimeric fusion protein synthesizes N-acylglycine from the medium chain length β-hydroxy fatty acid. N-acylglycine is purified from the microorganism.

The present disclosure is directed to a method for fermenting N-acylglycine within a microorganism. The microorganism comprising a polynucleotide encoding a chimeric fusion protein in is fermented. The chimeric fusion protein is expressed, wherein the expression of the chimeric fusion protein synthesizes N-acylglycine from a medium chain length β-hydroxy fatty acid. N-acylglycine is fermented within the microorganism.

In another aspect, the present disclosure is directed to a chimeric polynucleotide sequence comprising a glycine adenylation domain. In an embodiment, the glycine adenylation domain is selected from: a polynucleotide motif comprising a glycine adenylation domain motif of SEQ ID NO:40, SEQ ID NO:41, SEQ ID NO:42, or SEQ ID NO:1; a polynucleotide comprising at least 90% sequence identity to the polynucleotide of SEQ ID NO:49, or SEQ ID NO:52; a polynucleotide encoding a polypeptide comprising at least 90% sequence identity to SEQ ID NO:10, SEQ ID NO:12, SEQ ID NO:58, SEQ ID NO:59, SEQ ID NO:60, SEQ ID NO:61, SEQ ID NO:62, SEQ ID NO:63, SEQ ID NO:64, SEQ ID NO:65, or SEQ ID NO:66; and, a polynucleotide of a coding sequence comprising a glycine adenylation domain of Table 1. In further embodiments, the glycine adenylation domain is operably linked to a condensation domain. An exemplary condensation domain comprises a polynucleotide with at least 90% sequence identity to SEQ ID NO:50. Further examples of a condensation domain include a polynucleotide encoding a polypeptide with at least 90% sequence identity to SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76. In another embodiment, the glycine adenylation domain is operably linked to a peptidyl carrier protein domain. An exemplary peptidyl carrier protein domain comprises a polynucleotide with at least 90% sequence identity to SEQ ID NO:51. Further examples of a peptidyl carrier protein domain include a polynucleotide encoding a polypeptide with at least 90% sequence identity to SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, or SEQ ID NO:86. In an additional embodiment, the glycine adenylation domain is operably linked to a thioesterase domain. An exemplary thioesterase domain comprises a polynucleotide with at least 90% sequence identity to SEQ ID NO:51. Further examples of a thioesterase domain include a polypeptide with at least 90% sequence identity to SEQ ID NO:87, or SEQ ID NO:88. In an embodiment, the chimeric polynucleotide sequence comprises a NRPS fusion gene construct of a polypeptide with at least 90% sequence identity to SEQ ID NO:13, SEQ ID NO:14, or SEQ ID NO:15. The expression of the NRPS fusion gene construct results in the synthesis of N-acylglycine from a medium chain length β-hydroxy fatty acid. In further embodiments, the glycine adenylation domain of the chimeric polynucleotide sequence is transformed into a bacterial microorganism. The resulting bacterial microorganism results in synthesis of an N-acylglycine biosurfactant from a medium chain length β-hydroxy fatty acid.

In addition to the exemplary aspects and embodiments described above, further aspects and embodiments will become apparent by study of the following descriptions.

### BRIEF DESCRIPTION OF THE FIGURES

FIG. 1 illustrates an overview of the NRPS domain construction strategy for the fusion construct. The following domains are provided; C: condensation domain, A: adenylation domain, P: peptidyl carrier protein domain, and TE: thioesterase domain, SrfAD; typeII-thioesterase.

FIG. 2 illustrates the ClustalW2® alignment of protein sequences of C and A domain junctions, and the A and PCP domain junctions. The amino-acid residues highlighted and identified with an asterisk were identified as the fusion points between different domains. The amino-acid residues highlighted define the junction between different domains as identified by the NRPS predictor software. FIG. 2A discloses SEQ ID NOS 97-105, respectively, in order of appearance. FIG. 2B discloses SEQ ID NOS 106-115, respectively, in order of appearance.

FIG. 3A illustrates the module fusion strategy for construct ME-B007 (BSU3a) The highlighted amino-acid residues were the sequences used for the chimeric fusion and identify the fusion points between the domains of the NRPS fusion protein. FIG. 3A discloses SEQ ID NOS 101, 100, 97, 104, 99, 98, 105, 102, 103, 111, 108, 112, 110, 114, 113, 109, 106, and 115, respectively, in order of appearance.

FIG. 3B illustrates the module fusion strategy for construct ME-B008 (BSU4a) The highlighted amino-acid residues were the sequences used for the chimeric fusion and identify the fusion points between the domains of the NRPS fusion protein. FIG. 3B discloses SEQ ID NOS 101, 100, 97, 104, 99, 98, 105, 102, 103, 111, 108, 112, 110, 114, 113, 109, 106, and 115, respectively, in order of appearance.

FIG. 3C illustrates the module fusion strategy for construct ME-B004 (Dhbf3a) The highlighted amino-acid residues were the sequences used for the chimeric fusion and identify the fusion points between the domains of the NRPS fusion protein. FIG. 3C discloses SEQ ID NOS 101, 100, 97, 104, 99, 98, 105, 102, 103, 111, 108, 112, 110, 114, 113, 109, 106, and 115, respectively, in order of appearance.

FIG. 4 illustrates the plasmid map of the fusion gene expression construct of ME-B007.

FIG. 5 illustrates the plasmid map of the fusion gene expression construct of ME-B008.

FIG. 6 illustrates the plasmid map of the fusion gene expression construct of ME-B004.

FIG. 7 illustrates an overview of the plasmid design of the NRPS fusion gene constructs and their genomic integration and expression in B. subtilis str. OKB120.

FIG. 8 illustrates a summary of structures referred to in Example 9, including two isomeric target N-acylglycine surfactant products (1) and (2) produced by the expression of the NRPS chimeric fusion proteins and an analytical standard (3).

FIG. 9A exemplifies LC-SIM-MS chromatograms for B. subtilis str. OKB120 engineered strains (OKB120-dhfb3) of ME-B0004 expressing NRPS fusion protein containing glycine specific adenylation domain from the non-native B. subtilis protein dhbF, which were used to quantify the products 1 and 2.

FIG. 9B exemplifies LC-SIM-MS chromatograms for B. subtilis str. OKB120 engineered strains (OKB120-BSU3a) expressing NRPS fusion protein containing glycine specific adenylation domain from the non-native B. subtilis protein BSU17180 (PksJ), which were used to quantify the products 1 and 2.

FIG. 9C exemplifies LC-SIM-MS chromatograms for B. subtilis str. OKB120 engineered strains (OKB120-BSU4a) expressing NRPS fusion protein containing glycine specific adenylation domain from the non-native B. subtilis protein BSU17180 (PksJ), which were used to quantify the products 1 and 2.

FIG. 10 provides a CLUSTAL 2.1® multiple sequence alignment of the five glycine-specific adenylation domains from Bacillus subtilis, Bacillus amyloliquefaciens, and Streptomyces roseosporus. The amino acids lining the substrate binding pocket of the adenylation domain which confer specificity (corresponding to amino acid residues at positions 235, 236, 239, 278, 299, 301, 322, 330, and 331 of the substrate-binding pocket of the gramicidin S synthase (GrsA) phenylalanine activating domain; Challis G L, Ravel J, Townsend C A. Predictive, structure-based model of amino acid recognition by nonribosomal peptide synthetase adenylation domains. Chem Biol. 2000 March; 7(3):211-24, and Stachelhaus T, Mootz H D, Marahiel M A. The specificity-conferring code of adenylation domains in nonribosomal peptide synthetases. Chem Biol. 1999 August; 6(8):493-505) are boxed.

### DETAILED DESCRIPTION

### I. Overview

Disclosed herein are novel, chimeric nonribosomal peptide synthetase (NRPS) fusion protein sequences that result from the engineering of a glycine-specific adenylation domain within the NRPS fusion protein. The NRPS is a modular multi-domain enzyme that can selectively bind and condense amino acids to enzymatically produce products of varied structural and functional diversity. The number of modules and their organization comprising the NRPS determines the primary structure of the corresponding peptide products. A component of each module of the NRPS machinery is an ‘adenylation domain (herein referred to as an “A domain” or “A-domain”)’ that recognizes a specific amino acid building block and recruits the amino acid to a peptidyl or amino-acyl chain. By exchanging the native A-domain for a glycine specific A-domain, the resulting chimeric NRPS fusion protein can be designed to specifically recognize the amino acid glycine. The chimeric NRPS fusion protein successfully enables the in vivo acylation of the amino acid glycine into a medium chain-length β-hydroxy fatty acid peptide chain. As such, the chimeric NRPS fusion protein is expressed in a prokaryotic species, and subsequently fermented to result in the production of the non-native lipoamino acid, N-acylglycine biosurfactant.

### II. Terms

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises”, “comprising”, “includes”, “including”, “has”, “having”, “contains”, or “containing”, or, any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “invention” or “present invention” as used herein is a non-limiting term and is not intended to refer to any single embodiment of the particular invention but encompasses all possible embodiments as disclosed in the application.

As used herein, “endogenous sequence” defines the native form of a polynucleotide, gene or polypeptide in its natural location in the organism or in the genome of an organism.

The term “isolated”, as used herein means having been removed from its natural environment.

The term “purified”, as used herein relates to the isolation of a molecule or compound in a form that is substantially free of contaminants normally associated with the molecule or compound in a native or natural environment and means having been increased in purity as a result of being separated from other components of the original composition. The term “purified nucleic acid” is used herein to describe a nucleic acid sequence which has been separated from other compounds including, but not limited to polypeptides, lipids and carbohydrates.

As used herein, the terms “polynucleotide”, “nucleic acid”, and “nucleic acid molecule” are used interchangeably, and may encompass a singular nucleic acid; plural nucleic acids; a nucleic acid fragment, variant, or derivative thereof; and nucleic acid construct (e.g., messenger RNA (mRNA) and plasmid DNA (pDNA)). A polynucleotide or nucleic acid may contain the nucleotide sequence of a full-length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single- and double-stranded regions; single- and double-stranded RNA; and RNA that is mixture of single- and double-stranded regions. Hybrid molecules comprising DNA and RNA may be single-stranded, double-stranded, or a mixture of single- and double-stranded regions. The foregoing terms also include chemically, enzymatically, and metabolically modified forms of a polynucleotide or nucleic acid.

It is understood that a specific DNA or polynucleotide refers also to the complement thereof, the sequence of which is determined according to the rules of deoxyribonucleotide base-pairing. Although only one strand of DNA may be presented in the sequence listings of this disclosure, those having ordinary skill in the art will recognize that the complementary strand can be ascertained and determined from the strand presented herein. Accordingly, a single strand of a polynucleotide can be used to determine the complementary strand, and, accordingly, both strands (i.e., the sense strand and anti-sense strand) are exemplified from a single strand.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product.

As used herein, the term “coding sequence” refers to a nucleic acid sequence that encodes a specific amino acid sequence. A “regulatory sequence” refers to a nucleotide sequence located upstream (e.g., 5′ non-coding sequences), within, or downstream (e.g., 3′ non-coding sequences) of a coding sequence, which influence the transcription, RNA processing or stability, or translation of the associated coding sequence. Regulatory sequences include, for example and without limitation: promoters; translation leader sequences; introns; polyadenylation recognition sequences; RNA processing sites; effector binding sites; and stem-loop structures.

As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of “polypeptide”, and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis. Likewise, a polypeptide may be generated by expressing a native coding sequence, or portion thereof, that is introduced into an organism in a form that is different from the corresponding native coding sequence.

In contrast, the term “heterologous” refers to a polynucleotide, gene or polypeptide that is not normally found at its location in the reference (host) organism. For example, a heterologous nucleic acid may be a nucleic acid that is normally found in the reference organism at a different genomic location. By way of further example, a heterologous nucleic acid may be a nucleic acid that is not normally found in the reference organism. A host organism comprising a hetereologous polynucleotide, gene or polypeptide may be produced by introducing the heterologous polynucleotide, gene or polypeptide into the host organism. In particular examples, a heterologous polynucleotide comprises a native coding sequence, or portion thereof, that is reintroduced into a source organism in a form that is different from the corresponding native polynucleotide. In particular examples, a heterologous gene comprises a native coding sequence, or portion thereof, that is reintroduced into a source organism in a form that is different from the corresponding native gene. For example, a heterologous gene may include a native coding sequence that is a portion of a chimeric gene including non-native regulatory regions that is reintroduced into the native host. In particular examples, a heterologous polypeptide is a native polypeptide that is reintroduced into a source organism in a form that is different from the corresponding native polypeptide.

A heterologous, gene or polypeptide may be a gene or polypeptide that comprises a functional polypeptide or nucleic acid sequence encoding a functional polypeptide that is fused to another gene or polypeptide to produce a chimeric or fusion polypeptide, or a gene encoding the same. Genes and proteins of particular embodiments include specifically exemplified full-length sequences and portions, segments, fragments (including contiguous fragments and internal and/or terminal deletions compared to the full-length molecules), variants, mutants, chimerics, and fusions of these sequences.

As used herein, the term “modification” can refer to a change in a polynucleotide disclosed herein that results in reduced, substantially eliminated or eliminated activity of a polypeptide encoded by the polynucleotide, as well as a change in a polypeptide disclosed herein that results in reduced, substantially eliminated or eliminated activity of the polypeptide. Alternatively, the term “modification” can refer to a change in a polynucleotide disclosed herein that results in increased or enhanced activity of a polypeptide encoded by the polynucleotide, as well as a change in a polypeptide disclosed herein that results in increased or enhanced activity of the polypeptide. Such changes can be made by methods well known in the art, including, but not limited to, deleting, mutating (e.g., spontaneous mutagenesis, random mutagenesis, mutagenesis caused by mutator genes, or transposon mutagenesis), substituting, inserting, down-regulating, altering the cellular location, altering the state of the polynucleotide or polypeptide (e.g., methylation, phosphorylation or ubiquitination), removing a cofactor, introduction of an antisense RNA/DNA, introduction of an interfering RNA/DNA, chemical modification, covalent modification, irradiation with UV or X-rays, homologous recombination, mitotic recombination, promoter replacement methods, and/or combinations thereof. Guidance in determining which nucleotides or amino acid residues can be modified, can be found by comparing the sequence of the particular polynucleotide or polypeptide with that of homologous polynucleotides or polypeptides, e.g., yeast or bacterial, and maximizing the number of modifications made in regions of high homology (conserved regions) or consensus sequences.

The term “derivative”, as used herein, refers to a modification of a sequence set forth in the present disclosure. Illustrative of such modifications would be the substitution, insertion, and/or deletion of one or more bases relating to a nucleic acid sequence of a coding sequence disclosed herein that preserve, slightly alter, or increase the function of a coding sequence disclosed herein in crop species. Such derivatives can be readily determined by one skilled in the art, for example, using computer modeling techniques for predicting and optimizing sequence structure. The term “derivative” thus also includes nucleic acid sequences having substantial sequence identity with the disclosed coding sequences herein such that they are able to have the disclosed functionalities for use in producing embodiments of the present disclosure.

The term “promoter” refers to a DNA sequence capable of controlling the expression of a nucleic acid coding sequence or functional RNA. In examples, the controlled coding sequence is located 3′ to a promoter sequence. A promoter may be derived in its entirety from a native gene, a promoter may be comprised of different elements derived from different promoters found in nature, or a promoter may even comprise rationally designed DNA segments. It is understood by those skilled in the art that different promoters can direct the expression of a gene in different cell types, or at different stages of development, or in response to different environmental or physiological conditions. Examples of all of the foregoing promoters are known and used in the art to control the expression of heterologous nucleic acids. Promoters that direct the expression of a gene in most cell types at most times are commonly referred to as “constitutive promoters.” Furthermore, while those in the art have (in many cases unsuccessfully) attempted to delineate the exact boundaries of regulatory sequences, it has come to be understood that DNA fragments of different lengths may have identical promoter activity. The promoter activity of a particular nucleic acid may be assayed using techniques familiar to those in the art.

The term “operably linked” refers to an association of nucleic acid sequences on a single nucleic acid, wherein the function of one of the nucleic acid sequences is affected by another. For example, a promoter is operably linked with a coding sequence when the promoter is capable of effecting the expression of that coding sequence (e.g., the coding sequence is under the transcriptional control of the promoter). A coding sequence may be operably linked to a regulatory sequence in a sense or antisense orientation.

The term “expression”, as used herein, may refer to the transcription and stable accumulation of sense (mRNA) or antisense RNA derived from a DNA. Expression may also refer to translation of mRNA into a polypeptide. As used herein, the term “overexpression” refers to expression that is higher than endogenous expression of the same gene or a related gene. Thus, a heterologous gene is “overexpressed” if its expression is higher than that of a comparable endogenous gene.

As used herein, the term “transformation” or “transforming” refers to the transfer and integration of a nucleic acid or fragment thereof into a host organism, resulting in genetically stable inheritance. Host organisms containing a transforming nucleic acid are referred to as “transgenic,” “recombinant,” or “transformed” organisms.

The terms “plasmid” and “vector”, as used herein, refer to an extra chromosomal element that may carry one or more gene(s) that are not part of the central metabolism of the cell. Plasmids and vectors typically are circular double-stranded DNA molecules. However, plasmids and vectors may be linear or circular nucleic acids, of a single- or double-stranded DNA or RNA, and may carry DNA derived from essentially any source, in which a number of nucleotide sequences have been joined or recombined into a unique construction that is capable of introducing a promoter fragment and a coding DNA sequence along with any appropriate 3′ untranslated sequence into a cell. In examples, plasmids and vectors may comprise autonomously replicating sequences for propagation in bacterial hosts.

“Polypeptide” and “protein” are used interchangeably herein and include a molecular chain of two or more amino acids linked through peptide bonds. The terms do not refer to a specific length of the product. Thus, “peptides”, and “oligopeptides”, are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be synthesized, or expressed recombinantly using known protein engineering techniques. In addition, inventive fusion proteins can be derivatized as described herein by well-known organic chemistry techniques.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

Expression “control sequences” refers collectively to promoter sequences, ribosome binding sites, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Recombination” refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

The terms “stringent conditions” or “hybridization under stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and Northern hybridizations are sequence dependent, and produce different results under varying experimental parameters. An extensive guide to the hybridization of nucleic acids is found in Tijssen (1993) Laboratory Techniques in Biochemistry and Molecular Biology—Hybridization with Nucleic Acid Probes, Part I, Chapter 2: Overview of principles of hybridization and the strategy of nucleic acid probe assays, Elsevier, New York. Generally, “highly stringent conditions” result in the hybridization of a probe to a polynucleotide sequence, wherein the probe and polynucleotide sequence share at least 85% sequence identity. The “highly stringent conditions” include stringent hybridization and wash conditions that are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. “Very highly stringent conditions” result in the hybridization of a probe to a polynucleotide sequence, wherein the probe and polynucleotide sequence share at least 95% sequence identity. The “very highly stringent conditions” include stringent hybridization and wash conditions that are selected to be equal to the Tm for a particular probe.

An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The disclosure also relates to a polynucleotide probe hybridizable under stringent conditions, and in some instances under highly stringent conditions, and in further instances under very highly stringent conditions to a polynucleotide as of the present disclosure.

As used herein, the term “hybridizing” is intended to describe conditions for hybridization and washing under “stringent conditions” for which nucleotide sequences at least about 50%, at least about 60%, at least about 70%, more preferably at least about 80% identical to each other typically remain hybridized to each other. As used herein, the term “hybridizing” is intended to describe conditions for hybridization and washing under “highly stringent conditions” for which nucleotide sequences at least about 85%, at least about 90%, identical to each other typically remain hybridized to each other. As used herein, the term “hybridizing” is intended to describe conditions for hybridization and washing under “very highly stringent conditions” for which nucleotide sequences at least about 95%, at least about 99%, identical to each other typically remain hybridized to each other.

In some embodiments an isolated nucleic acid molecule of the disclosure that hybridizes under highly stringent conditions to a nucleotide sequence of the disclosure can correspond to a naturally-occurring nucleic acid molecule. As used herein, a “naturally-occurring” nucleic acid molecule refers to an RNA or DNA molecule having a nucleotide sequence that occurs in nature (e.g., encodes a natural protein).

A skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

The terms “homology” or “percent identity” are used interchangeably herein. For the purpose of this disclosure, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available on the internet at the accelrys website, more specifically at http://www.accelrys.com), using either a Blossom 62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available on the internet at the accelrys website, more specifically at http://www.accelrys.com), using a NWSgapdna.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (CABIOS, 4: 11-17 (1989) which has been incorporated into the ALIGN program (version 2.0) (available on the internet at the vega website, more specifically ALIGN-IGH Montpellier, or more specifically at http://vega.igh.cnrs.fr/bin/align-guess.cgi) using a PAM120 weight residue table, a gap length penalty of 12 and a gap penalty of 4.

The nucleic acid and protein sequences of the present disclosure may further be used as a “query sequence” to perform a search against public databases to, for example, identify other family members or related sequences. Such searches may be performed using the BLASTN and BLASTX programs (version 2.0) of Altschul, et al. (1990) J. Mol. Biol. 215:403-10. BLAST nucleotide searches may be performed with the BLASTN program, score=100, word length=12 to obtain nucleotide sequences identical to the nucleic acid molecules of the present disclosure. BLAST protein searches may be performed with the BLASTX program, score=50, word length=3 to obtain amino acid sequences identical to the protein molecules of the present disclosure. To obtain gapped alignments for comparison purposes, Gapped BLAST may be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) may be used. (Available on the internet at the ncbi website, more specifically at www.ncbi.nlm.nih.gov).

The term “motif” refers to short regions of conserved sequences of nucleic acids or amino acids that comprise part of a longer sequence.

The term “variant” refers to substantially similar sequences. Generally, nucleic acid sequence variants of the invention will have at least 46%, 48%, 50%, 52%, 53%, 55%, 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98% or 99% sequence identity to the native nucleotide sequence, wherein the % sequence identity is based on the entire sequence and is determined by GAP 10 analysis using default parameters. Generally, polypeptide sequence variants of the invention will have at least about 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the native protein, wherein the % sequence identity is based on the entire sequence and is determined by GAP 10 analysis using default parameters. GAP uses the algorithm of Needleman and Wunsch (J. MoI. Biol. 48:443-453, 1970) to find the alignment of two complete sequences that maximizes the number of matches and minimizes the number of gaps.

The term “variant” also refers to substantially similar sequences that contain amino acid sequences highly similar to the motifs contained within the invention and optionally required for the biological function of the invention. Generally, polypeptide sequence variants of the invention will have at least 85%, 90% or 95% sequence identity to the conserved amino acid residues in the defined motifs.

Variants included in the invention may contain individual substitutions, deletions or additions to the nucleic acid or polypeptide sequences which alter, add or delete a single amino acid or a small percentage of amino acids in the encoded sequence. A “conservatively modified variant” is an alteration which results in the substitution of an amino acid with a chemically similar amino acid. When the nucleic acid is prepared or altered synthetically, advantage can be taken of known codon preferences of the intended host. The nucleic acid fragments of the instant invention may be used to isolate cDNAs and genes encoding proteins with homology or sequence identity from the same or other species. Isolation of homologous genes or genes with levels of shared sequence identity using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other NRPS fusion proteins, either as cDNAs or genomic DNAs, could be isolated directly by using all or a portion of the instant nucleic acid fragments as DNA hybridization probes to screen libraries from any desired organism employing methodology well known to those skilled in the art. Specific oligonucleotide probes based upon the instant nucleic acid sequences can be designed and synthesized by methods known in the art (Sambrook). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

Strategies for designing and constructing variant genes and proteins that comprise contiguous residues of a particular molecule can be determined by obtaining and examining the structure of a protein of interest (e.g., atomic 3-D (three dimensional) coordinates from a crystal structure and/or a molecular model). In some examples, a strategy may be directed to certain segments of a protein that are ideal for modification, such as surface-exposed segments, and not internal segments that are involved with protein folding and essential 3-D structural integrity. U.S. Pat. No. 5,605,793, for example, relates to methods for generating additional molecular diversity by using DNA reassembly after random or focused fragmentation. This can be referred to as gene “shuffling”, which typically involves mixing fragments (of a desired size) of two or more different DNA molecules, followed by repeated rounds of renaturation. This process may improve the activity of a protein encoded by a subject gene. The result may be a chimeric protein having improved activity, altered substrate specificity, increased enzyme stability, altered stereospecificity, or other characteristics.

An amino acid “substitution” can be the result of replacing one amino acid in a reference sequence with another amino acid having similar structural and/or chemical properties (i.e., conservative amino acid substitution), or it can be the result of replacing one amino acid in a reference sequence with an amino acid having different structural and/or chemical properties (i.e., non-conservative amino acid substitution). Amino acids can be placed in the following structural and/or chemical classes: non-polar; uncharged polar; basic; and acidic. Accordingly, “conservative” amino acid substitutions can be made on the basis of similarity in polarity, charge, solubility, hydrophobicity, hydrophilicity, or the amphipathic nature of the residues involved. For example, non-polar (hydrophobic) amino acids include glycine, alanine, leucine, isoleucine, valine, proline, phenylalanine, tryptophan, and methionine; uncharged (neutral) polar amino acids include serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in the polarity, charge, solubility, hydrophobicity, hydrophilicity, or amphipathic nature of any of these amino acids. “Insertions” or “deletions” can be within the range of variation as structurally or functionally tolerated by the recombinant proteins.

In some embodiments, a variant protein is “truncated” with respect to a reference, full-length protein. In some examples, a truncated protein retains the functional activity of the reference protein. By “truncated” protein, it is meant that a portion of a protein may be cleaved off, for example, while the remaining truncated protein retains and exhibits the desired activity after cleavage. Cleavage may be achieved by any of various proteases. Furthermore, effectively cleaved proteins can be produced using molecular biology techniques, wherein the DNA bases encoding a portion of the protein are removed from the coding sequence, either through digestion with restriction endonucleases or other techniques available to the skilled artisan. A truncated protein may be expressed in a heterologous system, for example, B. subtilis, E. coli, baculoviruses, plant-based viral systems, and yeast. Truncated proteins conferring nonribosomal peptide synthetase activity may be confirmed by using the heterologous expression system expressing the proteins, such as described herein. It is well-known in the art that truncated proteins can be successfully produced so that they retain the functional activity of the full-length reference protein. For example, Bt proteins can be used in a truncated (core protein) form. See, e.g., Hofte and Whiteley (1989) Microbiol. Rev. 53(2):242-55; and Adang et al. (1985) Gene 36:289-300.

In some cases, especially for expression in bacterial strains, it can be advantageous to use truncated genes that express truncated proteins. Truncated genes may encode a polypeptide comprised of, for example, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full-length protein. The variant genes and proteins that retain the function of the reference sequence from which they were designed may be determined by one of skill in the art, for example, by assaying recombinant variants for activity. If such an activity assay is known and characterized, then the determination of functional variants requires only routine experimentation.

Specific changes to the “active site” of an enzyme may be made to affect the inherent functionality with respect to activity or stereospecificity. See, Muller et. al. (2006) Protein Sci. 15(6): 1356-68. For example, the known tauD structure has been used as a model dioxygenase to determine active site residues while bound to its inherent substrate, taurine. See, Elkins et al. (2002) Biochemistry 41(16):5185-92. Further information regarding sequence optimization and designability of enzyme active sites can be found in Chakrabarti et al. (2005) Proc. Natl. Acad. Sci. USA 102(34):12035-40.

Various structural properties and three-dimensional features of a protein may be changed without adversely affecting the activity/functionality of the protein. Conservative amino acid substitutions can be made that do not adversely affect the activity and/or three-dimensional configuration of the molecule (“tolerated” substitutions). Variant proteins can also be designed that differ at the sequence level from the reference protein, but which retain the same or similar overall essential three-dimensional structure, surface charge distribution, and the like. See, e.g., U.S. Pat. No. 7,058,515; Larson et al. (2002) Protein Sci. 1 1:2804-13; Crameri et al. (1997) Nat. Biotechnol. 15:436-8; Stemmer (1994) Proc. Natl. Acad. Sci. USA 91:10747-51; Stemmer (1994) Nature 370:389-91; Stemmer (1995) Bio/Technology 13:549-53; Crameri et al. (1996) Nat. Med. 2:100-3; and Crameri et al. (1996) Nat. Biotechnol. 14: 315-9.

The term “chimeric” as used herein, means comprised of sequences that are “recombined”. For example the sequences are recombined and are not found together in nature.

The term “recombine” or “recombination” as used herein means refers to any method of joining polynucleotides. The term includes end to end joining, and insertion of one sequence into another. The term is intended to encompass includes physical joining techniques such as sticky-end ligation and blunt-end ligation. Such sequences may also be artificially, or recombinantly synthesized to contain the recombined sequences. Additionally, the term can encompass the integration of one sequence within a second sequence, for example the integration of a polynucleotide within the genome of an organism by homologous recombination can result from “recombination”.

### III. Embodiments of the Present Disclosure

In an embodiment, the subject disclosure relates to prokaryotic microorganisms that are metabolically engineered to produce non-native lipoamino acid, N-acylglycine biosurfactants. Prokaryotic microorganisms can be utilized for production of novel compounds via fermentation in cultures. As such, the microorganism is metabolically-engineered via recombinant DNA technology for the production of the desired chemical compound. The subject disclosure describes a process to utilize recombinant DNA technology to design and build a novel, chimeric fusion protein for the production an N-acylglycine biosurfactant within a prokaryotic microorganism.

In certain embodiments the biosurfactant is a metabolic product produced by a microorganism. The biosurfactant molecules are composed of two distinct moieties: a hydrophilic and a hydrophobic moiety. Biosurfactants can be categorized as glycolipids (a carbohydrate linked to a fatty acid), proteolipids (an amino acid or chain of amino acids linked to a fatty acid), or polymeric surfactants (high molecular weight structures consisting of fatty acids). The metabolic product may be a fatty acid, and in some instances the surfactant is a beta-hydroxy fatty acid. Typically, the biosurfactant is biodegradable, less toxic, and produced more efficiently than synthetic compounds produced from chemical refinement of a feedstock (i.e., petrochemical feed stocks).

Various strains of microorganisms are capable of producing surfactants. For example; Bacillus sp. (i.e., Bacillus subtilis), Mycobacterium sp., Corynebacterium sp., Ustilago sp., Arthrobacter sp., Candida sp.; Pseudomonas sp., Torulopsis sp., Escherchia sp. and Rhodococcus sp. are only a few of the many various types of microorganisms that can naturally produce surfactants. In an embodiment, the metabolically engineered microorganism of the subject disclosure can comprise a Bacillus sp., Mycobacterium sp., Corynebacterium sp., Ustilago sp., Arthrobacter sp., Candida sp., Pseudomonas sp., Torulopsis sp., Escherchia sp., and Rhodococcus sp. In further embodiments, the metabolically engineered microorganism of the subject disclosure can comprise a yeast microorganism, a cyanobacterium microorganism, or a bacterial microorganism. Generally, bacterial microorganisms are categorized by differentiating bacterial species into gram positive or gram negative species. The gram staining is used to identify bacterial strains that contain peptidoglycan in the cell wall. This microbiological procedure is commonly known in the art, and would be appreciated as a common categorical process by those persons having ordinary skill in the art.

Heterologous expression of an enzyme and production of a biosurfactant in certain species of microorganisms can result in altered properties of the biosurfactant. For example, the chain length of the fatty acid of a biosurfactant may vary, in part, due to the microorganism that it is produced from. In addition, the fatty acid chain may be branched or contain additional chemical moieties (i.e., hydroxylation, acylation, alkylation, oxidation, etc.) thereby altering the chemical structure of the fatty acid moiety of a biosurfactant and further altering the functionality of the biosurfactant (i.e., length of fatty acid, charge, solubility in water, molecular weight, etc.). The microorganism from which the biosurfactant is produced will impart such properties on the biosurfactant. In certain embodiments of the subject disclosure the microorganism is engineered to acylate an amino acid (i.e., glycine) to a biosurfactant. In such embodiments, the microorganism is metabolically engineered to acylate the amino acid (i.e., glycine) to the biosurfactant.

In certain embodiments the amino acid, glycine, is acylated to a fatty acid (i.e., acyl-coA) to produce an N-acylglycine biosurfactant. In certain embodiments, the amino acid glycine is recruited into a medium chain-length β-hydroxy fatty acid peptide chain. “Beta-hydroxy fatty acids” are fatty acids (i.e., acyl-coA) comprising a hydroxy group at the third carbon (i.e., the beta position) of the fatty acid chain. Typically, the carboxylate moiety of the fatty acid is covalently attached to the nitrogen of the amino acid such that the beta position corresponds to the carbon two carbons removed from the carbon having the ester group. “Medium chain length” beta-hydroxy fatty acids may be in length of 3, 4, 5, 6, 7, 8, 9, 10, 11, 12, 3, 14, 15, 16, 17, 18, 19, 20, 21, 22, 23, 24, 25 or more carbon atoms. In some embodiments the amino acid glycine is linked to the beta-hydroxy fatty acids to produce an N-acylglycine surfactant in the length of 3, 4, 5, 6, 7, 8, 9, 10, 11, 12, 3, 14, 15, 16, 17, 18, 19, 20, 21, 22, 23, 24, 25 or more carbon atoms. In additional embodiments the amino acid glycine is covalently linked to the beta-hydroxy fatty acids to produce an N-acylglycine surfactant in the length of 3, 4, 5, 6, 7, 8, 9, 10, 11, 12, 3, 14, 15, 16, 17, 18, 19, 20, 21, 22, 23, 24, 25 or more carbon atoms.

In additional embodiments, the N-acylglycine surfactant may contain linear carbon chains, in which each carbon of the chain, with the exception of the terminal carbon atom and the carbon attached to the nitrogen of the amino acid, is directly covalently linked to two other carbon atoms. Additionally or alternatively, N-acylglycine surfactant may contain branched carbon chains, in which at least one carbon of the chain is directly covalently linked to three or more other carbon atoms. N-acylglycine surfactant may contain one or more double bonds between adjacent carbon atoms. Alternatively, N-acylglycine surfactant may contain only single-bonds between adjacent carbon atoms. Furthermore, different beta-hydroxy fatty acid linkage domains that exhibit specificity for other beta-hydroxy fatty acids (e.g., naturally or non-naturally occurring beta-hydroxy fatty acids) may be used to generate the N-acylglycine surfactant.

The fatty acid of a microorganism can vary, depending upon the bacterial strain, growth media, cultivation conditions, etc. Most bacteria produce straight-chain fatty acids with or without unsaturation in the carbon chain (myristic, palmitic, stearic, oleic, and linoleic acids). Branched-chain fatty acids with a methyl group at the penultimate (iso-) or the antepenultimate (anteiso-) positions are relatively uncommon but are the major constituents of lipids in gram positive bacteria such as Bacillus subtilis.

In B. subtilis, branched-chain fatty acids account for >90% of the total fatty acid pool (Roberts, 1994, IJSB, B. mojavensis—distinguishable from B. subtilis, V44(2), p. 256-264). Anteiso-fatty acids (anteiso-C15 and anteiso-C17 at about 40.19%±3.98% and 9.38%±0.95%, respectively) are the most abundant, with anteiso-C15 fatty acids being the single most abundant fatty acid in B. subtilis. The odd-numbered iso-fatty acids (iso-C15 and iso-C17 at about 29.27%±4.64% and 9.59%±1.56%, respectively) are next in order of abundance. The even-numbered iso-(iso-C14 and iso-C16 at about 1.13%±0.24% and 2.36%±0.34%, respectively) and straight-chain (n-C14 at about a concentration not currently measured and n-C16 at about 3.14%±0.40%) fatty acids are of relatively low abundance. Unsaturated fatty acids account for a small fraction of the lipid content in B. subtilis with C16:1 cis9, C16:1 cis5, and iso-C17:1 cis7 at about 0.23%±0.35%, 1.52%±0.45%, and 1.72% f 0.42%, respectively.

The observed trend in fatty composition in B. subtilis is also generally conserved across other species within the Bacillus genus such as B. alvei, B. amyloliquefaciens, B. atrophaeus, B. brevis, B. circulans, B. licheniformis, B. macerans, B. megaterium and B. pumilus (Kaneda, 1967, J. Bac., Fatty acids in the Genus Bacillus: Iso- and anteiso-fatty acids as characteristic constituents, V93(3), p. 894-903). Anteiso-fatty acids (anteiso-C15 and anteiso-C17) are typically the most abundant and anteiso-C15 fatty acid is the single most abundant fatty acid in B. subtilis. The odd-numbered iso-fatty acids (iso-C15 and iso-C17) are next in order of abundance, and the even-numbered iso- (iso-C14 and iso-C16) and straight-chain (n-C14 and n-C16) fatty acids are of relatively low and variable abundance, respectively.

In E. coli, the majority of the fatty acids (FA) produced are straight-chain and range from C14-C18 in carbon length (Sullivan, 1979, J. Bac, Alteration of FA composition of E. coli by growth in presence of alcohols, V138(1), p. 133-138; Shaw, 1965, J. Bac., Fatty acid composition of E. coli as a possible control factor of minimal growth temperature, V90(1), p. 141-146). The fatty acids of C16 length (C16:0 at about 30.95-38.6% and C16:1 at about 27.9-31.45%) are the most abundant pair of acids in E. coli. Unsaturated, C18 fatty acid (C18:1 at about 19.5-27.1%) is next in order of abundance while C14 and C17 fatty acids were of relatively low abundance at about 5.1-5.5% and 3-4.9%, respectively.

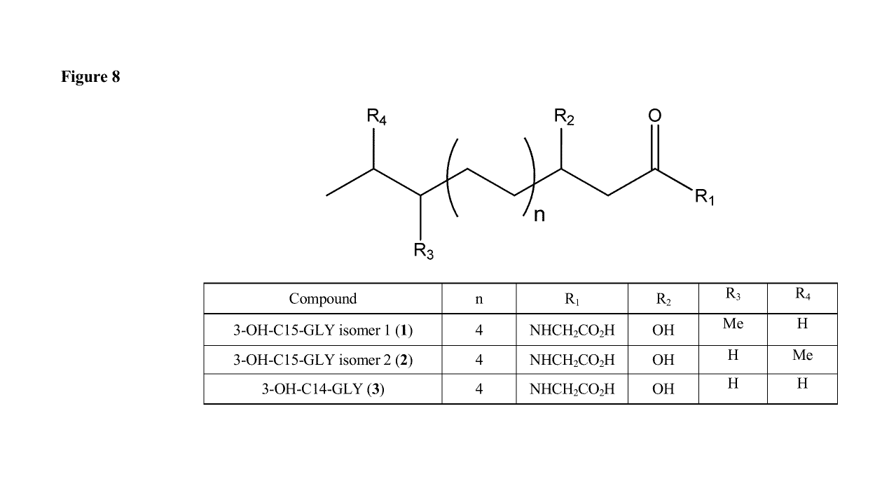

In embodiments the N-acylglycine surfactant produced in a microorganism can be composed of an N-acylglycine surfactant comprising, but not limited to: anteiso-C15-N-acylglycine surfactant; anteiso-C17-N-acylglycine surfactant; iso-C15-N-acylglycine surfactant; iso-C17-N-acylglycine surfactant; iso-C14-N-acylglycine surfactant; iso-C16-N-acylglycine surfactant; straight-chain-C14-N-acylglycine surfactant; straight-chain-C16-N-acylglycine surfactant; straight-chain-C17-N-acylglycine surfactant; C16:1 cis9-N-acylglycine surfactant; C16:1 cis5-N-acylglycine surfactant; unsaturated-C18:1-N-acylglycine surfactant; C16-N-acylglycine surfactant; C16:1-N-acylglycine surfactant; and, iso-C17:1 cis7-N-acylglycine surfactant. In further embodiments, the N-acylglycine surfactant produced in a microorganism can be composed of an N-acylglycine surfactant comprising the 3-OH—C15-GLY isomer 1-N-acylglycine surfactant of FIG. 8 and the 3-OH—C15-GLY isomer 2-N-acylglycine surfactant of FIG. 8. In additional embodiments, the N-acylglycine surfactant produced in a microorganism can be composed of; linear-C13-N-acylglycine surfactant, iso-C13-N-acylglycine surfactant, anteiso-C13-N-acylglycine surfactant, linear-C14-N-acylglycine surfactant, iso-C14-N-acylglycine surfactant, iso-C15-N-acylglycine surfactant, anteiso-C15-N-acylglycine surfactant, and iso-C16-N-acylglycine surfactant. As described above, the pool of fatty acids are known to be produced in the microorganism, and can serve as a pool of fatty acids that can be converted by a chimeric nonribosomal peptide synthetase enzymatic protein of the subject disclosure into an N-acylglycine surfactant, wherein the N-acylglycine surfactant is comprised of a varying chain lengths, branching and addition of chemical moieties. The production of such N-acylglycine surfactant molecules are taught herein as an embodiment of the subject disclosure.

An NRPS protein is a modular multi-domain enzyme that can selectively bind and condense amino acids to enzymatically produce products of varied structural and functional diversity. The number of modules and their organization comprising the NRPS determines the primary structure of the corresponding peptide products. A component of each module of the NRPS machinery is an ‘adenylation domain (A-domain)’ that recognizes a specific amino acid building block and recruits the amino acid to a peptidyl or amino-acyl chain. Disclosed herein are compositions and methods for exchanging the native A-domain of a NRPS protein for a glycine specific A domain. Accordingly, the resulting chimeric NRPS fusion protein was designed to specifically recognize the amino acid glycine. Unexpectedly, the chimeric NRPS fusion protein successfully enabled the in vivo acylation of the amino acid glycine into a medium chain-length β-hydroxy fatty acid peptide chain. As such, when the chimeric NRPS fusion protein was expressed in a prokaryotic species, the bacterial strain was cultured and fermented to result in the production of the non-native lipoamino acid, N-acylglycine biosurfactant. Disclosed herein are embodiments of novel, chimeric NRPS fusion proteins and polynucleotides which encode such proteins.

In an embodiment, the subject disclosure relates to a fusion protein comprising a glycine-specific adenylation domain. By being “glycine specific”, the A-domain of the disclosure only incorporates the amino acid glycine into a medium chain-length β-hydroxy fatty acid peptide chain. The ‘adenylation domain (A-domain)’ recognizes specific amino acid building block that it activates by adenylation and recruits into a growing peptidyl or aminoacyl chain. Exemplary glycine specific adenylation domains are disclosed herein and include the DHBF protein sequence (SEQ ID NO:9) or the dhbF coding sequence (Table 1); the BAEJ protein sequence (SEQ ID NO:90) or the baeJ coding sequence (Table 1); the PKSJ (BSU17180) protein sequence (SEQ ID NO:11) or the pksJ (BSU17180) coding sequence (Table 1); the DptA protein sequence (SEQ ID NO:92) or the dptA coding sequence coding sequence (Table 1); and, the DptBC protein sequence (SEQ ID NO:94) or the dptBC coding sequence (Table 1). Disclosed herein as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:1.0. Further disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:12. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:58. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:59. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:60. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:61. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:62. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:63. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:64. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:65. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID. NO:66. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:91. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:93. Disclosed as an embodiment are glycine specific adenylation domains comprising a protein sequence that shares at least 80%, 82.5%, 87.5%, 90%, 92.5%, 95%, 97.5%, 99%, or 99.5% sequence identity with SEQ ID NO:95. Further disclosed as an embodiment are glycine specific adenylation domains comprising a polynucleotide sequence that shares at least 80%, 82.5%, 87.5%, 90%, 92.5%, 95%, 97.5%, 99%, or 99.5% sequence identity with SEQ ID NO:49. Disclosed as an embodiment are glycine specific adenylation domains comprising a polynucleotide sequence that shares at least 80%, 82.5%, 87.5%, 90%, 92.5%, 95%, 97.5%, 99%, or 99.5% sequence identity with SEQ ID NO:52.

Furthermore, as an embodiment the specific glycine adenylation domain may embody a protein motif. In a subsequent embodiment, the specific glycine adenylation domain is aligned with the gramicidin S synthetase (GrsA) phenylalanine activating domain (Challis G L, Ravel J, Townsend C A. Predictive, structure-based model of amino acid recognition by nonribosomal peptide synthetase adenylation domains. Chem Biol. 2000 March; 7(3):211-24; and, Stachelhaus T, Mootz H D, Marahiel M A. The specificity-conferring code of adenylation domains in nonribosomal peptide synthetases. Chem Biol. 1999 August; 6(8):493-50). In an embodiment, the resulting alignment of the specific glycine adenylation domain with GrsA results in a protein motif of SEQ ID NO:42 wherein: D aligns at amino acid residue 235 of GrsA; I aligns at amino acid residue 236 of GrsA; L aligns at amino acid residue 239 of GrsA; Q aligns at amino acid residue 278 of GrsA; L aligns at amino acid residue 299 of GrsA; G aligns at amino acid residue 301 of GrsA; V aligns at amino acid residue 322 of GrsA; I aligns at amino acid residue 330 of GrsA; W aligns at amino acid residue 331 of GrsA; and, K aligns at amino acid residue 517 of GrsA. The protein motif of SEQ ID NO:42 is included as an embodiment of this disclosure. In an embodiment, the resulting alignment of the specific glycine adenylation domain with GrsA results in a protein motif of SEQ ID NO:41 wherein: D aligns at amino acid residue 235 of GrsA; I aligns at amino acid residue 236 of GrsA; L aligns at amino acid residue 239 of GrsA; Q aligns at amino acid residue 278 of GrsA; L aligns at amino acid residue 299 of GrsA; G aligns at amino acid residue 301 of GrsA; L aligns at amino acid residue 322 of GrsA; I aligns at amino acid residue 330 of GrsA; W aligns at amino acid residue 331 of GrsA; and, K aligns at amino acid residue 517 of GrsA. The protein motif of SEQ ID NO:41 is included as an embodiment of this disclosure. In a further embodiment, the resulting alignment of the specific glycine adenylation domain with GrsA results in a protein motif of SEQ ID NO:40 wherein: D aligns at amino acid residue 235 of GrsA; I aligns at amino acid residue 236 of GrsA; L aligns at amino acid residue 239 of GrsA; Q aligns at amino acid residue 278 of GrsA; L aligns at amino acid residue 299 of GrsA; G aligns at amino acid residue 301 of GrsA; M aligns at amino acid residue 322 of GrsA; I aligns at amino acid residue 330 of GrsA; W aligns at amino acid residue 331 of GrsA; and, K aligns at amino acid residue 517 of GrsA. The protein motif of SEQ ID NO:40 is included as an embodiment of this disclosure. In an additional embodiment, the resulting alignment of the specific glycine adenylation domain with GrsA results in a protein motif of SEQ ID NO:89 wherein: D aligns at amino acid residue 235 of GrsA; I aligns at amino acid residue 236 of GrsA; L aligns at amino acid residue 239 of GrsA; Q aligns at amino acid residue 278 of GrsA; V aligns at amino acid residue 299 of GrsA; G aligns at amino acid residue 301 of GrsA; M aligns at amino acid residue 322 of GrsA; I aligns at amino acid residue 330 of GrsA; W aligns at amino acid residue 331 of GrsA; and, K aligns at amino acid residue 517 of GrsA. The protein motif of SEQ ID NO:89 is included as an embodiment of this disclosure. In an embodiment, the resulting alignment of the specific glycine adenylation domain with GrsA results in a protein motif of SEQ ID NO:1 wherein: D aligns at amino acid residue 235 of GrsA; I aligns at amino acid residue 236 of GrsA; L aligns at amino acid residue 239 of GrsA; Q aligns at amino acid residue 278 of GrsA; L or V align at amino acid residue 299 of GrsA; G aligns at amino acid residue 301 of GrsA; L or M or V align at amino acid residue 322 of GrsA; I aligns at amino acid residue 330 of GrsA; W aligns at amino acid residue 331 of GrsA; and, K aligns at amino acid residue 517 of GrsA. The protein motif of SEQ ID NO:1 is included as an embodiment of this disclosure. Upon completion of an alignment and analysis of the glycine adenylation protein domain sequence with gramicidin S synthetase (GrsA) phenylalanine activating domain, several protein motifs were identified that defined conserved regions that are designated as consensus sequences. These five consensus sequences (SEQ ID NO:42, SEQ ID NO:41, SEQ ID NO:40, SEQ ID NO:1 and SEQ ID NO:89) were determined to define motifs that are characteristic of glycine adenylation protein. Using these motifs to search databases (e.g. GeneBank), one practiced in the art may identify additional putative glycine adenylation genes or proteins from a variety of different organisms.

In further embodiments, the glycine adenylation domain is operably linked to a second domain. For example, the glycine adenylation domain may be operably linked to another NRPS domain. Exemplary NRPS domains may include a condensation domain, a peptidyl carrier protein domain, a thioesterase domain, and/or the type II thioesterase domain.

Disclosed herein as an embodiment is a condensation domain comprising a polynucleotide sequence that shares at least 80%, 82.5%, 87.5%, 90%, 92.5%, 95%, 97.5%, 99%, or 99.5% sequence identity with SEQ ID NO:50. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:67. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:68. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:69. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:70. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:71. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:72. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:73. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:74. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:75. Disclosed herein as an embodiment is a condensation domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:76.

Disclosed herein as an embodiment is a peptidyl carrier protein domain comprising a polynucleotide sequence that shares at least 80%, 82.5%, 87.5%, 90%, 92.5%, 95%, 97.5%, 99%, or 99.5% sequence identity with SEQ ID NO:51. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:77. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:78. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:79. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:80. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:81. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:82. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:83. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:84. Disclosed herein as an embodiment is a peptidyl Carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:85. Disclosed herein as an embodiment is a peptidyl carrier protein domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:86.

Disclosed herein as an embodiment is a thioesterase domain comprising a polynucleotide sequence that shares at least 80%, 82.5%, 87.5%, 90%, 92.5%, 95%, 97.5%, 99%, or 99.5% sequence identity with SEQ ID NO:51. Disclosed herein as an embodiment is a thioesterase domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:87. Disclosed herein as an embodiment is a thioesterase domain comprising a polynucleotide encoding a polypeptide sequence that shares at least 80%, 82.5%, 87.5%, 90%, 92.5%, 95%, 97.5%, 99%, or 99.5% sequence identity with SEQ ID NO:88.

The above described exemplary NRPS domains are found as surfactant gene cluster in microorganisms. For instance Bacillus subtilis contains a surfactant gene cluster. In an embodiment, the surfactant gene clusters obtained from Bacillus subtilis may comprise theSrfAA-M3 (SEQ ID NO:4), SrfAA-M2 (SEQ ID NO:3), SrfAA-M1 (SEQ ID NO:2), SrfAB-M6 (SEQ ID NO:7), SrfAB-M5 (SEQ ID NO:6), SrfAB-M4 (SEQ ID NO:5), and SrfAC-M7 (SEQ ID NO:8) gene clusters.

Disclosed herein are chimeric NRPS fusion proteins that contain a glycine adenylation domain fused to a condensation domain, a peptidyl carrier protein domain, a thioesterase domain, and/or the type II thioesterase domain. The introduction of the glycine adenylation domain at a location between the condensation domain and peptidyl carrier protein domain requires that no modifications result in the alteration of secondary or tertiary structure of the protein sequence, such that the fidelity of the chimeric NRPS fusion protein is modified. Such an alteration could result in instability of the protein or decreased expression levels of the protein. Accordingly, an embodiment of the subject disclosure is the identification of a junction sequence located between the adenylation domain and the condensation domain that allows for integration and engineering of the glycine specific adenylation domain downstream of the condensation domain. In an embodiment, the glycine adenylation domain is operably linked to the condensation domain at the amino acid sequence of SEQ ID NO:53—SDAEKQM, wherein E comprises the junction between the glycine adenylation domain and the condensation domain. In an embodiment, the glycine adenylation domain is operably linked to the condensation domain at the amino acid sequence of SEQ ID NO:54—TLISDAEK, wherein E comprises the junction between the glycine adenylation domain and the condensation domain. In yet another embodiment of the subject disclosure is the identification of a junction sequence located between the adenylation domain and the peptidyl carrier protein domain that allows for integration and engineering of the glycine specific adenylation domain upstream of the peptidyl carrier protein domain. In an embodiment, the glycine adenylation domain is operably linked to the peptidyl carrier protein domain at the amino acid sequence of SEQ ID NO:55—IEWDDDFFAL, wherein the third D comprises the junction between the glycine adenylation domain and the peptidyl carrier protein domain. In an embodiment, the glycine adenylation domain is operably linked to the peptidyl carrier protein domain at the amino acid sequence of SEQ ID NO:56—RVGIDDDFFALG, wherein the third D comprises the junction between the glycine adenylation domain and the peptidyl carrier protein domain. In an embodiment, the glycine adenylation domain is operably linked to the peptidyl carrier protein domain at the amino acid sequence of SEQ ID NO:57—WQEVLNVEKAGIF, wherein the N comprises the junction between the glycine adenylation domain and the peptidyl carrier protein domain.

In a further embodiment of the subject disclosure the chimeric fusion NRPS protein is encoded on a polynucleotide of BSU3a (SEQ ID NO:13), BSU4a (SEQ ID NO:14), and dhbF3a (SEQ ID NO:15). In a subsequent embodiment, the chimeric fusion NRPS polynucleotide shares at least 85%, 87.5%, 90%, 92.5%, 95%, 97.5%, 99% or 99.5 with SEQ ID NO:13. In a subsequent embodiment, the chimeric fusion NRPS polynucleotide shares at least 85%, 87.5%, 90%, 92.5%, 95%, 97.5%, 99% or 99.5 with SEQ ID NO:14. In a subsequent embodiment, the chimeric fusion NRPS polynucleotide shares at least 85%, 87.5%, 90%, 92.5%, 95%, 97.5%, 99% or 99.5 with SEQ ID NO:15.

The genes that encode peptide synthetase complexes have a modular structure that parallels the functional domain structure of the complexes (see, for example, Cosmina et al., Mol. Microbiol. 8:821, 1993; Kratzxchmar et al., J. Bacteriol. 171:5422, 1989; Weckermann et al., Nuc. Acids res. 16:11841, 1988; Smith et al., EMBO J. 9:741, 1990; Smith et al., EMBO J. 9:2743, 1990; MacCabe et al., J. Biol. Chem. 266:12646, 1991; Coque et al., Mol. Microbiol. 5:1125, 1991; Diez et al., J. Biol. Chem. 265:16358, 1990).

Hundreds of peptides are known to be produced by peptide synthetase complexes. Such nonribosomally-produced peptides often have non-linear structures, including cyclic structures exemplified by the peptides surfactant (i.e., surfactin), cyclosporin, tyrocidin, and mycobacillin, or branched cyclic structures exemplified by the peptides polymyxin and bacitracin. Moreover, such nonribosomally-produced peptides may contain amino acids not usually present in ribosomally-produced polypeptides such as for example norleucine, beta-alanine and/or ornithine, as well as D-amino acids. Additionally or alternatively, such nonribosomally-produced peptides may comprise one or more non-peptide moieties that are covalently linked to the peptide. As one non-limiting example, surfactant (i.e., surfactin) is a cyclic lipopeptide that comprises a beta-hydroxy fatty acid covalently linked to the first glutamate of the lipopeptide. Other non-peptide moieties that are covalently linked to peptides produced by peptide synthetase complexes are known to those skilled in the art, including for example sugars, chlorine or other halogen groups, N-methyl and N-formyl groups, glycosyl groups, acetyl groups, etc.

In an embodiment of the subject disclosure, expression of the chimeric NRPS fusion protein is driven by a bacterial promoter. Exemplary promoters are known to those with ordinary skill in the art, and may include a pTAC promoter, a LAC promoter, TAC II promoter, or a PsrfA promoter amongst other commonly known bacterial promoters. Exemplary promoters may be constitutive or inducible. In an embodiment the chimeric NRPS fusion protein is operably linked to a bacterial promoter.

In further embodiments, a chimeric NRPS fusion protein operably linked to a bacterial promoter may be cloned into a vector that can then be transformed into the bacterial host cell. Other regulatory elements may be included in a vector (also termed “expression construct”). Such elements include, but are not limited to, for example, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” polypeptide coding sequences, which facilitates identification, separation, purification, and/or isolation of an expressed.

A polypeptide encoding gene according to the present disclosure can include, in addition to the protein coding sequence, the following regulatory elements operably linked thereto: a promoter, a ribosome binding site (RBS), a transcription terminator, translational start and stop signals. Useful RBSs can be obtained from any of the species useful as host cells in expression systems according to the present disclosure, preferably from the selected host cell. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12): 1123-30 (December 2001). In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989)(native RBS sequence of AAGGAAG). Further examples of methods, vectors, and translation and transcription elements, and other elements useful in the present disclosure are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to protein Wilcox

Vectors are known in the art for expressing recombinant proteins in host cells, and any of these may be used for expressing the genes according to the present disclosure. The plasmid vectors may autonomously replicate within the bacterial strain with or without the use of an antibiotic selection agent. Such vectors include, e.g., plasmids, cosmids, and phage expression vectors. Examples of useful plasmid vectors include, but are not limited to, the expression plasmids pBBR1MCS, pDSK519, pKT240, pML122, pPS10, RK2, RK6, pR01600, and RSF1010. Further examples can include pALTER-Ex1, pALTER-Ex2, pBAD/His, pBAD/Myc-His, pBAD/gIII, pCal-n, pCal-n-EK, pCal-c, pCal-Kc, pcDNA 2.1, pDUAL, pET-3a-c, pET 9a-d, pET-1 pET-12a-c, pET-14b, pET15b, pET-16b, pET-17b, pET-19b, pET-20b(+), pET-21a-d(+), pET-22b(+), pET-23a-d(+), pET24a-d(+), pET-25b(+), pET-26b(+), pET-27b(+), pET28a-c(+), pET-29a-c(+), pET-30a-c(+), pET31b(+), pET-32a-c(+), pET-33b(+), pET-34b(+), pET35b(+), pET-36b(+), pET-37b(+), pET-38b(+), pET-39b(+), pET-40b(+), pET411a-c(+), pET-42a-c(+pET43a-c(+), pETBlue-1, pETBlue-2, pETBlue-3, pGEMEX-1, pGEMEX-2, pGEX1λT, pGEX-2T, pGEX-2TK, pGEX-3X, pGEX4T, pGEX-5X, pGEX-6P, pHAT10/11/12, pHAT20, pHAT-GFPuv, pKK223-3, pLEX, pMAL-c2X, pMAL-c2E, pMAL-c2g, pMAL-p2X, pMAL-p2E, pMAL-p2G, pProEX HT, pPROLar.A, pPROTet.E, pQE-9, pQE-16, pQE-30/31/32, pQE40, pQE-50, pQE-70, pQE-80/81/82L, pQE-100, pRSET, and pSE280, pSE380, pSE420, pThioHis, pTrc99A, pTrcHis, pTrcHis2, pTriEx-1, pTriEx-2, pTrxFus. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomblec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001). In addition, Bacillus plasmids, e.g., pDG1662 plasmid, may be obtained from the Bacillus Genetic Stock Center; Biological Sciences 556, 484 W. 12th Ave, Columbus, Ohio 43210-1214.

Transformation of the host cells with the vector(s) disclosed herein may be performed using any transformation methodology known in the art, and the bacterial host cells may be transformed as intact cells or as protoplasts (i.e. including cytoplasts). Exemplary transformation methodologies include ‘poration methodologies, e.g., electroporation, protoplast fusion, bacterial conjugation, and divalent cation treatment (calcium chloride CaCl₂ treatment or CaCl₂/Mg²⁺ treatment), or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). Other known transformation methods specific are described at by Guerout-Fleury, A. M., Frandsen, N. and Stragier, P. (1996) Plasmids for ectopic integration in Bacillus subtilis. Gene 180 (1-2), 57-61.

Embodiments of the disclosure include methods for identifying any neutral site within the bacterial microorganism (i.e., Bacillus subtilis) genome and the integration of a polynucleotide containing a gene expression cassette which is stably expressed.

Other embodiments of the present disclosure can include integrating a polynucleotide into the bacterial microorganism (i.e., Bacillus subtilis) genome without negatively impacting the production, growth or other desired metabolic characteristics of the bacterial microorganism (i.e., Bacillus subtilis).

Other embodiments of the present disclosure can include integrating a polynucleotide into the bacterial microorganism (i.e., Bacillus subtilis) genome at a neutral site, and the subsequent stacking of a second polynucleotide at the same location. Wherein, the neutral site within the bacterial microorganism (i.e., Bacillus subtilis) is utilized as a preferred locus for introducing additional polynucleotides. In an embodiment the amyE genomic locus serves as a neutral integration site for the integration of a polynucleotide into the bacterial microorganism (i.e., Bacillus subtilis) genome.

Other embodiments of the present disclosure can include integrating a polynucleotide containing a gene expression cassette into the bacterial microorganism (i.e., Bacillus subtilis) genome at a neutral site, and the subsequent removal of a selectable marker expression cassette from the integrated polynucleotide. Wherein, the method used to remove the selectable marker expression cassette is a double crossing over method, an excision method using CRE-LOX, an excision method using FLP-FRT, or an excision method using the RED/ET RECOMBINATION® kit (Genebridges, Heidelberg, Germany), in addition to other excision methods known in the art.

Other embodiments of the present disclosure can include integrating a polynucleotide into bacterial microorganism (i.e., Bacillus subtilis) genome at a neutral site as an alternative to the use of extraneous replicating plasmids. Wherein, one or more extraneous replicating plasmids are incompatible due to the presence of similar origins or replication, incompatibility groups, redundant selectable marker, or other gene elements. Wherein, one or more extraneous replicating plasmids are not functional in bacterial microorganism (i.e., Bacillus subtilis) due to the specificity of the bacterial microorganism (i.e., Bacillus subtilis) restriction modification system. Wherein, one or more extraneous replicating plasmids are not available, functional or readily transformable within bacterial microorganism (i.e., Bacillus subtilis).

Other embodiments of the present disclosure can include methods for increasing the efficiency of homologous recombination in a prokaryotic cell. Methods relying upon homologous recombination mediated by introduced enzymes, such as lambda red ‘recombineering’ and analogous approaches are useful in a limited number of bacterial classes, particularly Escherichia (Datsenko and Wanner (2000) Proc Natl Acad Sci USA. 97: 6640-5), Salmonella, and Bacillus. Methods relying upon site-specific recombination mediated by introduced enzymes, such as phage integrases, FLP/FRT or Cre/loxP may also be used, but are reliant on the presence of pre-existing sites within the target DNA (Wirth et al (2007) Current Opinions in Biotechnology 18, 411-419). Alternative methods exploit viruses or mobile elements, or their components (e.g. phage, transposons or mobile introns).

However, methods relying upon host-mediated homologous recombination are by far the most commonly-used type of chromosomal DNA modifications. In a typical microbial application of host-mediated homologous recombination, a plasmid with a single region of sequence identity with the chromosome is integrated into the chromosome by single-crossover integration, sometimes referred to as ‘Campbell-like integration’. After such an event, genes on the introduced plasmid are replicated as part of the chromosome, which may be more rapid than the plasmid replication. Accordingly, growth in medium with selection for a plasmid-borne selectable marker gene may provide a selective pressure for integration. Campbell-like integration can be used to inactivate a chromosomal gene by placing an internal fragment of a gene of interest on the plasmid, so that after integration, the chromosome will not contain a full-length copy of the gene. The chromosome of a Campbell-like integrant cell is not stable, because the integrated plasmid is flanked by the homologous sequences that directed the integration. A further homologous recombination event between these sequences leads to excision of the plasmid, and reversion of the chromosome to wild-type. For this reason, it may be necessary to maintain selection for the plasmid-borne selectable marker gene to maintain the integrant clone.

An improvement on the basic single-crossover integration method of chromosomal modification is double crossover homologous recombination, also referred to as allelic exchange, which involves two recombination events. The desired modified allele is placed on a plasmid flanked by regions of homology to the regions flanking the target allele in the chromosome (‘homology arms’). A first integration event can occur in either pair of homology arms, leading to integration of the plasmid into the chromosome in the same manner as Campbell-like integration. After the first crossover event, the chromosome contains two alternative sets of homologous sequences that can direct a second recombination event. If the same sequences that directed the first event recombine, the plasmid will be excised, and the cell will revert to wild-type. If the second recombination event is directed by the other homology arm, a plasmid will be excised, but the original chromosomal allele will have been exchanged for the modified allele introduced on the plasmid; the desired chromosomal modification will have been achieved. As with Campbell-like integration, the first recombination event is typically detected and integrants isolated using selective advantage conferred by integration of a plasmid-borne selectable marker gene.

As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, a mineral salts media; a rich medium may be used, but is preferably avoided. In another embodiment either a minimal medium or a mineral salts medium is selected. In still another embodiment, a minimal medium is selected. In yet another embodiment, a mineral salts medium is selected. Mineral salts media are particularly preferred. All such media can be utilized for the expression of N-acylglycine surfactants and are considered as a suitable expression medium for microorganism fermentation.

The fermentation system according to the present disclosure can be cultured in any fermentation format. For example, batch, fed-batch, semi-continuous, and continuous fermentation modes may be employed herein.

The fermentation systems according to the present disclosure are useful for transgene expression at any scale (i.e. volume) of fermentation. Thus, e.g., microliter-scale, centiliter scale, and deciliter scale fermentation volumes may be used. In addition, larger scale fermentations including fermentations greater than 1 Liter scale can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 50 Liters, 1,000 Liters, 2,000 Liters, 5,000 Liters, 10,000 Liters, 50,000 Liters or 100,000 Liters.

In the present disclosure, growth, culturing, and/or fermentation of the transformed host cells is performed within a temperature range permitting survival of the host cells, preferably a temperature within the range of about 4° C. to about 55° C., inclusive.

The ability for a microorganism to produce N-acylglycine surfactants according to this disclosure may be further assayed by isolating and purifying chimeric nonribosomal peptide synthetase proteins to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, column chromatography, immunopurification methods, and others. For example, N-acylglycine surfactants having established molecular adhesion properties can be reversibly fused to a ligand. With the appropriate ligand, the N-acylglycine surfactants can be selectively adsorbed to a purification column and then freed from the column in a relatively pure form. The fused N-acylglycine surfactants is then removed by enzymatic activity. In addition, protein can be purified using immunoaffinity columns or Ni-NTA columns. General techniques are further described in, for example, R. Scopes, Protein Purification: Principles and Practice, Springer-Verlag: N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); A K Patra et al., Protein Expr Purif, 18(2): p/ 182-92 (2000); and R. Mukhija, et al., Gene 165(2): p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example, Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QIAGEN, Inc., Chatsworth, Calif.

The recombinantly produced and expressed N-acylglycine surfactants can be recovered and purified from the recombinant cell cultures by numerous methods, for example, high performance liquid chromatography (HPLC) can be employed for final purification steps, as necessary.

The molecular weight of a N-acylglycine surfactant can be used to isolate it from cellular debris of greater or lesser size using ultrafiltration through membranes of different pore size (for example, Amicon or Millipore membranes). As a first step, the N-acylglycine surfactant mixture can be ultrafiltered through a membrane with a pore size that has a lower molecular weight cut-off than the molecular weight of the N-acylglycine surfactant. The retentate of the ultrafiltration can then be ultrafiltered against a membrane with a molecular cut off greater than the molecular weight of the N-acylglycine surfactant. The N-acylglycine surfactants will pass through the membrane into the filtrate.

N-acylglycine surfactants can also be separated from other cellular debris on the basis of its size, net surface charge, hydrophobicity, and affinity for ligands. In addition, the N-acylglycine surfactants can be conjugated to column matrices for isolation. All of these methods are well known in the art. It will be apparent to one of skill that chromatographic techniques can be performed at any scale and using equipment from many different manufacturers (e.g., Pharmacia Biotech).

Upon isolation and purification of N-acylglycine surfactants, the molecules can be used for, but not limited to, personal care.

In the present disclosure, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, topical medicines, and sunscreens.

In the present disclosure, “personal care” is also intended to refer to hair care compositions including, for example, shampoos, leave-on conditioners, styling gels, hairsprays, and mousses. Preferably, the hair care compositions are cosmetically acceptable.

“Personal care” relates to compositions to be topically administered (i.e., not ingested). Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the disclosure may be manufactured by processes well known in the art, for example, by means of conventional mixing, dissolving, granulating, emulsifying, encapsulating, entrapping or lyophilizing processes.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1

### Identification and Characterization of Glycine-Specific Adenylation Domains

Amino acid specific adenylation domains (“A” domains) were identified from microbial genomic DNA submissions provided in Genbank (Benson, D., Karsch-Mizrachi, I., Lipman, D. J., Ostell, J., and Wheeler, D. L. (2005). GenBank. Nucleic Acids Res. 33, D34-D38.doi:10.1093/nar/gki063). The A domains enzymatically recognize specific amino acid building blocks and recruit the amino acid to a peptidyl or amino-acyl chain. Each A domain specifically recognizes and binds a particular amino acid. Analysis of the isolated A domains was completed to determine the M1 and M5 “A” domains that were glycine-specific A domains. Table 1 lists the glycine specific M1 and M5 “A” domains that were identified from the analysis and search of Genbank.

TABLE 1
Glycine-specific adenylation domains from Bacillus subtilis, Bacillus amyloliquefaciens
and Streptomyces roseosporus. Amino acids lining the substrate binding pocket of the
adenylation domain which confer specificity (corresponding to amino acid residues at positions
235, 236, 239, 278, 299, 301, 322, 330, 331, and 517 of the substrate-binding pocket of the
gramicidin S synthetase (GrsA) phenylalanine activating domain; Challis GL, Ravel J, Townsend
CA. Predictive, structure-based model of amino acid recognition by nonribosomal peptide
synthetase adenylation domains. Chem Biol. 2000 Mar; 7(3): 211-24, and Stachelhaus T,
Mootz HD, Marahiel MA. The specificity-conferring code of adenylation domains in nonribosomal
peptide synthetases. Chem Biol. 1999 Aug; 6(8): 493-505) are listed below.

| | | | | | Conserved Binding Site Residue | | | | | | | | | | |
|---|---|---|---|---|---|---|---|---|---|---|---|---|---|---|---|
| | | | | | | Amino acid residue location | | | | | | | | | |
| | | | Amino Acid | | | as compared to GrsA | | | | | | | | | |
| Organism | Strain | Gene | Specificity | Domain | SEQ ID NO: | 235 | 236 | 239 | 278 | 299 | 301 | 322 | 330 | 331 | 517 |
| B. subtilis | Strain | dhbF | glycine | M1 | SEQ ID NO: 41 | D | I | L | Q | L | G | L | I | W | K |
| | 168 | | | | | | | | | | | | | | |
| B. | FZB42 | baeJ | glycine | M1 | SEQ ID NO: 40 | D | I | L | Q | L | G | M | I | W | K |
| amyloliquefaciens. | | | | | | | | | | | | | | | |
| B. subtilis | Strain | pksJ | glycine | M1 | SEQ ID NO: 40 | D | I | L | Q | L | G | M | I | W | K |
| | 168 | (BSU17180) | | | | | | | | | | | | | |
| S. roseosporous | NRRL | dptA | glycine | M5 | SEQ ID NO: 42 | D | I | L | Q | L | G | V | I | W | K |
| | 11379 | | | | | | | | | | | | | | |
| S. roseosporous | NRRL | dptBC | glycine | M5 | SEQ ID NO: 89 | D | I | L | Q | V | G | M | I | W | K |
| | 11379 | | | | | | | | | | | | | | |

| | | | | | | | | | | | |
|---|---|---|---|---|---|---|---|---|---|---|---|
| Results in a Conserved Sequence of SEQ ID NO: 1 | SEQ ID NO: 1 | D | I | L | Q | (L/ | G | (L/M/ | I | W | K |
| | | | | | | V) | | V) | | | |

### Example 2

### Identification and Annotation of the B. subtilis Surfactin Biosynthesis Gene Cluster

Next, the sequences of seven different B. subtilis surfactin (also described as surfactant) biosynthesis gene cluster domains were obtained and sequenced from Genbank. The individual domains of the B. subtilis surfactant biosynthesis gene cluster (C: condensation domain, A: adenylation domain, P: peptidyl carrier protein domain, and TE: thioesterase domain) for each seven B. subtilis surfactin biosynthesis gene cluster domains were identified by screening the protein sequences using the PSK/NRPS prediction tool (Brian O. Bachmann and Jacques Ravel (2009) In silico Prediction of Microbial Secondary Metabolic Pathways from DNA Sequence Data. Methods in Enzymology. 458:181-217). In addition, the dhbF and pksJ glycine-specific adenylation domains, which were identified above, were further analyzed using the PSK/NRPS prediction tool. The PSK/NRPS prediction tool identified the various domains for each of the corresponding sequences that are presented in Table 2.

TABLE 2
The seven Bacillus subtilis surfactin biosynthesis gene cluster domains and the
encoding polynucleotide sequence. The individual sub-domains that make up
each domain are abbreviated as follows; C: condensation domain A: adenylation
domain, P: peptidyl carrier protein domain, and TE: thioesterase domain.

| | | | Individual Sub- | | |
|---|---|---|---|---|---|
| | | | domains within | | |
| | Accession | Domain | the Biosurfactant | A-Domain | |
| Organism | Number | Name | Domains | Specificity | SEQ ID NO: |
| B. subtilis | NC_000964.3 | SrfAA - | C-A-P | Glutamate | SEQ ID NO: 2 |
| | | M1 | | | |
| B. subtilis | NC_000964.3 | SrfAA - | C-A-P | Leucine | SEQ ID NO: 3 |
| | | M2 | | | |
| B. subtilis | NC_000964.3 | SrfAA - | C-A-P-E | D-Leucine | SEQ ID NO: 4 |
| | | M3 | | | |
| B. subtilis | NC_000964.3 | SrfAB - | C-A-P | Valine | SEQ ID NO: 5 |
| | | M4 | | | |
| B. subtilis | NC_000964.3 | SrfAB - | C-A-P | Aspartate | SEQ ID NO: 6 |
| | | M5 | | | |
| B. subtilis | NC_000964.3 | SrfAB - | C-A-P-E | D-Leucine | SEQ ID NO: 7 |
| | | M6 | | | |
| B. subtilis | NC_000964.3 | SrfAC - | C-A-P-TE | Leucine | SEQ ID NO: 8 |
| | | M7 | | | |

### Example 3

### Organization and Arrangement of Chimeric NRPS Coding Sequences

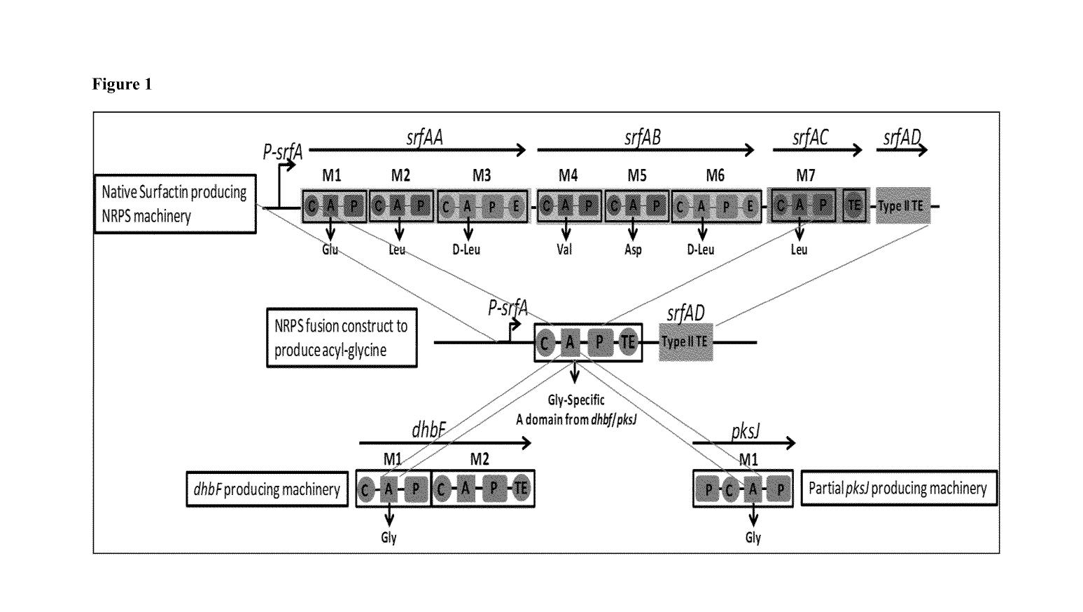

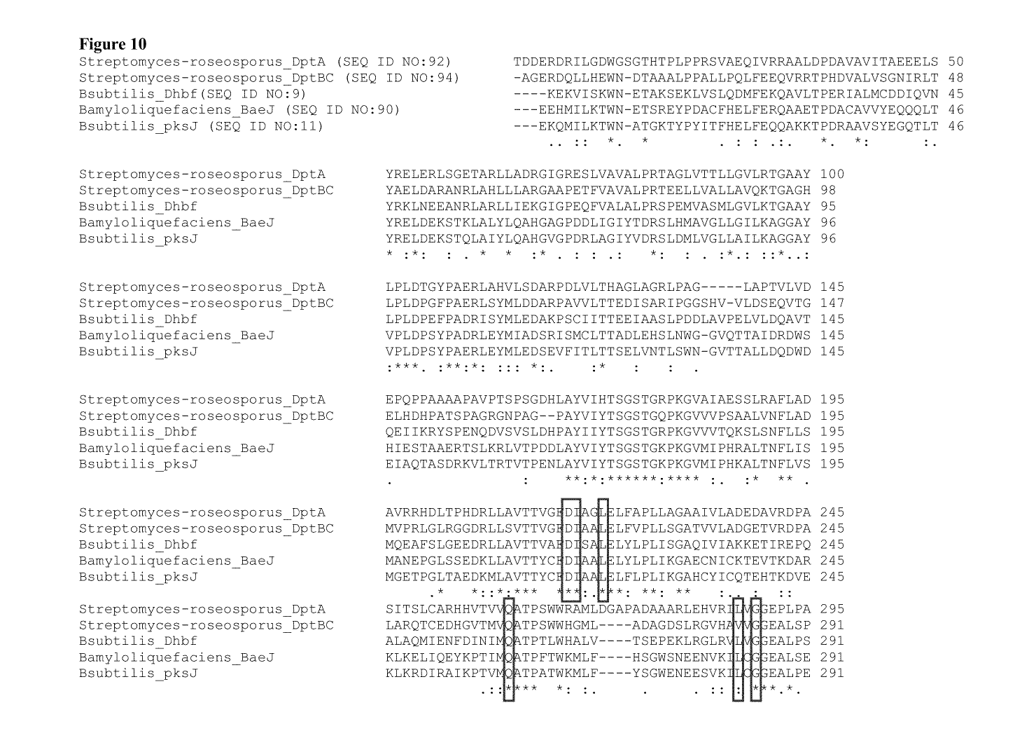

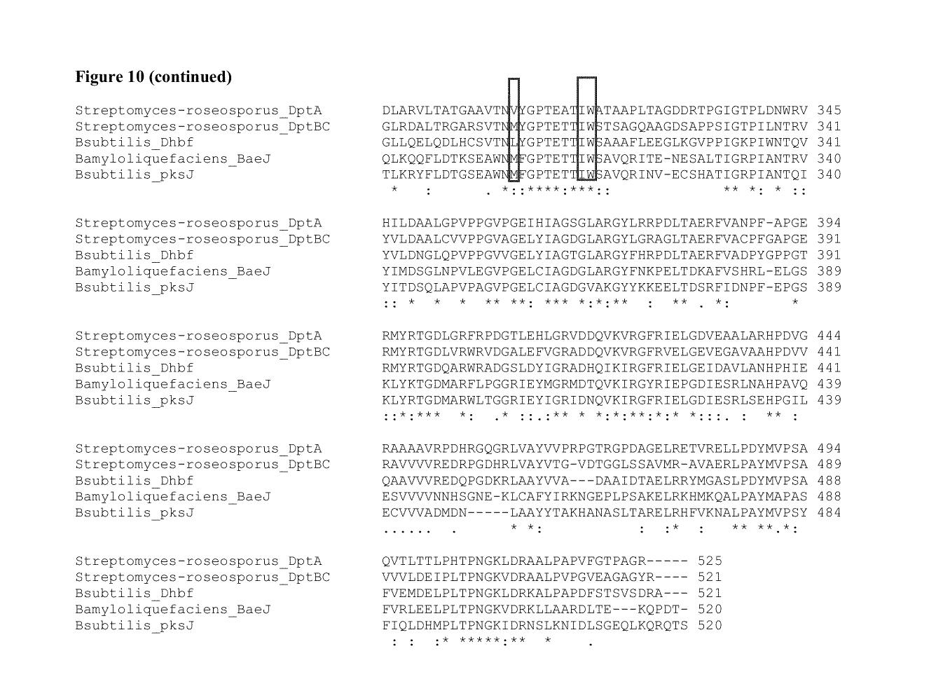

After the glycine-specific adenylation domains and the various domains of the surfactin biosynthesis gene cluster were identified, they were incorporated as a chimeric, fusion gene to produce a novel, chimeric coding sequence. Accordingly, the functional condensation (C) domain, the identified glycine-specific adenylation (A) domain, the peptidyl carrier protein (P) domain, and the thioesterase (TE) domain were designed as a chimeric, single contiguous open reading frame (i.e., C-A-P-TE). In the initial design, the C, P and TE domain coding sequences were obtained from the SrfAA (SEQ ID N0:43), SrfAB (SEQ ID N0:44), and SrfAC (SEQ ID N0:45) modules of the native surfactin biosynthetic cluster of B. subtilis str. OKB120. An alignment of the protein sequences (FIG. 10) from the coding sequences of the A domains recognizing glycine were identified from the following genes: dhbF (B. subtilis; NC_000964.3; gi|254763440|sp|P45745.4|; A domain of SEQ ID N0:9 from protein SEQ ID N0:10); pksJ (B. subtilis 168; NC_000964.3; gi|255767396|ref|NP_389598.3|; A domain of protein SEQ ID NO:11 from protein SEQ ID N0:12); baeJ (B. amyloliquefaciens, gi|154686131|ref|YP_001421292.1|; A domain of SEQ ID N0:90 from protein SEQ ID N0:91); dptA (S. roseosporous; gi|60650932|gb|AAX31557.1|; A domain of SEQ ID N0:92 from protein SEQ ID N0:93); and, dptBC (S. roseosporous; gi|60650933|gb|AAX31558.1|; A domain of SEQ ID N0:94 from protein SEQ ID NO:95). FIG. 1 provides a schematic of the organization and arrangement of the NRPS fusion gene subdomains in relation to the srfAD gene. The resulting novel, chimeric NRFS open reading frame was designed to contain the srfAD gene located downstream. The srfAD gene encodes an external type II thioesterase enzyme and has been previously shown to stimulate the formation of the surfactant biosynthesis in B. subtilis.

### Example 4

### Construction of Chimeric NRPS Coding Sequences

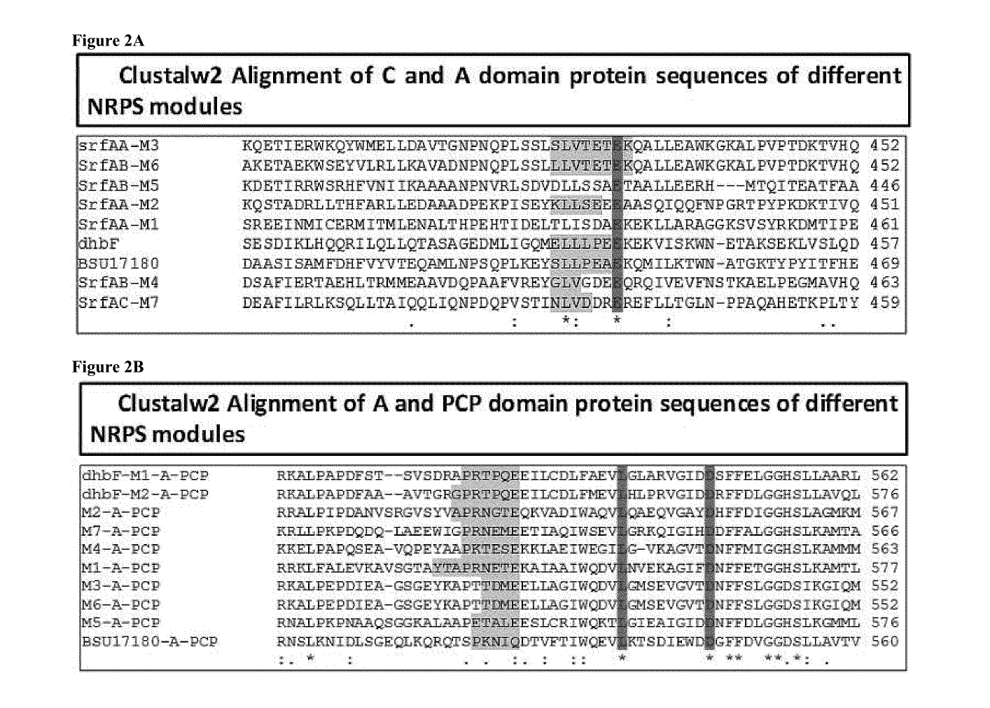

The C, A, and P domain sequences from the biosurfactant genes were identified using the PSK/NRPS prediction tool as described above, and were aligned by the sequence alignment tool, CLUSTALW™. The conserved residues within the different C, A, and P domains as well as at the junctions between domains were identified (FIG. 2). A conserved glutamic-acid (E) residue at the C and A junction was identified and was used to fuse the upstream C domain to the downstream glycine-specific A domain (FIG. 2A; highlighted and labeled with an asterisk). Likewise, a conserved leucine (L) or a conserved aspartic acid (D) residue within the P domain were used to fuse the upstream glycine-specific A domain to the downstream P domain (FIG. 2B; highlighted and labeled with an asterisk).

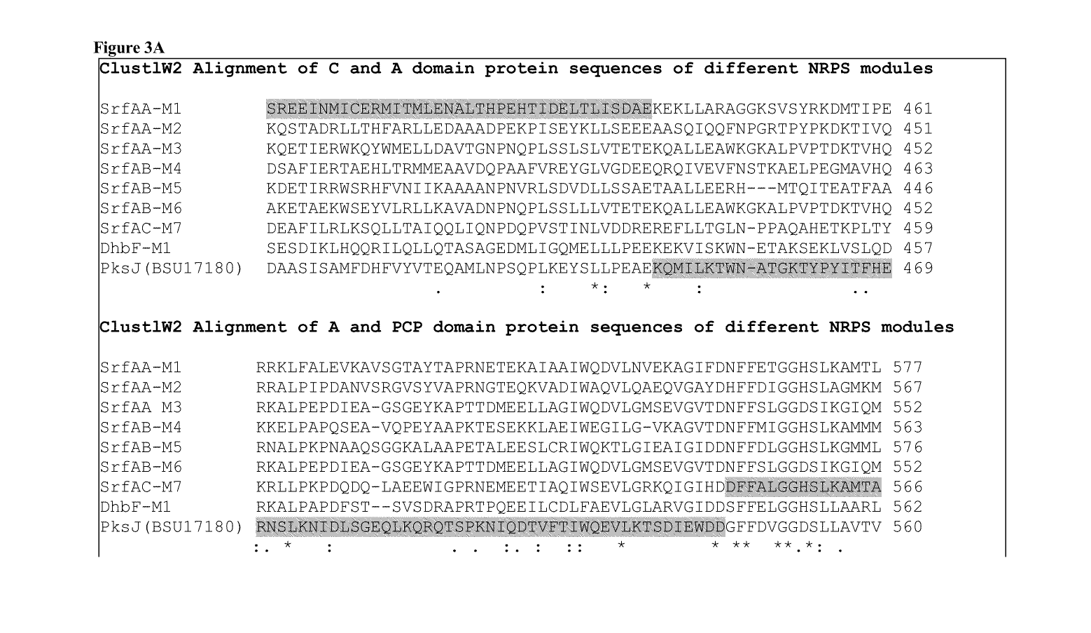

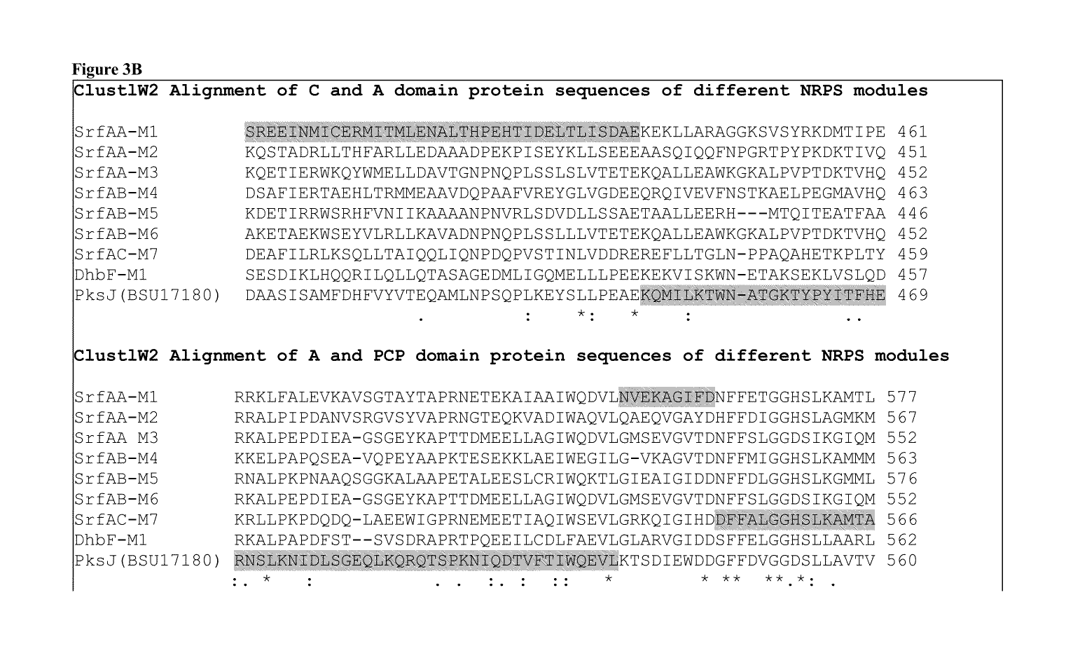

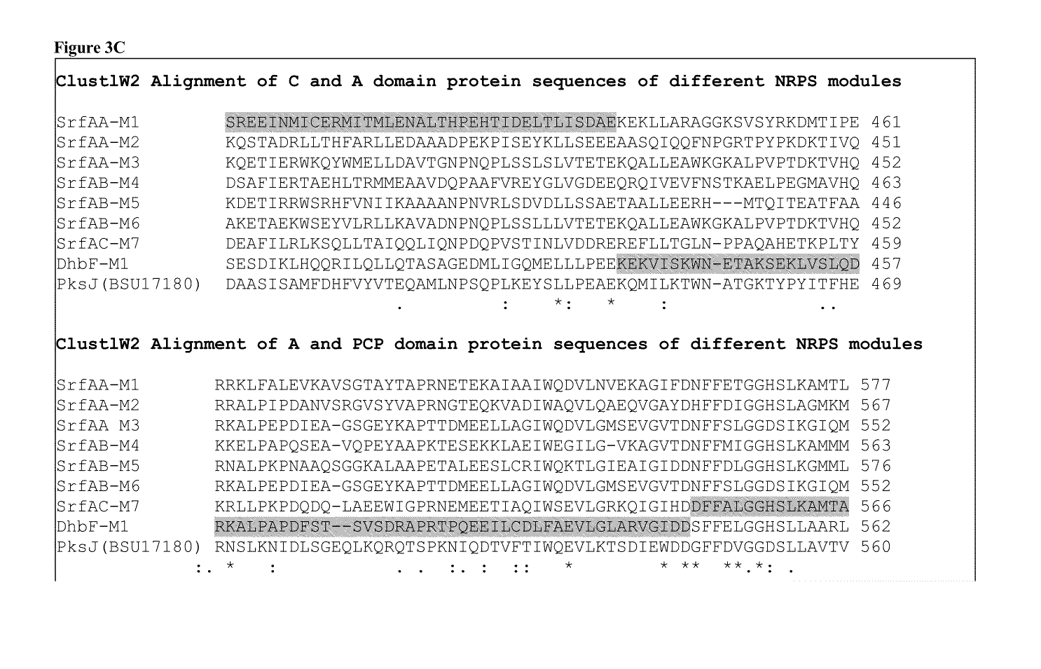

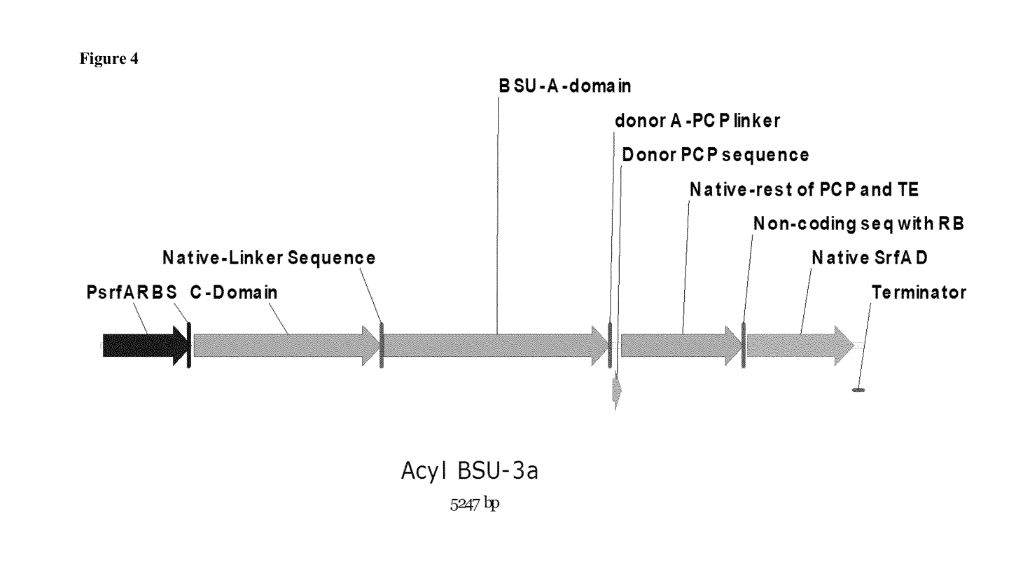

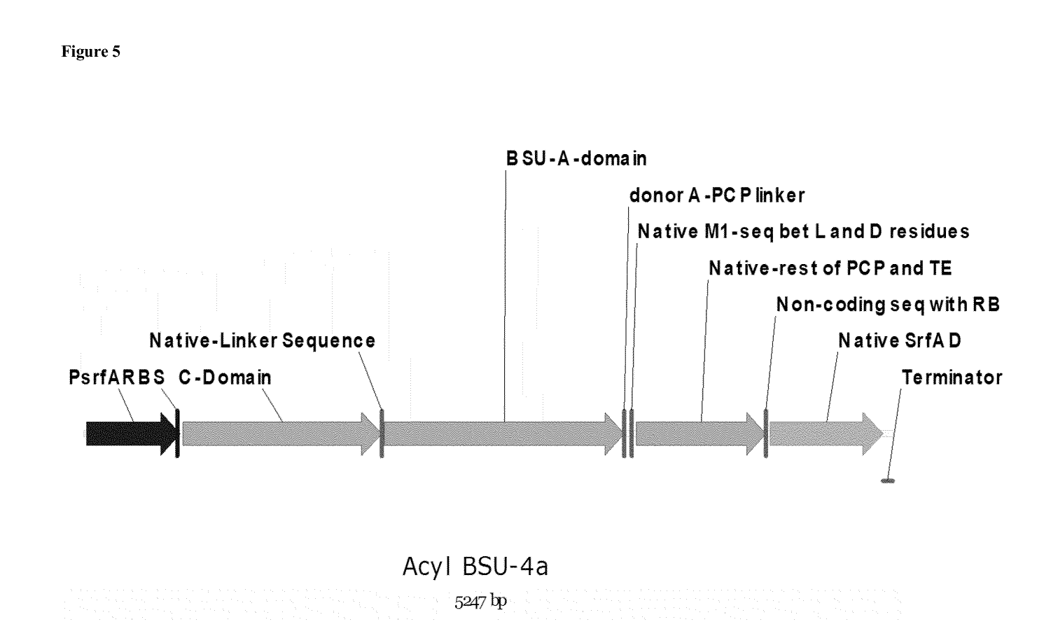

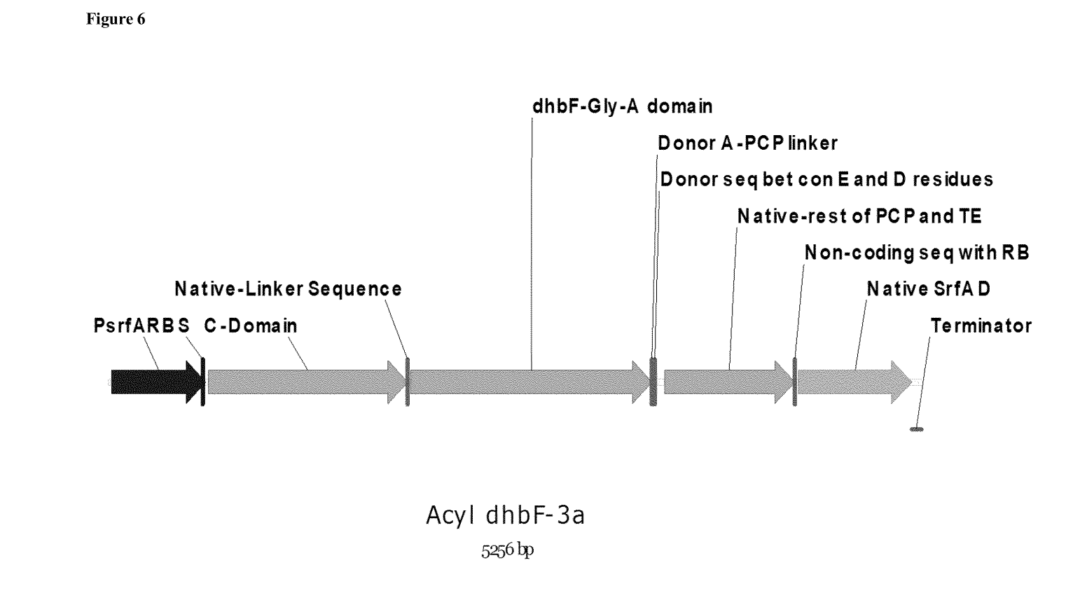

The following three fusion constructs were synthesized using the organization and strategy as previously described in Example 2 (FIG. 3):
(1) BSU3a (SEQ ID NO:13): The glycine-specific A domain sequence originated from pksJ (BSU17180); this A domain (SEQ ID NO:49) was fused downstream of the native surfactin cluster, SrfAA-M1 C domain (SEQ ID NO:50) at the conserved glutamic acid (E) residue within the C and A module junction (FIG. 3A). Next, the A domain of pksJ (BSU17180) was fused to the P domain of SrfAC-M7, such that the P domain was located downstream of the A domain (FIG. 3A). The remaining PCP and TE domain sequences (SEQ ID NO:51) originated from the SrfAC-M1 domain of the native surfactin cluster. The resulting fusion gene construct was cloned downstream of the PsrfA promoter and a ribosome binding stream, and upstream of a native SrfAD coding sequence and bacterial terminator sequence (SEQ ID NO:46). The resulting construct was labeled as ME-B007 (Acyl BSU3a) and is shown as FIG. 4.
(2) BSU4a (SEQ ID NO:14): The glycine-specific A domain originated from pksJ (BSU17180); this A domain (SEQ ID NO:49) was fused downstream of the native surfactin cluster, SrfAA-M1 C domain (SEQ ID NO:50) at the conserved glutamic acid (E) residue within the C and A module junction (FIG. 3B). Next, the A domain of the pksJ (BSU17180) was fused to the chimeric P domain, such that the chimeric P domain was located downstream of the A domain. The chimeric P domain contained the amino acid sequence NVEKAGIFD (SEQ ID NO: 96) from SrfAA-M1 P domain fused to the remaining amino acid sequence of the SrfAC-M7 P domain. The two P domains were joined at a conserved aspartic acid (D) residue (FIG. 3B). The remaining PCP and TE domain sequences (SEQ ID NO:51) originated from the SrfAC-M1 domain of the native surfactin cluster. The resulting fusion gene construct was cloned downstream of the PsrfA promoter and a ribosome binding stream, and upstream of a native SrfAD coding sequence and bacterial terminator sequence (SEQ ID NO:47). The resulting construct was labeled as ME-B008 (Acyl BSU4a) and is shown as FIG. 5.
(3) (SEQ ID NO:15): The glycine-specific A domain originated from the dhbF gene sequence; this A domain (SEQ ID NO:52) was fused downstream of the native surfactin cluster, SrfAA-M1 C domain (SEQ ID NO:50) at the conserved glutamic acid (E) residue within the C and A module junction (FIG. 3C). Next, the A domain of dhbF was fused to the P domain of SrfAC-M7 at a conserved aspartic acid (D) residue, such that the P domain was located downstream of the A domain (FIG. 3C). The remaining P and TE domain sequences (SEQ ID NO:51) came from the SrfAC-M1 domain of the native surfactin gene cluster. The resulting fusion gene construct was cloned downstream of the PsrfA promoter and a ribosome binding stream, and upstream of a native SrfAD coding sequence and bacterial terminator sequence (SEQ ID NO:48). The resulting construct was labeled as ME-B004 (Acyl Dhbf3a) and is shown as FIG. 6.

In addition to the three different fusion gene expression cassettes described above, another 16 chimeric fusion NRPS coding sequences were designed and constructed to assess the effect of different fusion points (i.e., between the C and A domains or the A and P domains) on biosynthesis of the N-acylglycine surfactants as shown in Table 3.

TABLE 3
Chimeric fusion NRPS coding sequences.

| Construct | A domain | | SEQ ID | | |
|---|---|---|---|---|---|
| Name | source | Description | NO: | Promoter | Vector |
| ME-B0014 | dhbf | -------L------L2-------------------- | SEQ ID | PsrfA | pDG1662 |
| | | D--------- | NO: 16 | | |
| ME-B0015 | dhbf | -------L-----------------L1--------- | SEQ ID | PsrfA | pDG1662 |
| | | D--------- | NO: 17 | | |
| ME-B0016 | dhbf | -------L------------E---------------- | SEQ ID | PsrfA | pDG1662 |
| | | D-------- | NO: 18 | | |
| ME-B0017 | dhbf | -------L----------P------------------ | SEQ ID | PsrfA | pDG1662 |
| | | D-------- | NO: 19 | | |
| ME-B0018 | dhbf | -------L----------------------------- | SEQ ID | PsrfA | pDG1662 |
| | | D-------- | NO: 20 | | |
| ME-B0019 | dhbf | ----------E---------------L1-------- | SEQ ID | PsrfA | pDG1662 |
| | | D-------- | NO: 21 | | |
| ME-B0020 | dhbf | ----------E----------E--------------- | SEQ ID | PsrfA | pDG1662 |
| | | D-------- | NO: 22 | | |
| ME-B0021 | dhbf | ----------E-------P------------------ | SEQ ID | PsrfA | pDG1662 |
| | | D-------- | NO: 23 | | |
| ME-B0022 | dhbf | ----------E----L2-------------------- | SEQ ID | PsrfA | pDG1662 |
| | | D--------- | NO: 24 | | |
| ME-B0023 | dhbf | -------L-----------------------G----- | SEQ ID | PsrfA | pDG1662 |
| | | D--------- | NO: 25 | | |
| ME-B0024 | dhbf | ----------E--------------------G----- | SEQ ID | PsrfA | pDG1662 |
| | | D--------- | NO: 26 | | |
| ME-B0025 | pksJ | -------L--------------------L1------- | SEQ ID | PsrfA | pDG1662 |
| | | D--------- | NO: 27 | | |
| ME-B0026 | pksj | -------L------------------W------------ | SEQ ID | PsrfA | pDG1662 |
| | | D--------- | NO: 28 | | |
| ME-B0027 | pksj | -------L---------V------------------- | SEQ ID | PsrfA | pDG1662 |
| | | D--------- | NO: 29 | | |
| ME-B0028 | pksJ | ----------E-----------W------------ | SEQ ID | PsrfA | pDG1662 |
| | | D--------- | NO: 30 | | |
| ME-B0029 | pksJ | ----------E------V------------------- | SEQ ID | PsrfA | pDG1662 |
| | | D--------- | NO: 31 | | |

### Example 5

### Assembly of NRPS Fusion Construct

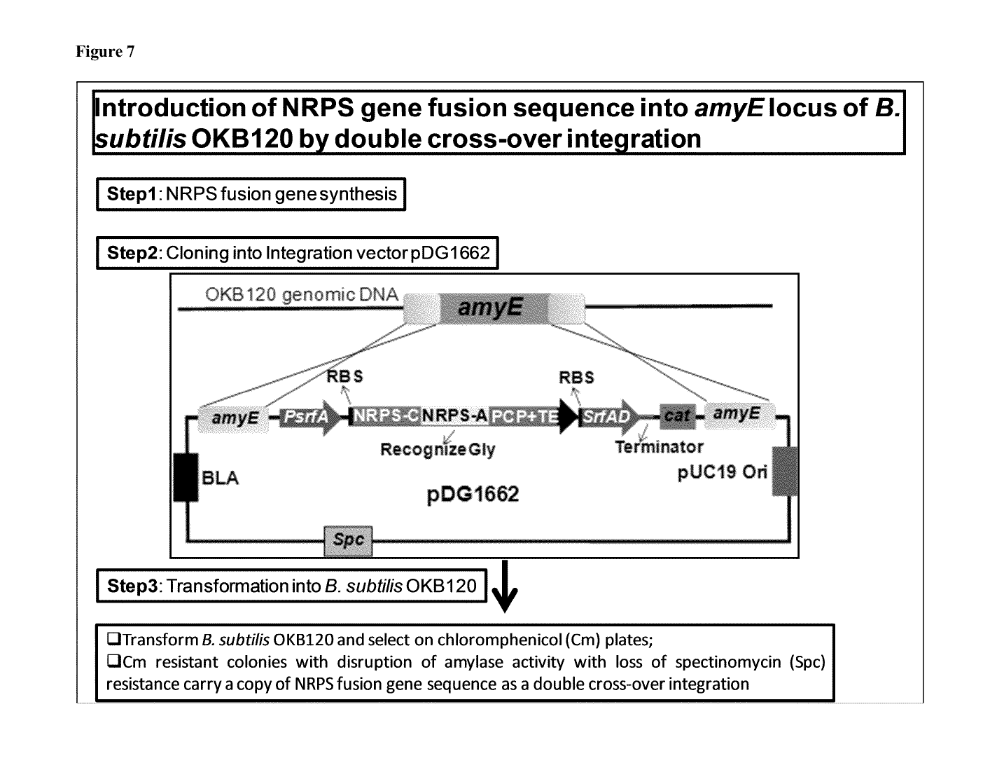

The above described fusion constructs were synthesized and assembled under the expression of the constitutive promoter, PsrfA (SEQ ID NO:32) and flanked by the srdAD coding sequence (SEQ ID NO:33). In addition, the constructs contained native B. subtilis genomic DNA flanking sequences on both ends of the construct. The 5′ end of the gene expression cassette contained the 5′ amyE gene sequence from B. subtilis, and the 3′ end of the gene expression cassette contained the 3′ amyE gene sequence from B. subtilis. The flanking genomic DNA fragments were identical to genomic DNA sequences of the α-amylase gene (amyE) from B. subtilis, and were incorporated into the constructs for integration within the genomic locus. The fusion constructs and flanking genomic DNA were cloned into the pDG1662 plasmid (Bacillus Genetic Stock Center; Biological Sciences 556, 484 W. 12th Ave, Columbus, Ohio 43210-1214). FIG. 7 provides a schematic of the resulting plasmid, and a high level overview of the strategy for introducing the NRPSS gene fusion sequences into the amyE locus of B. subtilis str. OKB120.

### Example 6

### Transformation NRPS Fusion Construct in B. subtilis

The genetic make-up of Bacillus subtilis str. OKB120 is described in detail in Dirk Vollenbroich, Neena Mehta, Peter Zuber, Joachim Vater, and Roza Maria Kamp′(1994). Analysis of Surfactin Synthetase Subunits in srfA Mutants of Bacillus subtilis OKB105. Journal of Bacteriology, Vol. 176, No. 2; p. 395-400. This strain was generated by introducing a transposon mutation in the second module of the surfactin cluster (srfAB) of a surfactant producing strain labeled as OKB105. The resulting mutations to the gDNA of the strain are labeled as B. subtilis str. OKB120 (pheA1 sfp srfA::Tn917). The presence of this transposon insertion mutation renders the strain OKB120 incapable of producing the native surfactant product. However, the strain is capable of producing tetrapeptide and shorter Srf fragments including acyl-glutamate. Accordingly, the strain was transformed with the above described plasmids using the protocol as described in Guerout-Fleury, A. M., Frandsen, N. and Stragier, P. (1996) Plasmids for ectopic integration in Bacillus subtilis. Gene 180 (1-2), 57-61.

### Example 7

### Molecular Confirmation of Genomic DNA Integration of the NRPS Fusion Construct within the B. subtilis Genome

After the separate transformation of each gene construct into the B. subtilis chromosome, molecular confirmation assays were completed to confirm the integration of the NRPS fusion gene sequence into the α-amylase gene (amyE) locus of the genome by homologous recombination. Integration of the NRPS fusion gene construct within the amyE genomic locus resulted in the subsequent disruption of the amyE gene function. Accordingly, colony PCR was employed to detect the successful delivery of the NRPS construct within the bacterial chromosome. Table 4 lists the PCR primers used for colony PCR validation to confirm the presence of NRPS fusion construct and the corresponding gene sequences within the genome of B. subtilis. In addition, the disruption of the amyE locus, was validated by assaying amylase production on starch containing plates (Guerout-Fleury, A. M., Frandsen, N. and Stragier, P. (1996) Plasmids for ectopic integration in Bacillus subtilis, Gene 180 (1-2), 57-61). Furthermore, transformants were screened for the loss of spectinomycin resistance, which indicates that a double crossover event had occurred. B. subtilis str. OKB120 strains containing the NRPS fusion genes were obtained for each of the above described constructs and were fermented to produce N-acylglycine.

TABLE 4
The gene sequence information for NRPS
fusion constructs in this study and the PCR
validation primers used in this sturdy.

| Strain/ | |
|---|---|
| Construct | PCR Validation Primers |
| Dhbf3a | (SEQ ID NO: 34) F-CACGAGCATTTCAAATCTTG |
| | (SEQ ID NO: 35) R-CTGAACAATACGGCCTTG |
| BSU3a | (SEQ ID NO: 36) F-CACGAGCATTTCAAATCTTG |
| | (SEQ ID NO: 37) R-CTGAACAATACGGCCTTG |
| BSU4a | (SEQ ID NO: 38) F-CACGAGCATTTCAAATCTTG |
| | (SEQ ID NO: 39) R-CTGAACAATACGGCCTTG |

### Example 8

### Fermentation of N-Acylglycine

Identified bacterial colonies that contained the genomic integrant of the NRPS fusion gene sequence were isolated and cultured in a defined minimal medium (Media C Recipes for Surfactin production in Bacillus subtilis. Bacterial production of antimicrobial biosurfactants by Bacillus subtilis, Keenan Bence Thesis presented in partial fulfillment of the requirements for the Degree of Master of Science in engineering (chemical engineering) in the Faculty of Engineering at Stellenbosch University, Supervisor Prof. K. G. Clarke December 2011). The fermentation was completed at 37° C. at a volume of 30 ml. The fermentation medium was centrifuged and the cell extracts were prepared at 20, 48 and 72 hours using a 3:1 ratio of methanol to whole broth. The cell extracts were concentrated 2.5× in a Speedvac™ and dissolved in methanol for analysis of the presence of the novel product N-acylglycine by LC/MS.

### Example 9

### Quantitation and Structure Validation of N-Acylglycine Products

Metabolites in extracts prepared as described above were analyzed by two methods. Selected metabolites were quantified by separation using UHPLC followed by quantitation using selected ion monitoring (SIM)-mass spectrometry (MS). Identities of metabolites were validated by separation using UHPLC followed by high resolution MS and MS/MS, as described below. The LC-SIM-MS analysis system comprised the following components: G4220A Infinity 1290 binary pump, G4226A Infinity 1290 autosampler, G4212A Infinity 1290 diode array detector with 10 mm path length flow cell (G4212-60008), G1316C thermostated column compartment (TCC) and G6140A single quadrupole mass spectrometer running under Agilent ChemStation (version B.04.02 SP1 [212]). The system was mass calibrated each day of use using the Agilent CheckTune and/or Autotune routines. Operating parameters were as follows: temperature 350° C., nitrogen drying gas flow: 12 L/min; nebulization pressure: 35 psi; capillary voltage: 3000V, fragmentor voltage: 70V. The LC-accurate MS/MS (QTOF-MS) analysis system comprised the following components: Agilent G4220A Infinity 1290 binary pump, HTC-XT Leap-PAL autosampler, G4212A Infinity 1290 diode array detector with 60 mm path length flow cell (G4212-60007), G1316C column compartment at room temperature (approx. 25° C.) and AB Sciex 5600 quadrupole/time of flight (QTOF-MS) mass spectrometer running under Analyst TF software V 1.6, with data interrogation using Peakview V 1.2. The mass spectrometer was calibrated using a commercial APCI negative calibration solution for the AB Sciex system in the negative ionization mode. Mass measurements on eluted metabolites were made using the QTOF-MS instrument for mass spectra, measured to +/−0.001 Da accuracy, for example m/z 300.001+/−0.001 Da. Operating parameters were as follows: full-scan range 100-1000 Da, MS/MS scan range: 100-1000 Da; accumulation time: full-scan 0.15 sec; MS/MS: 0.10 sec; temperature 450-500° C.; ionspray floating voltage: 4500-5500; declustering potential: 80-100; scan event 1: TOF MS full scan collision energy 5-10 eV; scan events 2-4: product ion IDA collision energy 20-35 eV with a spread of 15 eV. MS/MS spectra were acquired using the following targeted inclusion list, corresponding to [M-H]⁻ for each targeted compound: m/z 300.2, 314.2, 356.2, 372.2 and 386.2. For both methods, metabolites in extracts were separated using an Agilent Eclipse Plus C18 (100×3.0 mm; 1.8 μm particle size) column eluted at 0.425 mL/min with a gradient of water-formic acid (99.9:0.1 v/v; “A”) and acetonitrile-formic acid (99.9:0.1 v/v; “B”). The gradient was as follows: 0-1.33 min: A:B=50:50; 1.33-13.33 min linear gradient to A:B=0:100; 13.33-14.67 min hold at A:B=0:100; 14.67-16.00 min linear gradient to A:B=50:50 and hold to 17.33 min.

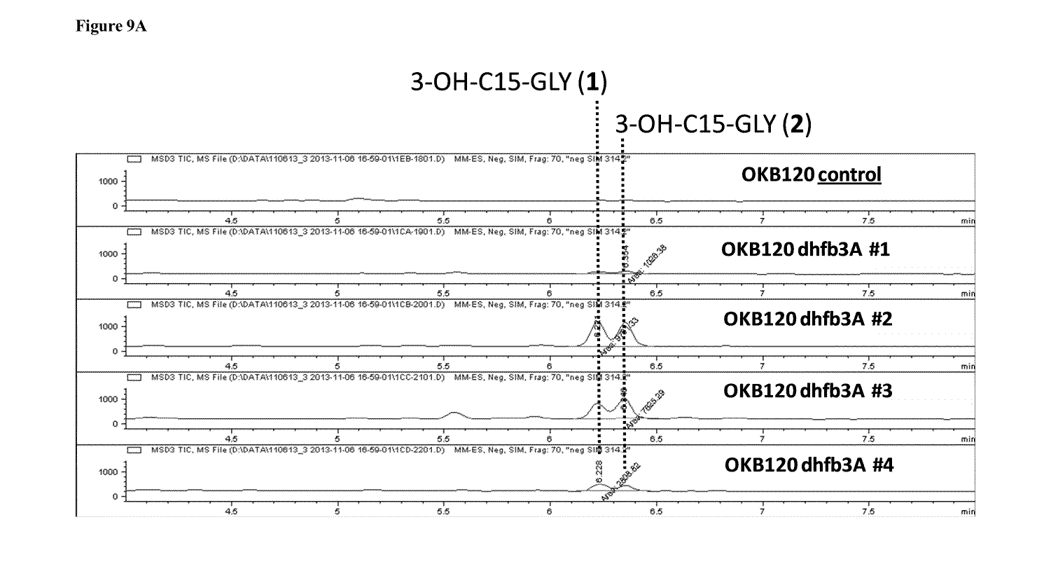

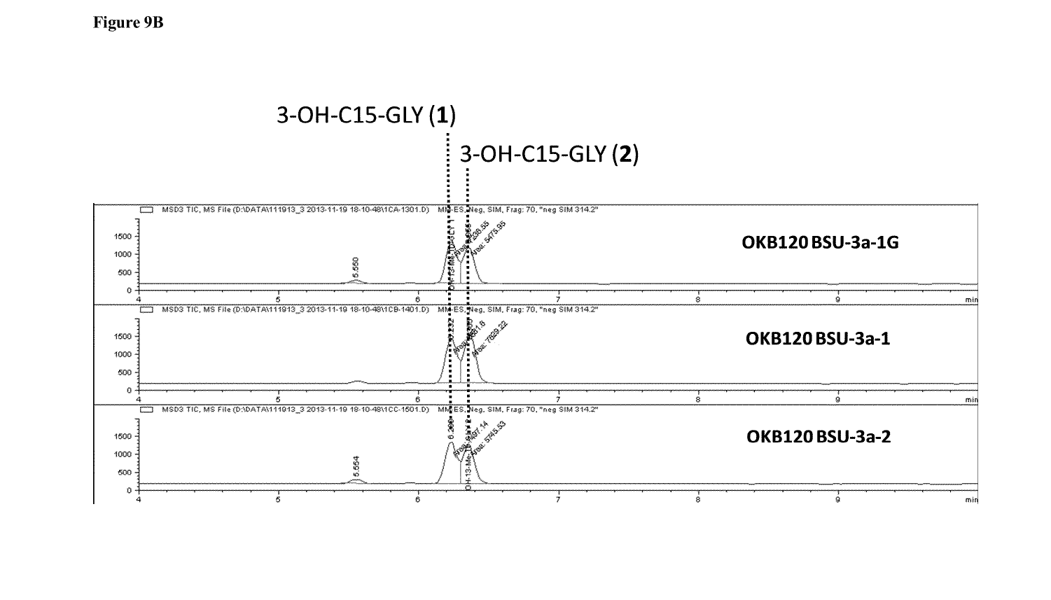

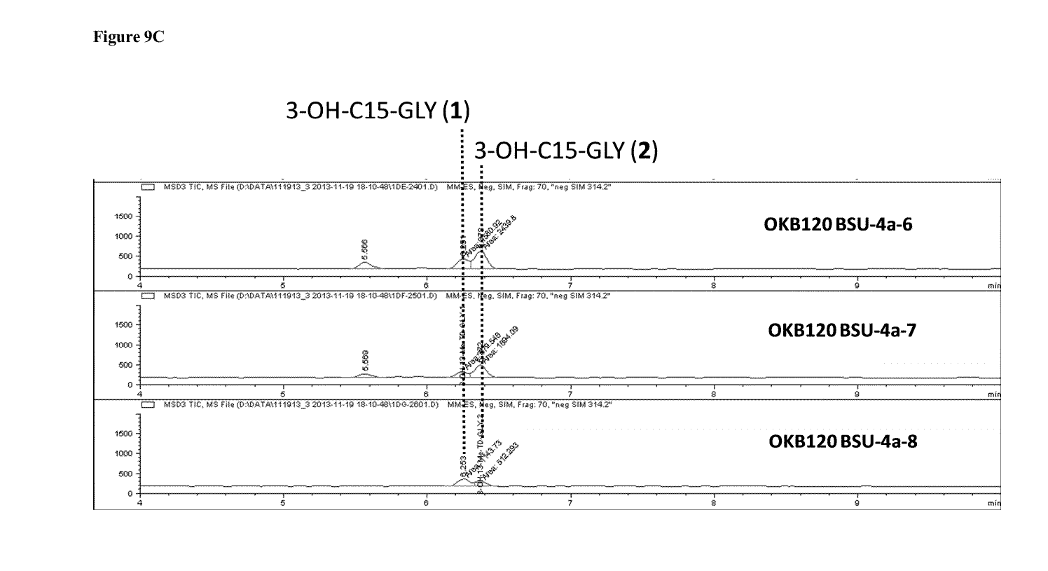

The novel products (1) and (2), shown in FIG. 8, were detected by acquiring a selected ion chromatogram at m/z 314.2, and quantitation was performed with a multi-level calibration curve in external standard mode using authentic 3-OH—C14-GLY (3) (range: 0.001 to 10.136 μg/mL; Matreya LLC Lipids and Biochemicals, Pleasant Gap; PA 16823), which was detected by acquiring a selected ion chromatogram at m/z 300.2. Example chromatograms from the application of this method to extracts of the engineered strains, are shown in FIG. 9A (strain OKB120dhfb3), 9B (OKB120-BSU3a) and 9C (OKB120-BSU4a), which demonstrate successful production of these novel compounds in each of the three constructs. Two chromatographic peaks with the same accurate mass and MS fragments (see below) were observed in the B. subtilis strains during the SIM-MS assay. These peaks were concluded to be isomers of 3-OH—C15-GLY, most likely methyl group positional isomers in the fatty acid chain. In comparison, these two product peaks were not detected in the control (non-engineered) B. subtilis str. OKB120 strains. These data gave a quantitative estimate for combined production levels of 1+2 in the range 96-116 μg/L broth.

A summary of the UHPLC and mass spectral data supporting the structures of the compounds in FIG. 8 produced by strains OKB120-dhbf3a-2 and BSU-3a appears in Table 5. While no authentic standards of the methyl-group isomers 3-OH—C15-GLY (1) or (2) were available for comparison, detection of two compounds having the anticipated EM-Hf ion at m/z 314.234, which eluted closely following an authentic standard of 3-OH—C14-GLY (3), supports the production of the target molecule since an additional methyl group would increase the lipophilicity of the molecule relative to 3-OH—C14-GLY (3), thereby causing it to adsorb slightly more strongly to the UHPLC analysis stationary phase, and elute slightly later. The measured and theoretical weights for the parent ion and all major fragment ions showed good agreement, validating the proposed structures.

TABLE 5
Summary of UHPLC and mass spectral data supporting the structures of the compounds
in FIG. 8 produced by B. subtilis strains OKB120-dhbf3a-2 and BSU-3a.

| | | Retention | Retention | | Proposed | | Measured | Measured |
|---|---|---|---|---|---|---|---|---|
| | | (Mins; | (Mins; | | Molecular | Theoretical | Mass | Mass |
| Sample Type | Compound | Extract) | Standard) | Proposed Ion¹ | Formula | Mass | (Extract) | (Standard) |
| Authentic | 3-OH—C14-GLY (3) | | 5.46 | parent | C₁₆H₃₀NO₄⁻ | 300.218 | | 300.217 |
| Standard | | | | | | | | |
| | | | | M—H₂O | C₁₆H₂₈NO₃⁻ | 282.208 | | 282.207 |
| | | | | M—H₂O—CO₂ | C₁₅H₂₈NO⁻ | 238.218 | | 238.216 |
| | | | | M—C₁₂H₂₄O | C₄H₆NO₃⁻ | 116.035 | | 116.035 |
| OKB120 dhbf3a-2 | 3-OH—C15-GLY | 6.37 | | parent | C₁₇H₃₂NO₄⁻ | 314.234 | 314.237 | |
| extract | isomer 1 (1) | | | | | | | |
| | | | | M—H₂O | C₁₇H₃₀NO₃⁻ | 296.223 | 296.222 | |
| | | | | M—CO₂—H₂ | C₁₆H₃₀NO₂⁻ | 268.228 | 268.236 | |
| | | | | M—H₂O—CO₂ | C₁₆H₃₀NO⁻ | 252.233 | 252.230 | |
| | | | | M—C₁₃H₂₆O | C₄H₆NO₃⁻ | 116.035 | 116.036 | |
| | 3-OH—C15-GLY | 6.52 | | parent | C₁₇H₃₂NO₄⁻ | 314.234 | 314.234 | |
| | isomer 2 (2) | | | | | | | |
| | | | | M—H₂O | C₁₇H₃₀NO₃⁻ | 296.223 | 296.226 | |
| | | | | M—H₂O—CO₂ | C₁₆H₃₀NO⁻ | 252.233 | 252.239 | |
| | | | | M—CH₃CONHCH₂CO₂ | C₁₃H₂₅O⁻ | 197.191 | 197.188 | |
| | | | | H | | | | |
| | | | | M—C₁₃H₂₆O | C₄H₆NO₃⁻ | 116.035 | 116.035 | |
| BSU-3a² extract | 3-OH—C15-GLY | 6.34 | | parent | C₁₇H₃₂NO₄⁻ | 314.234 | 314.236 | |
| | isomer 1 (1) | | | | | | | |
| | | | | M—H₂O | C₁₇H₃₀NO₃⁻ | 296.223 | 296.221 | |
| | | | | M—H₂O—CO₂ | C₁₆H₃₀NO⁻ | 252.233 | 252.228 | |
| | | | | M—C₈H₁₅NO₃ | C₉H₁₇O⁻ | 141.129 | 141.132 | |
| | | | | M—C₁₂H₂₅—OH | C₅H₆NO₃⁻ | 128.035 | 128.041 | |
| | | | | M—C₁₃H₂₆O | C₄H₆NO₃⁻ | 116.035 | 116.037 | |
| | 3-OH—C15-GLY | 6.47 | | parent | C₁₇H₃₂NO₄⁻ | 314.234 | 314.235 | |
| | isomer 2 (2) | | | | | | | |
| | | | | M—H₂O | C₁₇H₃₀NO₃⁻ | 296.223 | 296.224 | |
| | | | | M—H₂O—CO₂ | C₁₆H₃₀NO⁻ | 252.233 | 252.241 | |
| | | | | M—H₂—H₂O—CO₂ | C₁₆H₂₈NO⁻ | 250.218 | 250.214 | |
| | | | | M—C₈H₁₅NO₃ | C₉H₁₇O⁻ | 141.129 | 141.125 | |
| | | | | M—C₁₃H₂₆O | C₄H₆NO₃⁻ | 116.035 | 116.035 | |

¹All parent ions represent [M—H]⁻
²Supplemented with exogenous glycine

In conclusion, LC/MS results demonstrate that B. subtilis str. OKB120 strains expressing the novel, chimeric NRPS fusion proteins can successfully recruit glycine into a medium chain-length 11-hydroxy fatty acid peptide chain, in vivo, resulting in the desired production of N-acylglycine.

### Example 10

### Analysis of Fatty Acid Branching Composition of N-Acylglycine Products

A methanolysis procedure based on the method of Yakimov et al. was used to analyze fatty acids from purified N-acylglycine surfactant products. Briefly, five milligrams of N-acylglycine surfactant product was hydrolyzed in argon purged tubes for 16 h at 90° C. with 20 ml of 25% 12 N HCl in methanol. The hydrolyzed fatty acid methyl esters (FAME) were then extracted with 35 ml of 1:1 (vol/vol) ethyl acetate:hexane. The organic phase was concentrated under a stream of ambient nitrogen to 2 ml. The organic concentrate was then neutralized with 2 ml of 0.4 M phosphate buffer (pH 12) and left to stand until a clear phase separation developed. The upper layer was then transferred into a 2 ml micro-volumetric flask and adjusted to mark as needed. A 0.4 ml aliquot was evaporated under a gentle stream of ambient nitrogen until the point of dryness and derivatized with 0.2 ml neat MSTFA [N-Methyl-N-(trimethylsilyl) trifluoroacetamide] for analysis by gas chromatography-mass spectrometry (Agilent 6890 GC and Agilent 5975C MSD). Next, 1 μl was used for injection and inlet temperature was set at 220° C. in splitless mode. The oven temperature was set at 60° C. for 1 minute, ramped at 4° C./minute to 120° C., 1.5° C./minute to 210° C. with a 4 minute hold, and then ramped at 20° C./minute to 230° C. with a 29 minute hold. The capillary column used was a Thermo Scientific TR-FAME 0.25 mm×120 m×0.25 μm. The carrier gas was helium and the flow rate was 1 ml/minute. The mass spectrometer ion source used methane gas chemical ionization to minimize fragmentation of trimethylsilyl (TMS) derivatized hydroxyl groups on respective FAMEs enhancing intact molecule quantitation and simplifying the spectrum. Retention times, mass spectra, and quantitation were established using authentic fatty acid standards (Matreya LLC, Pleasant Gap, USA) and bacterial reference FAME standards (Sigma-Aldrich, St. Louis, USA). Table 6 provides the results of the N-acylglycine surfactant products and describes the quantitated percentage of branching for each of the fatty acids

TABLE 6
B. subtilis surfactant fatty acid composition and the quantitated
percentage for each of the branched chain fatty acids.

| | Hydroxy fatty acid relative % | | |
|---|---|---|---|
| Carbon | branching composition | | |
| chain | linear | iso | anteiso |

| | | | |
|---|---|---|---|
| 10 | 0.0 | 0.0 | 0.0 |
| 11 | 0.0 | 0.0 | 0.0 |
| 12 | 1.8 | 0.0 | 0.0 |
| 13 | 1.5 | 8.6 | 5.5 |
| 14 | 11.9 | 30.7 | 0.0 |
| 15 | 0.0 | 19.0 | 18.1 |
| 16 | 0.0 | 2.9 | 0.0 |
| 17 | 0.0 | 0.0 | 0.0 |
| 18 | 0.0 | 0.0 | 0.0 |

While aspects of this invention have been described in certain embodiments, they can be further modified within the spirit and scope of this disclosure. This application is therefore intended to cover any variations, uses, or adaptations of embodiments of the invention using its general principles. Further, this application is intended to cover such departures from the present disclosure as come within known or customary practice in the art to which these embodiments pertain and which fall within the limits of the appended claims.

SEQUENCE LISTING

SEQ ID NO: 1

DILQ(L/V)G(L/M/V)IWK
>SrfAA-M1

SEQ ID NO: 2

MEITFYPLTDAQKRIWYTEKFYPHTSISNLAGIGKLVSADAIDYVLVEQAIQEFIRRNDAMR
LRLRLDENGEPVQYISEYRPVDIKHTDTTEDPNAIEFISQWSREETKKPLPLYDCDLFRFSLF
TIKENEVWFYANVHHVISDGISMNILGNAIMHIYLELASGSETKEGISHSFIDHVLSEQEYA
QSKRFEKDKAFWNKQFESVPELVSLKRNASAGGSLDAERFSKDVPEALHQQILSFCEANK
VSVLSVFQSLLAAYLYRVSGQNDVVTGTFMGNRTNAKEKQMLGMFVSTVPLRTNIDGGQ
AFSEFVKDRMKDLMKTLRHQKYPYNLLINDLRETKSSLTKLFTVSLEYQVMQWQKEEDL
AFLTEPIFSGSGLNDVSIHVKDRWDTGKLTIDFDYRTDLFSREEINMICERMITMLENALTH
PEHTIDELTLISDAEKEKLLARAGGKSVSYRKDMTIPELFQEKAELLSDHPAVVFEDRTLSY
RTLHEQSARIANVLKQKGVGPDSPVAVLIERSERMITAIMGILKAGGAYVPIDPGFPAERIQ
YILEDCGADFILTESKVAAPEADAELIDLDQAIEEGAEESLNADVNARNLAYIIYTSGTTGR
PKGVMIEHRQVHHLVESLQQTIYQSGSQTLRMALLAPFHFDASVKQIFASLLLGQTLYIVP
KKTVTNGAALTAYYRKNSIEATDGTPAHLQMLAAAGDFEGLKLKHMLIGGEGLSSVVAD
KLLKLFKEAGTAPRLTNVYGPTETCVDASVHPVIPENAVQSAYVPIGKALGNNRLYILDQ
KGRLQPEGVAGELYIAGDGVGRGYLHLPELTEEKFLQDPFVPGDRMYRTGDVVRWLPDG
TIEYLGREDDQVKVRGYRIELGEIEAVIQQAPDVAKAVVLARPDEQGNLEVCAYVVQKPG
SEFAPAGLREHAARQLPDYMVPAYFTEVTEIPLTPSGKVDRRKLFALEVKAVSGTAYTAP
RNETEKAIAAIWQDVLNVEKAGIFDNFFETGGHSLKAMTLLTKIHKETGIEIPLQFLFEHPTI
TALAE
>SrfAA-M2

SEQ ID NO: 3

IVSQFEDAGVGYNMPAAAILEGPLDIQKLERAFQGLIRRHESLRTSFVLENSTPRQKIHDSV
DFNIEMIERGGRSDEAIMASFVRTFDLAKAPLFRIGLLGLEENRHMLLFDMHHLISDGVSIG
IMLEELARIYKGEQLPDLRLQYKDYAVWQSRQAAEGYKKDQAYWKEVFAGELPVLQLL
SDYPRPPVQSFEGDRVSIKLDAGVKDRLNRLAEQNGATLYMVMLSAYYTLLSKYTGQDD
IIVGTPSAGRNHSDTEGIIGMFVNTLAIRSEVKQNETFTQLISRVRKRVLDAFSHQDYPFEW
LVEDLNIPRDVSRHPLFDTMFSLQNATEGIPAVGDLSLSVQETNFKIAKFDLTVQARETDE
GIEIDVDYSTKLFKQSTADRLLTHFARLLEDAAADPEKPISEYKLLSEEEAASQIQQFNPGR
TPYPKDKTIVQLFEEQAANTPDHTALQYEGESLTYRELNERANRLARGILSLGAGEGRTA
AVLCERSMDMIVSILAVLKSGSAYVPIDPEHPIQRMQHFFRDSGAKVLLTQRKLKALAEEA
EFKGVIVLADEEESYHADARNLALPLDSAAMANLTYTSGTTGTPKGNIVTHANILRTVKE
TNYLSITEQDTILGLSNYVFDAFMFDMFGSLLNGAKLVLIPKETVLDMARLSRVIERENISI
LMITTALFHLLVDLNPACLSTLRKIMFGGERASVEHVRKALQTVGKGKLLHMYGPSESTV
FATYHPVDELEEHTLSVPIGKPVSNTEVYILDRTGHVQPAGIAGELCVSGEGLVKGYYNRP
ELTEEKFVPHPFTSGERMYKTGDLARWLPNGDIEFIGRIDHQVKIRGQRIELGEIEHQLQTH
DRVQESVVLAVDQGAGDKLLCAYYVGEGDISSQEMREHAAKDLPAYMVPAVFIQMDEL
PLTGNGKIDRRALPIPDANVSRGVSYVAPRNGTEQKVADIWAQVLQAEQVGAYDHFFDIG
GHSLAGMKMLALVHQELGVELSLKDLFQSPTVEGLAQVI
>SrfAA-M3

SEQ ID NO: 4

VSSPQKRMYVLQQLEDAQTSYNMPAVLRLTGELDVERLNSVMQQLMQRHEALRTTFEIK
DGETVQRIWEEAECEIAYFEAPEEETERIVSEFIKPFKIDQLPLFRIGLIKHSDTEHVLLFDMH
HIISDGASVGVLIEELSKLYDGETLEPLRIQYKDYAVWQQQFIQSELYKKQEEHWLKELDG
ELPVLTLPTDYSRPAVQTFEGDRIAFSLEAGKADALRRLAKETDSTLYMVLLASYSAFLSK
ISGQDDIIVGSPVAGRSQADVSRVIGMFVNTLALRTYPKGEKTFADYLNEVKETALSAFDA
QDYPLEDLIGNVQVQRDTSRNPLFDAVFSMQNANIKDLTMKGIQLEPHPFERKTAKFDLT
LTADETDGGLTFVLEYNTALFKQETIERWKQYWMELLDAVTGNPNQPLSSLSLVTETEKQ
ALLEAWKGKALPVPTDKTVHQLFEETAQRHKDRPAVTYNGQSWTYGELNAKANRLARI
LMDCGISPDDRVGVLTKPSLEMSAAVLGVLKAGAAFVPIDPDYPDQRIEYILQDSGAKLLL
KQEGISVPDSYTGDVILLDGSRTILSLPLDENDEENPETAVTAENLAYMIYTSGTTGQPKGV
MVEHHALVNLCFWHHDAFSMTAEDRSAKYAGFGFDASIWEMFPTWTIGAELHVIEEAIR
LDrVRLNDYFETNGVTITFLPTQLAEQFMELENTSLRVLLTGGDKLKJIAVKKPYTLVNNY
GPTENTVVATSAEIHPEEGSLSIGRAIANTRVYILGEGNQVQPEGVAGELCVAGRGLARGY
LNREDETAKRFVADPFVPGERMYRTGDLVKWTGGGIEYIGRIDQQVKVRGYRIELSEIEV
QLAQLSEVQDAAVTAVKDKGGNTAIAAYVTPESADIEALKSALKETLPDYMIPAFWVTLN
ELPVTANGKVDRKALPEPDIEAGSGEYKAPTTDMEELLAGIWQDVLGMSEVGVTDNFFSL
GGDSIKGIQMASRLNQHGWKLEMKDLFQHPTIEELTQYV
>SrfAB-M4

SEQ ID NO: 5

LTPMQEGMLYHAMLDPHSSSYFTQLELGIHGAFDLEIFEKSVNELIRSYDILRTVFVHQQL
QKPRQVVLAERKTKVHYEDISHADENRQKEHIERYKQDVQRQGFNLAKDILFKVAVFRL
AADQLYLVWSNHHIMMDGWSMGVLMKSLFQNYEALRAGRTPANGQGKPYSDYIKWLG
KQDNEEAESYWSERLAGFEQPSVLPGRLPVKKDEYVNKEYSFTWDETLVARIQQTANLH
QVTGPNLFQAVWGIVLSKYNFTDDVIFGTVVSGRPSEINGIETMAGLFINTIPVRVKVERD
AAFADIFTAVQQHAVEAERYDYVPLYEIQKRSALDGNLLNHLVAFENYPLDQELENGSM
EDRLGFSIKVESAFEQTSFDFNLIVYPGKTWVKIKYNGAAFDSAFIERTAEHLTRMMEAA
VDQPAAFVREYGLVGDEEQRQIVEVFNSTKAELPEGMAVHQVFEEQAKRTPASTAVVYE
GTKLTYRELNAAANRLARKLVEHGLQKGETAAIMNDRSVETVVGMLAVLKAGAAYVPL
DPALPGDRLRFMAEDSSVRMVLIGNSYTGQAHQLQVPVLTLDIGFEESEAADNLNLPSAPS
DLAYIMYTSGSTGKPKGVMIEHKSILRLVKNAGYVPVTEEDRMAQTGAVSFDAGTFEVFG
ALLNGAALYPVKKETLLDAKQFAAFLREQSITTMWLTSPLFTSTQLAAKDAGMFGTLRHLII
GGDALVPHIVSKVKQASPSLSLWNGYGPTENTTFSTSFLIDREYGGSIPIGKPIGNSTAYIMD
EQQCLQPIGAPGELCVGGIGVARGYVNLPELTEKQFLEDPFRPGERIYRTGDLARWLPDGN
IEFLGRIDNQACWGFRFFTLGEIETKLNMAEHVTEAAVIIRKNKADENEICAYFTADREVAV
SELRKTLSQSLPDYMVPAHLIQMDSLPLTPNGKINKKELPAPQSEAVQPEYAAPKTESEKK
LAEIWEGILGVKAGVTDNFFMIGGHSLKAMMMTAKIQEHFHKEVPIKVLFEKPTIQELAL
YL
>SrfAB-M5

SEQ ID NO: 6

VSPAQRRMYILNQLGQANTSYNVPAVLLLEGEVDKDRLENAIQQLINRHEILRTSFDMIDG
EVVQTVHKNISFQLEAAKGREEDAEEIIKAFVQPFELNRAPLVRSKLVQLEEKRHLLLIDM
HHIITDGSSTGILIGDLAKIYQGADLELPQIHYKDYAVWHKEQTNYQKDEEYWLDVFKGE
LPILDLPADFERPAERSFAGERVMFGLDKQITAQIKSLMAETDTTMYMFLLAAFNVLLSKY
ASQDDIIVGSPTAGRTHPDLQGVPGMFVNTVALRTAPAGDKTFAQFLEEVKTASLQAFEH
QSYPLEELIEKLPLTRJSTSRSPLFSVMFNMQNMEIPSLRLGDLKISSYSMLHHVAKFDLSLE
AVEREEDIGLSFDYATALFKDETIRRWSRHFVNIIKAAAANPNVRLSDVDLLSSAETAALL
EERHMTQITEATFAALFEKQAQQTPDHSAVKAGGNLLTYRELDEQANQLAHHLRAQGAG
NEDIVAIVMDRSAEVMVSILGVMKAGAAFLPIDPDTPEERIRYSLEDSGAKFAVVNERNM
TAIGQYEGIIVSLDDGKWRNESKERPSSISGSRNLAYVIYTSGTTGKPKGVQIEHRNLTNYV
SWFSEEAGLTENDKTVLLSSYAFDLGYTSMFPVLLGGGELHIVQKETYTAPDEIAHYIKEH
GITYKLTPSLFHTIVNTASFAKDANFESLRLIVLGGEKIIPTDVIAFRKMYGHTEFINHYGPT
EATIGAIAGRVDLYEPDAFAKRPTIGRPIANAGALVLNEALKLVPPGASGQLYITGQGLAR
GYLNRPQLTAERFVENPYSPGSLMYKTGDVVRRLSDGTLAFIGRADDQVKIRGYRIEPKEI
ETVMLSLSGIQEAVVLAVSEGGLQELCAYYTSDQDIEKAELRYQLSLTLPSHMIPAFFVQV
DAIPLTANGKTDRNALPKPNAAQSGGKALAAPETALEESLCRIWQKTLGIEAIGHDDNFFD
LGGHSLKGMMLIANIQAELEKSVPLKALFEQPTVRQLAAY
>SrfAB-M6

SEQ ID NO: 7

LSSAQKRMYVLNQLDRQTISYNMPSVLLMEGELDISRLRDSLNQLVNRHESLRTSFMEAN
GEPVQRIIEKAEVDLHVFEAKEDEADQKIKEFIRPFDLNDAPLIRAALLRIEAKKHLLLLDM
HHIIADGVSRGIFVKELALLYKGEQLPEPTLHYKDFAVWQNEAEQKERMKEHEAYWMSV
LSGELPELDLPLDYARPPVQSFKGDTIRFRTGSETAKAVEKLLAETGTTLHMVLHAVFHVF
LSKISGQRDIVIGSVTAGRTNADVQDMPGMFVNTLALRMEAKEQQTFAELLELAKQTNLS
ALEHQEYPFEDLVNQLDLPRDMSRNPLFNVMVTTENPDKEQLTLQNLSISPYEAHQGTSK
FDLTLGGFTDENGIGLQLEYATDLFAKETAEKWSEYVLRLLKAVADNPNQPLSSLLLVTE
TEKQALLEAWKGKALPVPTDKTVHQLFEETVQRHKDRPAVTYNGQSWTYGELNAKANR
LARILMDCGISPDDRVGVLTKPSLEMSAAVLGVLKAGAAFVPIDPDYPDQRIEYILQDSGA
KLLLKQEGISVPDSYTGDVILLDGSRTILSLPLDENDEGNPETAVTAENLAYMIYTSGTTGQ
PKGVMVEHHALVNLCFWHHDAFSMTAEDRSAKYAGFGFDASIWEMFPTWTIGAELHVID
EAIRLDIVRLNDYFETNGVTITFLPTQLAEQFMELENTSLRVLLTGGDKLKRAVKKPYTLV
NNYGPTENTVVATSAEIHPEEGSLSIGRAIANTRVYILGEGNQVQPEGVAGELCVAGRGLA
RGYLNREDETAKRFVADPFVPGERMYRTGDLVKWVNGGIEYIGRIDQQVKVRGYRIELSE
IEVQLAQLSEVQDAAVTAVKDKGGNTAIAAYVTPETADIEALKSTLKETLPDYMIPAFWV
TLNELPVTANGKVDRKALPEPDIEAGSGEYKAPTTDMEELLAGIWQDVLGMSEVGVTDN
FFSLGGDSIKGIQMASRLNQHGWKLEMKDLFQHPTIEELTQYV
>SrfAC-M7 (C-A)

SEQ ID NO: 8

LSPMQEGMLFHAILNPGQSFYLEQITMKVKGSLNIKCLEESMNVIMDRYDVFRTVFIHEKV
KRPVQVVLKKRQFHIEEIDLTHLTGSEQTAKINEYKEQDKIRGFDLTRDIPMRAAIFKKAEE
SFEWVWSYHHIILDGWCFGIVVQDLFKVYNALREQKPYSLPPVKPYKDYIKWLEKQDKQ
ASLRYWREYLEGFEGQTTFAEQRKKQKDGYEPKELLFSLSEAETKAFTELAKSQHTTLST
ALQAVWSVLISRYQQSGDLAFGTVVSGRPAEIKGVEHMVGLFINVVPRRVKLSEGITFNG
LLKRLQEQSLQSEPHQYVPLYDIQSQADQPKLIDHIIVFENYPLQDAKNEESSENGFDMVD
VHVFEKSNYDLNLMASPGDEMLIKLAYNENVFDEAFILRLKSQLLTAIQQLIQNPDQPVST
INLVDDREREFLLTGLNPPAQAHETKPLTYWFKEAVNANPDAPALTYSGQTLSYRELDEE
ANRIARRLQKHGAGKGSVVALYTKRSLELVIGILGVLKAGAAYLPVDPKLPEDRISYMLA
DSAAACLLTHQEMKEQAAELPYTGTTLFIDDQTRFEEQASDPATAIDPNDPAYIMYTSGTT
GKPKGNITTHANIQGLVKHVDYMAFSDQDTFLSVSNYAFDAFTFDFYASMLNAARLIIAD
EHTLLDTERLTDLILQENVNVMFATTALFNLLTDAGEDWMKGLRCILFGGERASVPHVRK
ALRIMGPGKLINCYGPTEGTVFATAHVVHDLPDSISSLPIGKPISNASVYILNEQSQLQPFGA
VGELCISGMGVSKGYVNRADLTKEKFIENPFKPGETLYRTGDLARWLPDGTIEYAGRIDD
QVKIRGHRIELEEIEKQLQEYPGVKDAVVVADRHESGDASINAYLVNRTQLSAEDVKAHL
KKQLPAYMVPQTFTFLDELPLTTNGKVNKRLLPKPDQDQLAEEWIGPRNEMEETIAQIWS
EVLGRKQIGIHDDFFALGGHSLKAMTAASRIKKELGIDLPVKLLFEAPTIAGISAYL

SEQ ID NO: 9

KEKVISKWNETAKSEKLVSLQDMFEKQAVLTPERIALMCDDIQVNYRKLNEEANRLARLL
IEKGIGPEQFVALALPRSPEMVASMLGVLKTGAAYLPLDPEFPADRISYMLEDAKPSCIITT
EEIAASLPDDLAVPELVLDQAVTQEIIKRYSPENQDVSVSLDHPAYIIYTSGSTGRPKGVVV
TQKSLSNFLLSMQEAFSLGEEDRLLAVTTVAFDISALELYLPLISGAQIVIAKKETIREPQAL
AQMIENFDINIMQATPTLWYPIALVTSEPEKLRGLRVLVGGEALPSGLLQELQDLHCSVTNL
YGPTETTIWSAAAFLEEGLKGVPPIGKPIWNTQVYVLDNGLQPVPPGVVGELYIAGTGLAR
GYFHRPDLTAERFVADPYGPPGTRMYRTGDQARWRADGSLDYIGRADHQIKIRGFRIELG
EIDAVLANHPHIEQAAVVVREDQPGDKRLAAYVVADAAIDTAELRRYMGASLPDYMVPS
AFVEMDELPLTPNGKLDRKALPAPDFSTSVSDRA
dhbF

SEQ ID NO: 10

MPDTKDLQYSLTGAQTGIWFAQQLDPDNPIYNTAEYIEINGPVNIALFEEALRHVIKEAESL
HVRFGENMDGPWQMINPSPDVQLHVIDVSSEPDPEKTALNWMKADLAKPVDLGYAPLFN
EALFIAGPDRFFWYQRIHHIAIDGFGFSLIAQRVASTYTALIKGQTAKSRSFGSLQAILEEDT
DYRGSEQYEKDRQFWLDRFADAPEVVSLADRAPRTSNSFLRHTAYLPPSDVNALKEAAR
YFSGSWHEVMIAVSAVYVHRMTGSEDVVLGLPMMGRIGSASLNVPAMVMNLLPLRLTV
SSSMSFSELIQQISREIRSIRRHHKYRHEELRRDLKLIGENHRLFGPQINLMPFDYGLDFAGV
RGTTHNLSAGPVDDLSINVYDRTDGSGLRIDVDANPEVYSESDIKLHQQRILQLLQTASAG
EDMLIGQMELLLPEEKEKVISKWNETAKSEKLVSLQDMFEKQAVLTPERIALMCDDIQVN
YRKLNEEANRLARLLIEKGIGPEQFVALALPRSPEMVASMLGVLKTGAAYLPLDPEFPAD
RISYMLEDAKPSCIITTEEIAASLPDDLAVPELVLDQAVTQEIIKRYSPENQDVSVSLDHPAY
IIYTSGSTGRPKGVVVTQKSLSNFLLSMQEAFSLGEEDRLLAVTTVAFDISALELYLPLISGA
QIVIAKKETIREPQALAQMIENFDINIMQATPTLWHALVTSEPEKLRGLRVLVGGEALPSGL
LQELQDLHCSVTNLYGPTETTIWSAAAFLEEGLKGVPPIGKPIWNTQVYVLDNGLQPVPPG
VVGELYIAGTGLARGYFHRPDLTAERFVADPYGPPGTRMYRTGDQARWRADGSLDYIGR
ADHQIKIRGFRIELGEIDAVLANHPHIEQAAVVVREDQPGDKRLAAYVVADAAIDTAELR
RYMGASLPDYMVPSAFVEMDELPLTPNGKLDRKALPAPDFSTSVSDRAPRTPQEEILCDLF
AEVLGLARVGIDDSFFELGGHSLLAARLMSRIREVMGAELGIAKLFDEPTVAGLAAHLDL
AQSACPALQRAERPEKIPLSFAQRRLWFLHCLEGPSPTYNIPVAVRLSGELDQGLLKAALY
DLVCRHESLRTIFPESQGTSYQHILDADRACPELHVTEIAEKELSDRLAEAVRYSFDLAAEP
AFRAELFVIGPDEYVLLLLVHHIVGDGWSLTPLTRDLGTAYAARCHGRSPEWAPLAVQYA
DYALWQQELLGNEDDPNSLIAGQLAFWKETLKNLPDQLELPTDYSRPAEPSHDGDTIHFRI
EPEFHKRLQELARANRVSLFMVLQSGLAALLTRLGAGTDIPIGSPIAGRNDDALGDLVGLF
INTLVLRTDTSGDPSFRELLDRVREVNLAAYDNQDLPFERLVEVLNPARSRATHPLFQIML
AFQNTPDAELHLPDMESSLRINSVGSAKFDLTLEISEDRLADGTPNGMEGLLEYSTDLFKR
ETAQALADRLMRLLEAAESDPDEQIGNLDILAPEEHSSMVTDWQSVSEKIPHACLPEQFEK
QAALRPDAIAVVYENQELSYAELNERANRLARMMISEGVGPEQFVALALPRSLEMAVGL
LAVLKAGAAYLPLDPDYPADRIAFMLKDAQPAFIMTNTKAANHIPPVENVPKIVLDDPEL
AEKLNTYPAGNPKNKDRTQPLSPLNTAYVIYTSGSTGVPKGVMIPHQNVTRLFAATEHWF
RFSSGDIWTMFHSYAFDFSVWEIWGPLLHGGRLVIVPHHVSRSPEAFLRLLVKEGVTVLN
QTPSAFYQFMQAEREQPDLGQALSLRYVIFGGEALELSRLEDWYNRHPENRPQLINMYGI
TETTVHVSYIELDRSMAALRANSLIGCGIPDLGVYVLDERLQPVPPGVAGELYVSGAGLA
RGYLGRPGLTSERFIADPFGPPGTRMYRTGDVARLRADGSLDYVGRADHQVKIRGFRIEL
GEIEAALVQHPQLEDAAVIVREDQPGDKRLAAYVIPSEETFDTAELRRYAAERLPDYMVP
AAFVTMKELPLTPNGKLDRKALPAPDFAAAVTGRGPRTPQEEILCDLFMEVLHLPRVGID
DRFFDLGGHSLLAVQLMSRIREALGVELSIGNLFEAPTVAGLAERLEMGSSQSALDVLLPL
RTSGDKPPLFCVHPAGGLSWCYAGLMTNIGTDYPIYGLQARGIGQREELPKTLDDMAAD
YIKQIRTVQPKGPYHLLGWSLGGNVVQAMATQLQNQGEEVSLLVMLDAYPNHFLPIKEA
PDDEEALIALLALGGYDPDSLGEKPLDFEAAIEILRRDGSALASLDETVILNLKNTYVNSVG
ILGSYKPKTFRGNVLFFRSTIIPEWFDPIEPDSWKPYINGQIEQIDIDCRHKDLCQPEPLAQIG
KVLAVKLEELNK

SEQ ID NO: 11

EKQMILKTWNATGKTYPYITFHELFEQQAKKTPDRAAVSYEGQTLTYRELDEKSTQLAIY
LQAHGVGPDRLAGIYVDRSLDMLVGLLAILKAGGAYVPLDPSYPAERLEYMLEDSEVFIT
LTTSELVNTLSWNGVTTALLDQDWDEIAQTASDRKVLTRTVTPENLAYVIYTSGSTGKPK
GVMIPHKALTNFLVSMGETPGLTAEDKMLAVTTYCFDIAALELFLPLIKGAHCYICQTEHT
KDVEKLKRDIRAIKPTVMQATPATWKMLFYSGWENEESVKILCGGEALPETLKRYFLDTG
SEAWNMFGPTETTIWSAVQRINVECSHATIGRPIANTQIYITDSQLAPVPAGVPGELCIAGD
GVAKGYYKKEELTDSRFIDNPFEPGSKLYRTGDMARWLTGGRIEYIGRIDNQVKIRGFRIE
LGDIESRLSEHPGILECVVVADMDNLAAYYTAKHANASLTARELRHFVKNALPAYMVPS
YFIQLDHMPLTPNGKIDRNSLKNIDLSGEQLKQRQTS
Pksj (BSU17180)

SEQ ID NO: 12

MRNNDNIRILTNPSVSHGEPLHISEKQPATIPEVLYRTATELGDTKGIIYLQPDGTEVYQSY
RRLWDDGLRIAKGLRQSGLKAKQSVILQLGDNSQLLPAFWGCVLTGVVPAPLAVPPTYA
ESSSGTQKLKDAWTLLDKPAVITDRGMHQEMLDWAKEQGLEGFRAIIVEDLLSAEADTD
WHQSSPEDLALLLLTSGStGTPKAVMLNHRNIMSMVKGIIQMQGFTREDITFNWMPFDHV
GGIGMLHLRDVYLGCQEINVSSETILMEPLKWLDWIDHYRASVTWAPNFAFGLVTDFAEE
IKDKXWDLSSMRYMLNGGEAMVAKVGRRILELLEPHGLPADAIRPAWGMSETSSGVIFS
HEFTRAGTSDDDHFVEIGSPIPGFSMRIVNDHNELVEEGEIGRFQVSGLSVTSGYYQRPDLN
ESVFTEDGWFETGDLGFLRNGRLTITGRTKDAIIINGINYYSHAIESAVEELPEIETSYTAAC
AVRLGQNSTDQLAIFFVTSAKLNDEQMSQLLRNIQSHVSQVIGVTPEYLLPVQKEEIPKTAI
GKIQRTQLKTSFENGEFDHLLHKPNRMNDAVQDEGIQQADQVKRVREEIQKHLLTCLTEE
LHVSHDWVEPNANIQSLGVNSIKMMKLIRSIEKNYHIKLTAREIHQYPTIERLASYLSEHED
LSSLSADKKGTDTYKTEPERSQATFQPLSEVQKGLWTLQKMSPEKSAYHVPLCFKFSSGL
HHETFQQAFGLVLNQHPILKHVIQEKDGVPFLKNEPALSIEIKTENISSLKESDIPAFLRKKV
KEPYVKENSPLVRVMSFSRSEQEHFLLVVIHHLIFDGVSSVTFIRSLFDTYQLLLKGQQPEK
AVSPAIYHDFAAWEKNMLAGKDGVKHRTYWQKQLSGTLPNLQLPNVSASSVDSQFRED
TYTRRLSSGFMNQVTFAKEHSVNVTTVFLSCYMMLLGRYTGQKEQIVGMPAMVRPEER
FDDAIGHFLhMLPIRSELNPAJDTFSSFISKLQLTILDGLDHAAYPFPKMVRDLNIPRSQAGSP
VFQTAFFYQNFLQSGSYQSLLSRYADFFSVDFVEYIHQEGEYELVFELWETEEKMELNIKY
NTGLFDAASISAMFDHFVYVTEQAMLNPSQPLKEYSLLPEAEKQMILKTWNATGKTYPYI
TFHELFEQQAKKTPDRAAVSYEGQTLTYRELDEKSTQLAIYLQAHGVGPDRLAGIYVDRS
LDMLVGLLAILKAGGAYVPLDPSYPAERLEYMLEDSEVFITLTTSELVNTLSWNGVTTAL
LDQDWDEIAQTASDRKVLTRTVTPENLAYVIYTSGSTGKPKGVMIPHKALTNFLVSMGET
PGLTAEDKMLAVTTYCFDIAALELFLPLIKGAHCYICQTEHTKDVEKLKRDIRAIKPTVMQ
ATPATWKMLFYSGWENEESVKILCGGEALPETLKRYFLDTGSEAWNMFGPTETTIWSAV
QRINVECSHATIGRPIANTQIYITDSQLAPVPAGVPGELCIAGDGVAKGYYKKEELTDSRFI
DNPFEPGSKLYRTGDMARWLTGGRIEYIGRIDNQVKIRGFRIELGDIESRLSEHPGILECVV
VADMDNLAAYYTAKHANASLTARELRHFVKNALPAYMVPSYFIQLDHMPLTPNGKIDRN
SLKNIDLSGEQLKQRQTSPKNIQDTVFTIWQEVLKTSDIEWDDGFFDVGGDSLLAVTVAD
RIKHELSCEFSVTDLFEYSTIKNISQYITEQRMGDASDHIPTDPAAHIEDQSTEMSDLPDYYD
DSVAIIGISCEFPGAKNHDEFWENLRDGKESIAFFNKEELQRFGISKEIAENADYVPAKASID
GKDRFDPSFFQISPKDAEFMDPQLRMLLTHSWKAIEDAGYAARQIPQTSVFMSASNNSYRV
ALLPSDTTESLETPDGYVSWVLAQSGTIPTMISHKLGLRGPSYFVHANCSSSLIGLHSAYKS
LLSGESDYALVGGATLHTESNIGYVHQPGLNFSSDGHIKAFDASADGMIGGEGVAVVLLK
KAADAVKDGDHIYALLRGIGVNNDGADKVGFYAPSVKGQADVVQQVMNQTKVQPESIC
YVEAHGTGTKLGDPIELAALTNVYRQYTNKTQFCGIGSVKTNIGHLDTAAGLAGCIKVVM
SLYHQELAPSVNYKEPNPNTDLASSPFYVVDQKKTLSREIKTHRAALSSFGLGGTNTHAIF
EQFKRDSDKGKIDGTCIVPISAKNKERLQEYAEDILAYLERRGFENSQLPDFAYTLQVGRE
AMEHRVVFIADHVNELKQRLTDFINGNTAIEGCFQGSKHNAREVSWLTEDEDSAELIRKW
MAKGKVNKLAEMWSKGAHIDWMQLYKGERPNRMSLPTYPFAKERYWPSQDDRKPVAQ
ISGNQTGIGSIHPLLHQNTSDFSEQKFSSVFTGDEFFLRDHVVRGKPVLPGVAYLEMAYAAI
NQAAGSEIGQDVRIRLNHTVWVQPVVVDRHSAQVDISLFPEEDGKITFDIYSTQEDGDDPV
IHSQGSAELASAAETPVADLTEMSRRCGKGKMSPDQFYEEGRSRGMFHGPAFQGIKNVNI
GNREVLAQLQLPEIVSGTNEQFVLHPSIMDSALQTATICIMQELTDQKLILPFALEELEVIKG
CSSSMWAYARLSDSDHSGGVVQKADIDVIDESGTVCVRIKGFSTRVLEGEVHTSKPSTRH
ERLMLEPVWEKQNEEREDEDLSYTEHIIVLFETERSVTDSIASHMKDARVITLNEAVGHIA
ERYQCYMQNIFELLQSKVRKLSAGRIIIQAIVPLEKEKQLFAGVSGLFKTAEIEFSKLTAQVI
EIEKPEEMIDLHLKLKDDSRRPFDKQIRYEAGYRFVKGWREMVLPSADTLHMPWRDEGV
YLITGGAGSLGLLFAKEIANRTGRSTIVLTGRSVLSEDKENELEALRSIGAEVVYREADVSD
QHAVRHLLEEIKERYGTLNGIIHGAGSSKDRFIIHKTNEEFQEVLQPKVSGLLHVDECSKDF
PLDFFIFFSSVSGCLGNAGQADYAAANSFMDAFAEYRRSLAASKKRFGSTISFNWPLWEE
GGMQVGAEDEKRMLKTTGMVPMPTDSGLKAFYQGIVSDKPQVFVMEGQLQKMKQKLL
SAGSKAKRNDQRKADQDQGQTRKLEAALIQMVGAILKVNTDDIDVNTELSEYGFDSVTF
TVFTNKINEKFQLELTPTIFFEYGSVQSLAEYVVAAYQGEWNQDATAKGKDERTNLVHSL
SSLEASLSNMVSAILKVNSEDIDVNTELSEYGFDSVTFTVFTNKINEEFQLELTPTIFFEYGS
LHSLAEYLTVEHGDTLVQEREKPEGQEELQTKSSEAPKITSRRKRRFTQPIIKAERNKKQ
AADFEPVAIVGISGRFPGAMDIDEFWKNLEEGKDSITEWKDRWDWREHYGNPDTDVNK
TDIKWGGFIDGVAEFDPLFFGISPREADYVDPQQRLLMTYVWKALEDAGCSPQSLSGTGT
GIFIGTGNTGYKDLFHRANLPIEGHAATGHMIPSVGPNRMSYFLNIHGPSEPVETACSSSLV
AIHRAVTAMQNGDCEMAIAGGVNTILTEEAHISYSKAGMLSTDGRCKTFSADANGYVRG
EGVGMVMLKKLEDAERDGNHIYGVIRGTAENHGGRANTLTSPNPKAQADLLVRAYRQA
DIDPSTVTYIEAHGTGTELGDPIEINGLKAAFKELSNMRGESQPDVPDHRCGIGSVKSNIGH
LELAAGISGLIKVLLQMKHKTLVKSLHCETLNPYLQLTDSPFYIVQEKQEWKSVTDRDGN
ELPRRAGISSFGIGGVNAHIVIEEYNIPKANSEHTATEQPNVIVLSAKNKSRLIDRASQLLEV
RNKKYTDQDLHRIAYTLQVGREEMDERLACVAGTMQELEEKLQAFVDGKEETDEFFRGQ
SHRNKETQTIFTADEDMALALDAWIRKRKYAKLADLWVKGVSIQWNTLYGETKPRLISLP
SYPFAIQDHYWVPAKEHSERDKKELVNAIEDRAACFLTKQWSLSPIGSAVPGTRTVAILCC
QETADLAAEVSSYFPNHLLIDVSRIENDQSDIDWKEFDGLVDVIGCGWDDEGRLDWIEWV
QRLVEFGHKEGLRLLCVTKGLESFQNTSVRMAGASRAGLYRMLQCEYSHLISRHMDAEE
VTDHRRLAKLIADEFYSDSYDAEVCYRDGLRYQAFLKAHPETGKATEQSAVFPKDHVLLI
TGGTRGIGLLCARHFAECYGVKKLVLTGREQLPPREEWARFKTSNTSLAEKJQAVRELEA
KGVQVEMLSLTLSDDAQVEQTLQHIKRTLGPIGGVIHCAGLTDMDTLAFIRKTSDDIQRVL
EPKVSGLTTLYRHVCNEPLQFFVLFSSVSAIIPELSAGQADYAMANSYMDYFAEAHQKHA
PIISVQWPNWKETGMGEVTNQAYRDSGLLSITNSEGLRFLDQIVSKKFGPVVLPAMANQT
NWEPELLMKRRKPHEGGLQEAALQSPPARDIEEADEVSKCDGLLSETQSWLIDLFTEELRI
DREDFEIDGLFQDYGVDSIILAQVLQRINRKLEAALDPSILYEYPTIQRFADWLIGSYSERLS
ALFGGRISDASAPLENKIEAEASVPGKDRALTPQIQAPAILSPDSHAEGIAVVGLSCRFPGAE
TLESYWSLLSEGRSSIGPIPAERWGCKTPYYAGVIDGVSYFDPDFFLLHEEDVRAMDPQAL
LVLEECLKLLYHAGYTPEEIKGKPVGVYIGGRSQHKPDEDSLDHAKNPIVTVGQNYLAAN
LSQFFDVRGPSVVVDTACSSALVGMNMAIQALRGGDIQSAIVGGVSLLSSDASHRLFDRR
GILSKHSSFHVFDERADGVVLGEGVGMVMLKTVKQALEDGDIIYAVVKAASVNNDGRTA
GPATPNLEAQKEVMKDALFKSGKKPEDISYLEANGSGSIVTDLLELKAIQSVYRSGHSSPL
SLGSIKPNIGHPLCAEGIASFIKVVLMLKERRFVPFLSGEKEMAHFDQQKANITFSRALEKW
TDSQPTAAINCFADGGTNAHVIVEAWEKDEKHAIKRSPISPPQLKKRMLSPGEPKLEAETS
KMTAANIWDTYEVEV

SEQ ID NO: 13

cctttaacggatgcacaaaaacgaatttggtacacagaaaaattttatcctcacacgagcatttcaaatcttgcggggattggtaagctggtttcagc
tgatgcgattgattatgtgcttgttgagcaggcgattcaagagtttattcgcagaaatgacgccatgcgccttcggttgcggctagatgaaaacgg
ggagcctgttcaatatattagcgagtatcggcctgttgatataaaacatactgacactactgaagatccgaatgcgatagagtttatttcacaatgga
gccgggaggaaacgaagaaacctttgccgctatacgattgtgatttgttccgtttttccttgttcaccataaaggaaaatgaagtgtggttttacgca
aatgttcatcacgtgatttctgatggtatctccatgaatattctcgggaatgcgatcatgcacatttatttagaattagccagcggctcagagacaaaa
gaaggaatctcgcattcatttatcgatcatgttttatctgaacaggaatatgctcaatcgaagcggtttgaaaaggacaaggcgttttggaacaaac
aatttgaatcggtgcctgaacttgtttccttgaaacggaatgcatccgcagggggaagtttagatgctgagaggttctctaaagatgtgcctgaagc
gcttcatcagcagattctgtcgttttgtgaggcgaataaagtcagtgttctttcggtatttcaatcgctgctcgccgcctatttgtacagggtcagcgg
ccagaatgatgttgtgacgggaacatttatgggcaaccggacaaatgcgaaagagaagcagatgcttggcatgtttgtttctacggttccgcttcg
gacaaacattgacggcgggcaggcgttttcagaatttgtcaaagaccggatgaaggatctgatgaagacacttcgccaccaaaagtatccgtat
aatctcctaatcaacgatttgcgtgaaacaaagagctctctgaccaagctgttcacggtttctcttgaatatcaagtgatgcagtggcagaaagaag
aggatcttgcctttttgactgagccgattttcagcggcagcggattaaatgatgtctcaattcatgtaaaggatcgatgggatactgggaaactcac
catagattttgattaccgcactgatttattttcacgtgaagaaatcaacatgatttgtgagcgcatgattaccatgctggagaacgcgttaacgcatcc
agaacatacaattgatgaattaacactgatttctgatgcggagaagcagatgattctcaaaacatggaacgccacaggaaaaacgtatccatatat
aacttttcatgagttgtttgagcagcaggcgaaaaagacgcctgatagagcggctgtcagctatgaaggtcaaacattgacgtatcgggagcttg
atgagaaaagcacacagctggccatttatttgcaggcgcatggagtgggtcctgaccgtctggcggggatctatgtggatcgatcgctggacat
gctagtgggtttattagcgatcctcaaggctgggggagcgtatgtgccgctagacccgtcctatccggctgaacgattagaatacatgcttgagg
acagtgaagttttcattacactgacgacatcggaattagtaaatacgttgagttggaacggtgtcacaacagcccttttagatcaagattgggatga
aattgctcaaacagcctctgatcgaaaagtgcttacacgcactgtcacgccagagaacttggcatatgtcatttatacatccggcagcacaggaaa
gccaaaaggtgtcatgataccacataaagctttgacaaactttctcgtttcgatgggggaaacaccaggtcttacggcagaggataaaatgcttgc
tgtcacaacctactgttttgatattgcagctctggaattatttttgcctttaataaagggcgcacactgctatatttgtcaaacggagcatacaaaagac
gttgaaaaactgaaacgggacatccgcgcgatcaaaccgacagtgatgcaggcaacccccgctacgtggaagatgctcttttattcaggctgg
gaaaatgaagagagcgtgaaaattttatgcggtggcgaagcattgcctgagacattaaaacgatatttcttagatacgggcagcgaagcctgga
atatgttcgggccaaccgaaacaactatctggtcagcggttcagcgcattaacgttgaatgctctcatgccacgataggaaggccaatcgccaat
acacaaatctatattacggattctcagctcgcgccagtgccggcaggtgttccgggtgagctgtgcattgcaggagacggtgtggcgaagggct
actacaaaaaggaagaattaacggattcgagattcattgacaacccttttgagcctgggtctaagctttatagaacgggagacatggcccgttggc
ttacgggagggcgaattgaatatataggccgcatcgataatcaagtaaaaatccgcggattccgtattgaacttggtgatattgaaagcaggctta
gtgagcatcccggcattctggaatgcgttgtggtcgcagatatggataacctagctgcctattatacagctaaacatgcaaatgcttctctcacagc
gagagagctgcgtcattttgtgaaaaacgctttgcctgcctatatggtgccttcttattttattcagcttgatcatatgccgttaactccgaacggaaag
atagatagaaacagccttaagaatatcgatttatcaggggagcagctaaagcaaaggcagacctctcctaagaacattcaggatactgtttttacc
atttggcaggaagtgctgaaaacgagtgacattgaatgggatgacttctttgcgctcggagggcattccttgaaggccatgaccgccgcgtccc
gcatcaagaaagagctcgggattgatcttccagtgaagcttttgtttgaagcgccgacgatcgccggcatttcagcgtatttgaaaaacgggggct
ctgatggcttgcaggatgtaacgataatgaatcaggatcaggagcagatcattttcgcatttccgccggttctgggctatggccttatgtaccaaaa
tctgtccagccgcttgccgtcatacaagctatgcgcctttgattttattgaggaggaagaccggcttgaccgctatgcggatttgatccagaagctg
cagccggaagggcctttaacattgtttggatattcagcgggatgcagcctggcgtttgaagctgcgaaaaagcttgaggaacaaggccgtattgt
tcagcggatcatcatggtggattcctataaaaaacaaggtgtcagtgatctggacggacgcacggttgaaagtgatgtcgaagcgttgatgaatg
tcaatcgggacaatgaagcgctcaacagcgaagccgtcaaacacggcctcaagcaaaaaacacatgccttttactcatactacgtcaacctgat
cagcacaggccaggtgaaagcagatattgatctgttgacttccggcgctgattttgacatgccggaatggcttgcatcatgggaagaagctacaa
caggtgtttaccgtgtgaaaagaggcttcggaacacacgcagaaatgctgcagggcgaaacgctagataggaatgcggagattttgctcgaatt
tcttaatacacaaaccgtaacggtttcataa

SEQ ID NO: 14

cctttaacggatgcacaaaaacgaatttggtacacagaaaaattttatcctcacacgagcatttcaaatcttgcggggattggtaagctggtttcagc
tgatgcgattgattatgtgcttgttgagcaggcgattcaagagtttattcgcagaaatgacgccatgcgccttcggttgcggctagatgaaaacgg
ggagcctgttcaatatattagcgagtatcggcctgttgatataaaacatactgacactactgaagatccgaatgcgatagagtttatttcacaatgga
gccgggaggaaacgaagaaacctttgccgctatacgattgtgatttgttccgtttttccttgttcaccataaaggaaaatgaagtgtggttttacgca
aatgttcatcacgtgatttctgatggtatctccatgaatattctcgggaatgcgatcatgcacatttatttagaattagccagcggctcagagacaaaa
gaaggaatctcgcattcatttatcgatcatgttttatctgaacaggaatatgctcaatcgaagcggtttgaaaaggacaaggcgttttggaacaaac
aatttgaatcggtgcctgaacttgtttccttgaaacggaatgcatccgcagggggaagtttagatgctgagaggttctctaaagatgtgcctgaagc
gcttcatcagcagattctgtcgttttgtgaggcgaataaagtcagtgttctttcggtatttcaatcgctgctcgccgcctatttgtacagggtcagcgg
ccagaatgatgttgtgacgggaacatttatgggcaaccggacaaatgcgaaagagaagcagatgcttggcatgtttgtttctacggttccgcttcg
gacaaacattgacggcgggcaggcgttttcagaatttgtcaaagaccggatgaaggatctgatgaagacacttcgccaccaaaagtatccgtat
aatctcctaatcaacgatttgcgtgaaacaaagagctctctgaccaagctgttcacggtttctcttgaatatcaagtgatgcagtggcagaaagaag
aggatcttgcctttttgactgagccgattttcagcggcagcggattaaatgatgtctcaattcatgtaaaggatcgatgggatactgggaaactcac
catagattttgattaccgcactgatttattttcacgtgaagaaatcaacatgatttgtgagcgcatgattaccatgctggagaacgcgttaacgcatcc
agaacatacaattgatgaattaacactgatttctgatgcggagaagcagatgattctcaaaacatggaacgccacaggaaaaacgtatccatatat
aacttttcatgagttgtttgagcagcaggcgaaaaagacgcctgatagagcggctgtcagctatgaaggtcaaacattgacgtatcgggagcttg
atgagaaaagcacacagctggccatttatttgcaggcgcatggagtgggtcctgaccgtctggcggggatctatgtggatcgatcgctggacat
gctagtgggtttattagcgatcctcaaggctgggggagcgtatgtgccgctagacccgtcctatccggctgaacgattagaatacatgcttgagg
acagtgaagttttcattacactgacgacatcggaattagtaaatacgttgagttggaacggtgtcacaacagcccttttagatcaagattgggatga
aattgctcaaacagcctctgatcgaaaagtgcttacacgcactgtcacgccagagaacttggcatatgtcatttatacatccggcagcacaggaaa
gccaaaaggtgtcatgataccacataaagctttgacaaactttctcgtttcgatgggggaaacaccaggtcttacggcagaggataaaatgcttgc
tgtcacaacctactgttttgatattgcagctctggaattatttttgcctttaataaagggcgcacactgctatatttgtcaaacggagcatacaaaagac
gttgaaaaactgaaacgggacatccgcgcgatcaaaccgacagtgatgcaggcaacccccgctacgtggaagatgctcttttattcaggctgg
gaaaatgaagagagcgtgaaaattttatgcggtggcgaagcattgcctgagacattaaaacgatatttcttagatacgggcagcgaagcctgga
atatgttcgggccaaccgaaacaactatctggtcagcggttcagcgcattaacgttgaatgctctcatgccacgataggaaggccaatcgccaat
acacaaatctatattacggattctcagctcgcgccagtgccggcaggtgttccgggtgagctgtgcattgcaggagacggtgtggcgaagggct
actacaaaaaggaagaattaacggattcgagattcattgacaacccttttgagcctgggtctaagctttatagaacgggagacatggcccgttggc
ttacgggagggcgaattgaatatataggccgcatcgataatcaagtaaaaatccgcggattccgtattgaacttggtgatattgaaagcaggctta
gtgagcatcccggcattctggaatgcgttgtggtcgcagatatggataacctagctgcctattatacagctaaacatgcaaatgcttctctcacagc
gagagagctgcgtcattttgtgaaaaacgctttgcctgcctatatggtgccttcttattttattcagcttgatcatatgccgttaactccgaacggaaag
atagatagaaacagccttaagaatatcgatttatcaggggagcagctaaagcaaaggcagacctctcctaagaacattcaggatactgtttttacc
atttggcaggaagtgctgaacgttgagaaggcggggatctttgacttctttgcgctcggagggcattccttgaaggccatgaccgccgcgtcccg
catcaagaaagagctcgggattgatcttccagtgaagcttttgtttgaagcgccgacgatcgccggcatttcagcgtatttgaaaaacgggggctc
tgatggcttgcaggatgtaacgataatgaatcaggatcaggagcagatcattttcgcatttccgccggttctgggctatggccttatgtaccaaaat
ctgtccagccgcttgccgtcatacaagctatgcgcctttgattttattgaggaggaagaccggcttgaccgctatgcggatttgatccagaagctgc
agccggaagggcctttaacattgtttggatattcagcgggatgcagcctggcgtttgaagctgcgaaaaagcttgaggaacaaggccgtattgtt
cagcggatcatcatggtggattcctataaaaaacaaggtgtcagtgatctggacggacgcacggttgaaagtgatgtcgaagcgttgatgaatgt
caatcgggacaatgaagcgctcaacagcgaagccgtcaaacacggcctcaagcaaaaaacacatgccttttactcatactacgtcaacctgatc
agcacaggccaggtgaaagcagatattgatctgttgacttccggcgctgattttgacatgccggaatggcttgcatcatgggaagaagctacaac
aggtgtttaccgtgtgaaaagaggcttcggaacacacgcagaaatgctgcagggcgaaacgctagataggaatgcggagattttgctcgaattt
cttaatacacaaaccgtaacggtttcataa

SEQ ID NO: 15

cctttaacggatgcacaaaaacgaatttggtacacagaaaaattttatcctcacacgagcatttcaaatcttgcggggattggtaagctggtttcagc
tgatgcgattgattatgtgcttgttgagcaggcgattcaagagtttattcgcagaaatgacgccatgcgccttcggttgcggctagatgaaaacgg
ggagcctgttcaatatattagcgagtatcggcctgttgatataaaacatactgacactactgaagatccgaatgcgatagagtttatttcacaatgga
gccgggaggaaacgaagaaacctttgccgctatacgattgtgatttgttccgtttttccttgttcaccataaaggaaaatgaagtgtggttttacgca
aatgttcatcacgtgatttctgatggtatctccatgaatattctcgggaatgcgatcatgcacatttatttagaattagccagcggctcagagacaaaa
gaaggaatctcgcattcatttatcgatcatgttttatctgaacaggaatatgctcaatcgaagcggtttgaaaaggacaaggcgttttggaacaaac
aatttgaatcggtgcctgaacttgtttccttgaaacggaatgcatccgcagggggaagtttagatgctgagaggttctctaaagatgtgcctgaagc
gcttcatcagcagattctgtcgttttgtgaggcgaataaagtcagtgttctttcggtatttcaatcgctgctcgccgcctatttgtacagggtcagcgg
ccagaatgatgttgtgacgggaacatttatgggcaaccggacaaatgcgaaagagaagcagatgcttggcatgtttgtttctacggttccgcttcg
gacaaacattgacggcgggcaggcgttttcagaatttgtcaaagaccggatgaaggatctgatgaagacacttcgccaccaaaagtatccgtat
aatctcctaatcaacgatttgcgtgaaacaaagagctctctgaccaagctgttcacggtttctcttgaatatcaagtgatgcagtggcagaaagaag
aggatcttgcctttttgactgagccgattttcagcggcagcggattaaatgatgtctcaattcatgtaaaggatcgatgggatactgggaaactcac
catagattttgattaccgcactgatttattttcacgtgaagaaatcaacatgatttgtgagcgcatgattaccatgctggagaacgcgttaacgcatcc
agaacatacaattgatgaattaacactgatttctgatgcggagaaagaaaaagtcatttctaaatggaatgagacggcaaaatccgagaagctggt
cagccttcaggacatgttcgaaaagcaggcagttcttacgccagaacgtatcgctctcatgtgtgatgacattcaagtcaactatcgaaagctcaat
gaagaggcaaaccgcctcgcgcgtctgttgatcgaaaaagggattggtccggagcaatttgtcgctttggcgctgccgcgttcccctgagatgg
tggcttcaatgcttggtgtgctcaagactggtgcggcgtatctcccccttgatccggagtttccagccgaccgcatttcttacatgctggaggatgc
gaaaccttcatgcatcataacgactgaggaaatagcagccagtctgcctgatgacctggctgtacctgagcttgtgcttgatcaggctgttacaca
ggagattataaaacgctattcgccggaaaatcaggatgtatcggtttcgcttgaccatcctgcgtatatcatctatacctcaggatcaacaggaaga
ccgaagggagtcgttgtgacacagaaaagcttaagcaattttctgctgtctatgcaggaggcattttctctaggagaagaagacaggctgttggct
gtgacgactgtcgcttttgatatttcagcattggagttatatcttccgctgattagcggagcgcaaatcgtgatcgcaaagaaagaaacgatccgtg
agccgcaggcattagctcaaatgattgaaaatttcgatattaacattatgcaggcgacaccgacactgtggcacgctttggtaacgagtgaacctg
agaaacttcgggggcttagagtgcttgtaggaggcgaggcgctgccgagcggtcttttgcaggaacttcaagaccttcattgttcagtcacgaac
ttatacggtcccactgaaacaacgatttggtctgccgcagcttttcttgaagaagggctgaagggcgttcctccgattgggaaaccgatttggaac
acgcaggtgtatgtgcttgataatggattgcagccggtgccgccgggcgttgtcggagagctttatattgcaggaaccggcttggccagaggtta
tttccatcgtcctgatttaacggcggagcgcttcgttgcagatccatacggaccgccgggaactcggatgtatcggaccggagaccaggcccgc
tggcgcgccgatgggtctttggattatatcgggcgggcggatcatcaaatcaaaattcgcggattccgaattgaacttggagaaattgatgccgtg
cttgccaatcacccgcacattgaacaggccgcggttgtcgtacgggaagatcagccgggagacaaacgattggcggcttatgtagtcgctgat
gctgctattgatactgcagagcttcgtcgttatatgggggctagtcttcctgattatatggtaccgtcggcgtttgtggagatggacgagctgccgtt
aacacctaatggcaagcttgaccggaaagcactgccggcaccagacttcagcacatctgtcagtgatcgggccccgcggactcctcaggaag
agatattgtgtgacttgtttgcagaggttctcggtttggcacgcgtcggtatcgatgacttctttgcgctcggagggcattccttgaaggccatgacc
gccgcgtcccgcatcaagaaagagctcgggattgatcttccagtgaagcttttgtttgaagcgccgacgatcgccggcatttcagcgtatttgaaa
aacgggggctctgatggcttgcaggatgtaacgataatgaatcaggatcaggagcagatcattttcgcatttccgccggttctgggctatggcctt
atgtaccaaaatctgtccagccgcttgccgtcatacaagctatgcgcctttgattttattgaggaggaagaccggcttgaccgctatgcggatttgat
ccagaagctgcagccggaagggcctttaacattgtttggatattcagcgggatgcagcctggcgtttgaagctgcgaaaaagcttgaggaacaa
ggccgtattgttcagcggatcatcatggtggattcctataaaaaacaaggtgtcagtgatctggacggacgcacggttgaaagtgatgtcgaagc
gttgatgaatgtcaatcgggacaatgaagcgctcaacagcgaagccgtcaaacacggcctcaagcaaaaaacacatgccttttactcatactac
gtcaacctgatcagcacaggccaggtgaaagcagatattgatctgttgacttccggcgctgattttgacatgccggaatggcttgcatcatgggaa
gaagctacaacaggtgtttaccgtgtgaaaagaggcttcggaacacacgcagaaatgctgcagggcgaaacgctagataggaatgcggagat
tttgctcgaatttcttaatacacaaaccgtaacggtttcataa
ME-B0014

SEQ ID NO: 16

gaattcgcatgcatcgacaaaaatgtcatgaaagaatcgttgtaagacgctcttcgcaagggtgtctttttttgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgctgttgccagaggagaaagaaaaagtcatttctaa
atggaatgagacggcaaaatccgagaagctggtcagccttcaggacatgttcgaaaagcaggcagttcttacgccagaacgtatcgctctcatg
tgtgatgacattcaagtcaactatcgaaagctcaatgaagaggcaaaccgcctcgcgcgtctgttgatcgaaaaagggattggtccggagcaatt
tgtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggaacgagatggaagaaacaatcgcacaaatatggtctgaggttctcggcagaaagcaaattggcattcatgacgat
ttctttgcgctcggagggcattccttgaaggccatgaccgccgcgtcccgcatcaagaaagagctcgggattgatcttccagtgaagcttttgtttg
aagcgccgacgatcgccggcatttcagcgtatttgaaaaacgggggctctgatggcttgcaggatgtaacgataatgaatcaggatcaggagc
agatcattttcgcatttccgccggttctgggctatggccttatgtaccaaaatctgtccagccgcttgccgtcatacaagctatgcgcctttgattttatt
gaggaggaagaccggcttgaccgctatgcggatttgatccagaagctgcagccggaagggcctttaacattgtttggatattcagcgggatgca
gcctggcgtttgaagctgcgaaaaagcttgaggaacaaggccgtattgttcagcggatcatcatggtggattcctataaaaaacaaggtgtcagt
gatctggacggacgcacggttgaaagtgatgtcgaagcgttgatgaatgtcaatcgggacaatgaagcgctcaacagcgaagccgtcaaaca
cggcctcaagcaaaaaacacatgccttttactcatactacgtcaacctgatcagcacaggccaggtgaaagcagatattgatctgttgacttccgg
cgctgattttgacatgccggaatggcttgcatcatgggaagaagctacaacaggtgtttaccgtgtgaaaagaggcttcggaacacacgcagaa
atgctgcagggcgaaacgctagataggaatgcggagattttgctcgaatttcttaatacacaaaccgtaacggtttcataaatgaagtgatgaaag
gaggagacagccaatgagccaactcttcaaatcatttgatgcgtcggaaaaaacacagctcatctgttttccgtttgccggcggctattcggcgtc
gtttcgccctctccatgcttttttgcagggggagtgcgagatgctcgctgccgagccgccgggacacggcacgaatcaaacgtcagccattgag
gatctcgaagagctgacggatttgtacaagcaagaactgaaccttcgccctgatcggccgtttgtgctgttcggacacagtatgggcggaatgat
caccttcaggctggcgcaaaagcttgagcgtgaaggcatctttccgcaggcggttatcatttctgcaatccagccgcctcatattcagcggaaga
aagtgtcccacctgcctgatgatcagtttctcgatcatattatccaattaggcggaatgcccgcagagcttgttgaaaataaggaggtcatgtcctttt
tcctgccttctttccgatcagattaccgggctcttgaacaatttgagctttacgatctggcccagatccagtcgcctgttcatgtctttaacgggcttga
tgataaaaaatgcatacgagatgcggaagggtggaagaagtgggcaaaagacatcacattccatcaatttgacggcgggcacatgttcctgctg
tcacaaacggaagaagtcgcagaacggatttttgcgatcttgaatcagcatccgatcattcaaccgtgaagccgccccgcagggcgctccgca
ggccgcttccggaccactccggaagcggccgtgcggtcggaggatcc
ME-B0015

SEQ ID NO: 17

gaattcgcatgcatcgacaaaaatgtcatgaaagaatcgttgtaagacgctcttcgcaagggtgtctttttttgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgctgttgccagaggagaaagaaaaagtcatttctaa
atggaatgagacggcaaaatccgagaagctggtcagccttcaggacatgttcgaaaagcaggcagttcttacgccagaacgtatcgctctcatg
tgtgatgacattcaagtcaactatcgaaagctcaatgaagaggcaaaccgcctcgcgcgtctgttgatcgaaaaagggattggtccggagcaatt
tgtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggactcctcaggaagagatattgtgtgacttgtttgcagaggttctcggtttggcacgcgtcggtatcgatgacgatttct
ttgcgctcggagggcattccttgaaggccatgaccgccgcgtcccgcatcaagaaagagctcgggattgatcttccagtgaagcttttgtttgaag
cgccgacgatcgccggcatttcagcgtatttgaaaaacgggggctctgatggcttgcaggatgtaacgataatgaatcaggatcaggagcagat
cattttcgcatttccgccggttctgggctatggccttatgtaccaaaatctgtccagccgcttgccgtcatacaagctatgcgcctttgattttattgag
gaggaagaccggcttgaccgctatgcggatttgatccagaagctgcagccggaagggcctttaacattgtttggatattcagcgggatgcagcc
tggcgtttgaagctgcgaaaaagcttgaggaacaaggccgtattgttcagcggatcatcatggtggattcctataaaaaacaaggtgtcagtgatc
tggacggacgcacggttgaaagtgatgtcgaagcgttgatgaatgtcaatcgggacaatgaagcgctcaacagcgaagccgtcaaacacggc
ctcaagcaaaaaacacatgccttttactcatactacgtcaacctgatcagcacaggccaggtgaaagcagatattgatctgttgacttccggcgct
gattttgacatgccggaatggcttgcatcatgggaagaagctacaacaggtgtttaccgtgtgaaaagaggcttcggaacacacgcagaaatgc
tgcagggcgaaacgctagataggaatgcggagattttgctcgaatttcttaatacacaaaccgtaacggtttcataaatgaagtgatgaaaggag
gagacagccaatgagccaactcttcaaatcatttgatgcgtcggaaaaaacacagctcatctgttttccgtttgccggcggctattcggcgtcgttt
cgccctctccatgcttttttgcagggggagtgcgagatgctcgctgccgagccgccgggacacggcacgaatcaaacgtcagccattgaggat
ctcgaagagctgacggatttgtacaagcaagaactgaaccttcgccctgatcggccgtttgtgctgttcggacacagtatgggcggaatgatcac
cttcaggctggcgcaaaagcttgagcgtgaaggcatctttccgcaggcggttatcatttctgcaatccagccgcctcatattcagcggaagaaagt
gtcccacctgcctgatgatcagtttctcgatcatattatccaattaggcggaatgcccgcagagcttgttgaaaataaggaggtcatgtcctttttcct
gccttctttccgatcagattaccgggctcttgaacaatttgagctttacgatctggcccagatccagtcgcctgttcatgtctttaacgggcttgatgat
aaaaaatgcatacgagatgcggaagggtggaagaagtgggcaaaagacatcacattccatcaatttgacggcgggcacatgttcctgctgtca
caaacggaagaagtcgcagaacggatttttgcgatcttgaatcagcatccgatcattcaaccgtgaagccgccccgcagggcgctccgcaggc
cgcttccggaccactccggaagcggccgtgcggtcggaggatcc
ME-B0016

SEQ ID NO: 18

gaattcgcatgcatcgacaaaaatgtcatgaaagaatcgttgtaagacgctcttcgcaagggtgtcttttmgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaaagaaaaagtcatttctaaa
tggaatgagacggcaaaatccgagaagctggtcagccttcaggacatgttcgaaaagcaggcagttcttacgccagaacgtatcgctctcatgt
gtgatgacattcaagtcaactatcgaaagctcaatgaagaggcaaaccgcctcgcgcgtctgttgatcgaaaaagggattggtccggagcaattt
gtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggactcctcaggaagagatattgtgtgacttgtttgcagaggttctcggcagaaagcaaattggcattcatgacgatttc
tttgcgctcggagggcattccttgaaggccatgaccgccgcgtcccgcatcaagaaagagctcgggattgatcttccagtgaagcttttgtttgaa
gcgccgacgatcgccggcatttcagcgtatttgaaaaacgggggctctgatggcttgcaggatgtaacgataatgaatcaggatcaggagcag
atcattttcgcatttccgccggttctgggctatggccttatgtaccaaaatctgtccagccgcttgccgtcatacaagctatgcgcctttgattttattga
ggaggaagaccggcttgaccgctatgcggatttgatccagaagctgcagccggaagggcctttaacattgtttggatattcagcgggatgcagc
ctggcgtttgaagctgcgaaaaagcttgaggaacaaggccgtattgttcagcggatcatcatggtggattcctataaaaaacaaggtgtcagtgat
ctggacggacgcacggttgaaagtgatgtcgaagcgttgatgaatgtcaatcgggacaatgaagcgctcaacagcgaagccgtcaaacacgg
cctcaagcaaaaaacacatgccttttactcatactacgtcaacctgatcagcacaggccaggtgaaagcagatattgatctgttgacttccggcgc
tgattttgacatgccggaatggcttgcatcatgggaagaagctacaacaggtgtttaccgtgtgaaaagaggcttcggaacacacgcagaaatg
ctgcagggcgaaacgctagataggaatgcggagattttgctcgaatttcttaatacacaaaccgtaacggtttcataaatgaagtgatgaaaggag
gagacagccaatgagccaactcttcaaatcatttgatgcgtcggaaaaaacacagctcatctgttttccgtttgccggcggctattcggcgtcgttt
cgccctctccatgcttttttgcagggggagtgcgagatgctcgctgccgagccgccgggacacggcacgaatcaaacgtcagccattgaggat
ctcgaagagctgacggatttgtacaagcaagaactgaaccttcgccctgatcggccgtttgtgctgttcggacacagtatgggcggaatgatcac
cttcaggctggcgcaaaagcttgagcgtgaaggcatctttccgcaggcggttatcatttctgcaatccagccgcctcatattcagcggaagaaagt
gtcccacctgcctgatgatcagtttctcgatcatattatccaattaggcggaatgcccgcagagcttgttgaaaataaggaggtcatgtcctttttcct
gccttctttccgatcagattaccgggctcttgaacaatttgagctttacgatctggcccagatccagtcgcctgttcatgtctttaacgggcttgatgat
aaaaaatgcatacgagatgcggaagggtggaagaagtgggcaaaagacatcacattccatcaatttgacggcgggcacatgttcctgctgtca
caaacggaagaagtcgcagaacggatttttgcgatcttgaatcagcatccgatcattcaaccgtgaagccgccccgcagggcgctccgcaggc
cgcttccggaccactccggaagcggccgtgcggtcggaggatcc
ME-B0017

SEQ ID NO: 19

gaattcgcatgcatcgacaaaaatgtcatgaaagaatcgttgtaagacgctcttcgcaagggtgtctttttttgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaaagaaaaagtcatttctaaa
tggaatgagacggcaaaatccgagaagctggtcagccttcaggacatgttcgaaaagcaggcagttcttacgccagaacgtatcgctctcatgt
gtgatgacattcaagtcaactatcgaaagctcaatgaagaggcaaaccgcctcgcgcgtctgttgatcgaaaaagggattggtccggagcaattt
gtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggactcctcaggaagaaacaatcgcacaaatatggtctgaggttctcggcagaaagcaaattggcattcatgacgatt
tctttgcgctcggagggcattccttgaaggccatgaccgccgcgtcccgcatcaagaaagagctcgggattgatcttccagtgaagcttttgtttg
aagcgccgacgatcgccggcatttcagcgtatttgaaaaacgggggctctgatggcttgcaggatgtaacgataatgaatcaggatcaggagc
agatcattttcgcatttccgccggttctgggctatggccttatgtaccaaaatctgtccagccgcttgccgtcatacaagctatgcgcctttgattttatt
gaggaggaagaccggcttgaccgctatgcggatttgatccagaagctgcagccggaagggcctttaacattgtttggatattcagcgggatgca
gcctggcgtttgaagctgcgaaaaagcttgaggaacaaggccgtattgttcagcggatcatcatggtggattcctataaaaaacaaggtgtcagt
gatctggacggacgcacggttgaaagtgatgtcgaagcgttgatgaatgtcaatcgggacaatgaagcgctcaacagcgaagccgtcaaaca
cggcctcaagcaaaaaacacatgccttttactcatactacgtcaacctgatcagcacaggccaggtgaaagcagatattgatctgttgacttccgg
cgctgattttgacatgccggaatggcttgcatcatgggaagaagctacaacaggtgtttaccgtgtgaaaagaggcttcggaacacacgcagaa
atgctgcagggcgaaacgctagataggaatgcggagattttgctcgaatttcttaatacacaaaccgtaacggtttcataaatgaagtgatgaaag
gaggagacagccaatgagccaactcttcaaatcatttgatgcgtcggaaaaaacacagctcatctgttttccgtttgccggcggctattcggcgtc
gtttcgccctctccatgcttttttgcagggggagtgcgagatgctcgctgccgagccgccgggacacggcacgaatcaaacgtcagccattgag
gatctcgaagagctgacggatttgtacaagcaagaactgaaccttcgccctgatcggccgtttgtgctgttcggacacagtatgggcggaatgat
caccttcaggctggcgcaaaagcttgagcgtgaaggcatctttccgcaggcggttatcatttctgcaatccagccgcctcatattcagcggaaga
aagtgtcccacctgcctgatgatcagtttctcgatcatattatccaattaggcggaatgcccgcagagcttgttgaaaataaggaggtcatgtcctttt
tcctgccttctttccgatcagattaccgggctcttgaacaatttgagctttacgatctggcccagatccagtcgcctgttcatgtctttaacgggcttga
tgataaaaaatgcatacgagatgcggaagggtggaagaagtgggcaaaagacatcacattccatcaatttgacggcgggcacatgttcctgctg
tcacaaacggaagaagtcgcagaacggatttttgcgatcttgaatcagcatccgatcattcaaccgtgaagccgccccgcagggcgctccgca
ggccgcttccggaccactccggaagcggccgtgcggtcggaggatcc
ME-B0018

SEQ ID NO: 20

gaattcgcatgcatcgacaaaaatgtcatgaaagaatcgttgtaagacgctcttcgcaagggtgtctttttttgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaaagaaaaagtcatttctaaa
tggaatgagacggcaaaatccgagaagctggtcagccttcaggacatgttcgaaaagcaggcagttcttacgccagaacgtatcgctctcatgt
gtgatgacattcaagtcaactatcgaaagctcaatgaagaggcaaaccgcctcgcgcgtctgttgatcgaaaaagggattggtccggagcaattt
gtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggaacgagatggaagaaacaatcgcacaaatatggtctgaggttctcggcagaaagcaaattggcattcatgacgat
ttctttgcgctcggagggcattccttgaaggccatgaccgccgcgtcccgcatcaagaaagagctcgggattgatcttccagtgaagcttttgtttg
aagcgccgacgatcgccggcatttcagcgtatttgaaaaacgggggctctgatggcttgcaggatgtaacgataatgaatcaggatcaggagc
agatcattttcgcatttccgccggttctgggctatggccttatgtaccaaaatctgtccagccgcttgccgtcatacaagctatgcgcctttgattttatt
gaggaggaagaccggcttgaccgctatgcggatttgatccagaagctgcagccggaagggcctttaacattgtttggatattcagcgggatgca
gcctggcgtttgaagctgcgaaaaagcttgaggaacaaggccgtattgttcagcggatcatcatggtggattcctataaaaaacaaggtgtcagt
gatctggacggacgcacggttgaaagtgatgtcgaagcgttgatgaatgtcaatcgggacaatgaagcgctcaacagcgaagccgtcaaaca
cggcctcaagcaaaaaacacatgccttttactcatactacgtcaacctgatcagcacaggccaggtgaaagcagatattgatctgttgacttccgg
cgctgattttgacatgccggaatggcttgcatcatgggaagaagctacaacaggtgtttaccgtgtgaaaagaggcttcggaacacacgcagaa
atgctgcagggcgaaacgctagataggaatgcggagattttgctcgaatttcttaatacacaaaccgtaacggtttcataaatgaagtgatgaaag
gaggagacagccaatgagccaactcttcaaatcatttgatgcgtcggaaaaaacacagctcatctgttttccgtttgccggcggctattcggcgtc
gtttcgccctctccatgcttttttgcagggggagtgcgagatgctcgctgccgagccgccgggacacggcacgaatcaaacgtcagccattgag
gatctcgaagagctgacggatttgtacaagcaagaactgaaccttcgccctgatcggccgtttgtgctgttcggacacagtatgggcggaatgat
caccttcaggctggcgcaaaagcttgagcgtgaaggcatctttccgcaggcggttatcatttctgcaatccagccgcctcatattcagcggaaga
aagtgtcccacctgcctgatgatcagtttctcgatcatattatccaattaggcggaatgcccgcagagcttgttgaaaataaggaggtcatgtcctttt
tcctgccttctttccgatcagattaccgggctcttgaacaatttgagctttacgatctggcccagatccagtcgcctgttcatgtctttaacgggcttga
tgataaaaaatgcatacgagatgcggaagggtggaagaagtgggcaaaagacatcacattccatcaatttgacggcgggcacatgttcctgctg
tcacaaacggaagaagtcgcagaacggatttttgcgatcttgaatcagcatccgatcattcaaccgtgaagccgccccgcagggcgctccgca
ggccgcttccggaccactccggaagcggccgtgcggtcggaggatcc
ME-B0019

SEQ ID NO: 21

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaaagaaaaagtcatttctaaatggaatgagacggcaaaatccgagaagctggtc
agccttcaggacatgttcgaaaagcaggcagttcttacgccagaacgtatcgctctcatgtgtgatgacattcaagtcaactatcgaaagctc
aatgaagaggcaaaccgcctcgcgcgtctgttgatcgaaaaagggattggtccggagcaatttgtcgctttggcgctgccgcgttcccctga
gatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattggcacaaatatggtctgaggttctcggcagaaagcaaatt
ggcattcatgacgatttctttgcgctcggagggcattccttgaaggccatgaccgccgcgtcccgcatcaagaaagagctcgggattgatctt
ccagtgaagcttttgtttgaagcgccgacgatcgccggcatttcagcgtatttgaaaaacgggggctctgatggcttgcaggatgtaacgata
atgaatcaggatcaggagcagatcattttcgcatttccgccggttctgggctatggccttatgtaccaaaatctgtccagccgcttgccgtcata
caagctatgcgcctttgattttattgaggaggaagaccggcttgaccgctatgcggatttgatccagaagctgcagccggaagggcctttaac
attgtttggatattcagcgggatgcagcctggcgtttgaagctgcgaaaaagcttgaggaacaaggccgtattgttcagcggatcatcatggt
ggattcctataaaaaacaaggtgtcagtgatctggacggacgcacggttgaaagtgatgtcgaagcgttgatgaatgtcaatcgggacaatg
aagcgctcaacagcgaagccgtcaaacacggcctcaagcaaaaaacacatgccttttactcatactacgtcaacctgatcagcacaggcca
ggtgaaagcagatattgatctgttgacttccggcgctgattttgacatgccggaatggcttgcatcatgggaagaagctacaacaggtgtttac
cgtgtgaaaagaggcttcggaacacacgcagaaatgctgcagggcgaaacgctagatgggaatgcggagattttgctcgaatttcttaatac
acaaaccgtaacggtttcataaatgaagtgatgaaaggaggagacagccaatgagccaactcttcaaatcatttgatgcgtcggaaaaaaca
cagctcatctgttttccgtttgccggcggctattcggcgtcgtttcgccctctccatgcttttttgcagggggagtgcgagatgctcgctgccga
gccgccgggacacggcacgaatcaaacgtcagccattgaggatctcgaagagctgacggatttgtacaagcaagaactgaaccttcgccc
tgatcggccgtttgtgctgttcggacacagtatgggcggaatgatcaccttcaggctggcgcaaaagcttgagcgtgaaggcatctttccgc
aggcggttatcatttctgcaatccagccgcctcatattcagcggaagaaagtgtcccacctgcctgatgatcagtttctcgatcatattatccaat
taggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0020

SEQ ID NO: 22

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgctgttgccagaggagaaagaaaaagtcatttctaaatggaatgagacggcaaaatccgagaagctggt
cagccttcaggacatgttcgaaaagcaggcagttcttacgccagaacgtatcgctctcatgtgtgatgacattcaagtcaactatcgaaagct
caatgaagaggcaaaccgcctcgcgcgtctgttgatcgaaaaagggattggtccggagcaatttgtcgctttggcgctgccgcgttcccctg
agatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattgtgtgacttgtttgcagaggttctcggtttggcacgcgtcgg
tattcatgacgatttctttgcgctcggagggcattccttgaaggccatgaccgccgcgtcccgcatcaagaaagagctcgggattgatcttcc
agtgaagcttttgtttgaagcgccgacgatcgccggcatttcagcgtatttgaaaaacgggggctctgatggcttgcaggatgtaacgataat
gaatcaggatcaggagcagatcattttcgcatttccgccggttctgggctatggccttatgtaccaaaatctgtccagccgcttgccgtcatac
aagctatgcgcctttgattttattgaggaggaagaccggcttgaccgctatgcggatttgatccagaagctgcagccggaagggcctttaaca
ttgtttggatattcagcgggatgcagcctggcgtttgaagctgcgaaaaagcttgaggaacaaggccgtattgttcagcggatcatcatggtg
gattcctataaaaaacaaggtgtcagtgatctggacggacgcacggttgaaagtgatgtcgaagcgttgatgaatgtcaatcgggacaatga
agcgctcaacagcgaagccgtcaaacacggcctcaagcaaaaaacacatgccttttactcatactacgtcaacctgatcagcacaggccag
gtgaaagcagatattgatctgttgacttccggcgctgattttgacatgccggaatggcttgcatcatgggaagaagctacaacaggtgtttacc
gtgtgaaaagaggcttcggaacacacgcagaaatgctgcagggcgaaacgctagataggaatgcggagattttgctcgaatttcttaataca
caaaccgtaacggtttcataaatgaagtgatgaaaggaggagacagccaatgagccaactcttcaaatcatttgatgcgtcggaaaaaacac
agctcatctgttttccgtttgccggcggctattcggcgtcgtttcgccctctccatgcttttttgcagggggagtgcgagatgctcgctgccgag
ccgccgggacacggcacgaatcaaacgtcagccattgaggatctcgaagagctgacggatttgtacaagcaagaactgaaccttcgccct
gatcggccgtttgtgctgttcggacacagtatgggcggaatgatcaccttcaggctggcgcaaaagcttgagcgtgaaggcatctttccgca
ggcggttatcatttctgcaatccagccgcctcatattcagcggaagaaagtgtcccacctgcctgatgatcagtttctcgatcatattatccaatt
aggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0021

SEQ ID NO: 23

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaaagaaaaagtcatttctaaatggaatgagacggcaaaatccgagaagctggtc
agccttcaggacatgttcgaaaagcaggcagttcttacgccagaacgtatcgctctcatgtgtgatgacattcaagtcaactatcgaaagctc
aatgaagaggcaaaccgcctcgcgcgtctgttgatcgaaaaagggattggtccggagcaatttgtcgctttggcgctgccgcgttcccctga
gatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact.
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattgtgtgacttgtttgcagaggttctcggtttggcacgcgtcgg
tattcatgacgatttctttgcgctcggagggcattccttgaaggccatgaccgccgcgtcccgcatcaagaaagagctcgggattgatcttcc
agtgaagcttttgtttgaagcgccgacgatcgccggcatttcagcgtatttgaaaaacgggggctctgatggcttgcaggatgtaacgataat
gaatcaggatcaggagcagatcattttcgcatttccgccggttctgggctatggccttatgtaccaaaatctgtccagccgcttgccgtcatac
aagctatgcgcctttgattttattgaggaggaagaccggcttgaccgctatgcggatttgatccagaagctgcagccggaagggcctttaaca
ttgtttggatattcagcgggatgcagcctggcgtttgaagctgcgaaaaagcttgaggaacaaggccgtattgttcagcggatcatcatggtg
gattcctataaaaaacaaggtgtcagtgatctggacggacgcacggttgaaagtgatgtcgaagcgttgatgaatgtcaatcgggacaatga
agcgctcaacagcgaagccgtcaaacacggcctcaagcaaaaaacacatgccttttactcatactacgtcaacctgatcagcacaggccag
gtgaaagcagatattgatctgttgacttccggcgctgattttgacatgccggaatggcttgcatcatgggaagaagctacaacaggtgtttacc
gtgtgaaaagaggcttcggaacacacgcagaaatgctgcagggcgaaacgctagataggaatgcggagattttgctcgaatttcttaataca
caaaccgtaacggtttcataaatgaagtgatgaaaggaggagacagccaatgagccaactcttcaaatcatttgatgcgtcggaaaaaacac
agctcatctgttttccgtttgccggcggctattcggcgtcgtttcgccctctccatgcttttttgcagggggagtgcgagatgctcgctgccgag
ccgccgggacacggcacgaatcaaacgtcagccattgaggatctcgaagagctgacggatttgtacaagcaagaactgaaccttcgccct
gatcggccgtttgtgctgttcggacacagtatgggcggaatgatcaccttcaggctggcgcaaaagcttgagcgtgaaggcatctttccgca
ggcggttatcatttctgcaatccagccgcctcatattcagcggaagaaagtgtcccacctgcctgatgatcagtttctcgatcatattatccaatt
aggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0021

SEQ ID NO: 24

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaaagaaaaagtcatttctaaatggaatgagacggcaaaatccgagaagctggtc
agccttcaggacatgttcgaaaagcaggcagttcttacgccagaacgtatcgctctcatgtgtgatgacattcaagtcaactatcgaaagctc
aatgaagaggcaaaccgcctcgcgcgtctgttgatcgaaaaagggattggtccggagcaatttgtcgctttggcgctgccgcgttcccctga
gatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattggcacaaatatggtctgaggttctcggcagaaagcaaatt
ggcattcatgacgatttctttgcgctcggagggcattccttgaaggccatgaccgccgcgtcccgcatcaagaaagagctcgggattgatctt
ccagtgaagcttttgtttgaagcgccgacgatcgccggcatttcagcgtatttgaaaaacgggggctctgatggcttgcaggatgtaacgata
atgaatcaggatcaggagcagatcattttcgcatttccgccggttctgggctatggccttatgtaccaaaatctgtccagccgcttgccgtcata
caagctatgcgcctttgattttattgaggaggaagaccggcttgaccgctatgcggatttgatccagaagctgcagccggaagggcctttaac
attgtttggatattcagcgggatgcagcctggcgtttgaagctgcgaaaaagcttgaggaacaaggccgtattgttcagcggatcatcatggt
ggattcctataaaaaacaaggtgtcagtgatctggacggacgcacggttgaaagtgatgtcgaagcgttgatgaatgtcaatcgggacaatg
aagcgctcaacagcgaagccgtcaaacacggcctcaagcaaaaaacacatgccttttactcatactacgtcaacctgatcagcacaggcca
ggtgaaagcagatattgatctgttgacttccggcgctgattttgacatgccggaatggcttgcatcatgggaagaagctacaacaggtgtttac
cgtgtgaaaagaggcttcggaacacacgcagaaatgctgcagggcgaaacgctagataggaatgcggagattttgctcgaatttcttaatac
acaaaccgtaacggtttcataaatgaagtgatgaaaggaggagacagccaatgagccaactcttcaaatcatttgatgcgtcggaaaaaaca
cagctcatctgttttccgtttgccggcggctattcggcgtcgtttcgccctctccatgcttttttgcagggggagtgcgagatgctcgctgccga
gccgccgggacacggcacgaatcaaacgtcagccattgaggatctcgaagagctgacggatttgtacaagcaagaactgaaccttcgccc
tgatcggccgtttgtgctgttcggacacagtatgggcggaatgatcaccttcaggctggcgcaaaagcttgagcgtgaaggcatctttccgc
aggcggttatcatttctgcaatccagccgcctcatattcagcggaagaaagtgtcccacctgcctgatgatcagtttctcgatcatattatccaat
taggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0023

SEQ ID NO: 25

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgctgttgccagaggagaaagaaaaagtcatttctaaatggaatgagacggcaaaatccgagaagctggt
cagccttcaggacatgttcgaaaagcaggcagttcttacgccagaacgtatcgctctcatgtgtgatgacattcaagtcaactatcgaaagct
caatgaagaggcaaaccgcctcgcgcgtctgttgatcgaaaaagggattggtccggagcaatttgtcgctttggcgctgccgcgttcccctg
agatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattgtgtgacttgtttgcagaggttctcggtttggcacgcgtcgg
tattcatgacgatttctttgcgctcggagggcattccttgaaggccatgaccgccgcgtcccgcatcaagaaagagctcgggattgatcttcc
agtgaagcttttgtttgaagcgccgacgatcgccggcatttcagcgtatttgaaaaacgggggctctgatggcttgcaggatgtaacgataat
gaatcaggatcaggagcagatcattttcgcatttccgccggttctgggctatggccttatgtaccaaaatctgtccagccgcttgccgtcatac
aagctatgcgcctttgattttattgaggaggaagaccggcttgaccgctatgcggatttgatccagaagctgcagccggaagggcctttaaca
ttgtttggatattcagcgggatgcagcctggcgtttgaagctgcgaaaaagcttgaggaacaaggccgtattgttcagcggatcatcatggtg
gattcctataaaaaacaaggtgtcagtgatctggacggacgcacggttgaaagtgatgtcgaagcgttgatgaatgtcaatcgggacaatga
agcgctcaacagcgaagccgtcaaacacggcctcaagcaaaaaacacatgccttttactcatactacgtcaacctgatcagcacaggccag
gtgaaagcagatattgatctgttgacttccggcgctgattttgacatgccggaatggcttgcatcatgggaagaagctacaacaggtgtttacc
gtgtgaaaagaggcttcggaacacacgcagaaatgctgcagggcgaaacgctagataggaatgcggagattttgctcgaatttcttaataca
caaaccgtaacggtttcataaatgaagtgatgaaaggaggagacagccaatgagccaactcttcaaatcatttgatgcgtcggaaaaaacac
agctcatctgttttccgtttgccggcggctattcggcgtcgtttcgccctctccatgcttttttgcagggggagtgcgagatgctcgctgccgag
ccgccgggacacggcacgaatcaaacgtcagccattgaggatctcgaagagctgacggatttgtacaagcaagaactgaaccttcgccct
gatcggccgtttgtgctgttcggacacagtatgggcggaatgatcaccttcaggctggcgcaaaagcttgagcgtgaaggcatctttccgca
ggcggttatcatttctgcaatccagccgcctcatattcagcggaagaaagtgtcccacctgcctgatgatcagtttctcgatcatattatccaatt
aggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0024

SEQ ID NO: 26

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaaagaaaaagtcatttctaaatggaatgagacggcaaaatccgagaagctggtc
agccttcaggacatgttcgaaaagcaggcagttcttacgccagaacgtatcgctctcatgtgtgatgacattcaagtcaactatcgaaagctc
aatgaagaggcaaaccgcctcgcgcgtctgttgatcgaaaaagggattggtccggagcaatttgtcgctttggcgctgccgcgttcccctga
gatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattgtgtgacttgtttgcagaggttctcggtttggcacgcgtcgg
tattcatgacgatttctttgcgctcggagggcattccttgaaggccatgaccgccgcgtcccgcatcaagaaagagctcgggattgatcttcc
agtgaagcttttgtttgaagcgccgacgatcgccggcatttcagcgtatttgaaaaacgggggctctgatggcttgcaggatgtaacgataat
gaatcaggatcaggagcagatcattttcgcatttccgccggttctgggctatggccttatgtaccaaaatctgtccagccgcttgccgtcatac
aagctatgcgcctttgattttattgaggaggaagaccggcttgaccgctatgcggatttgatccagaagctgcagccggaagggcctttaaca
ttgtttggatattcagcgggatgcagcctggcgtttgaagctgcgaaaaagcttgaggaacaaggccgtattgttcagcggatcatcatggtg
gattcctataaaaaacaaggtgtcagtgatctggacggacgcacggttgaaagtgatgtcgaagcgttgatgaatgtcaatcgggacaatga
agcgctcaacagcgaagccgtcaaacacggcctcaagcaaaaaacacatgccttttactcatactacgtcaacctgatcagcacaggccag
gtgaaagcagatattgatctgttgacttccggcgctgattttgacatgccggaatggcttgcatcatgggaagaagctacaacaggtgtttacc
gtgtgaaaagaggcttcggaacacacgcagaaatgctgcagggcgaaacgctagataggaatgcggagattttgctcgaatttcttaataca
caaaccgtaacggtttcataaatgaagtgatgaaaggaggagacagccaatgagccaactcttcaaatcatttgatgcgtcggaaaaaacac
agctcatctgttttccgtttgccggcggctattcggcgtcgtttcgccctctccatgcttttttgcagggggagtgcgagatgctcgctgccgag
ccgccgggacacggcacgaatcaaacgtcagccattgaggatctcgaagagctgacggatttgtacaagcaagaactgaaccttcgccct
gatcggccgtttgtgctgttcggacacagtatgggcggaatgatcaccttcaggctggcgcaaaagcttgagcgtgaaggcatctttccgca
ggcggttatcatttctgcaatccagccgcctcatattcagcggaagaaagtgtcccacctgcctgatgatcagtttctcgatcatattatccaatt
aggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0025

SEQ ID NO: 27

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaagcagatgattctcaaaacatggaacgccacaggaaaaacgtatccatatata
acttttcatgagttgtttgagcagcaggcgaaaaagacgcctgatagagcggctgtcagctatgaaggtcaaacattgacgtatcgggagctt
gatgagaaaagcacacagctggccatttatttgcaggcgcatggagtgggtcctgaccgtctggcggggatctatgtggatcgatcgctgg
acatgctagtgggtttattagcgatcctcaaggctgggggagcgtatgtgccgctagacccgtcctatccggctgaacgattagaatacatgc
ttgaggacagtgaagttttcattacactgacgacatcggaattagtaaatacgttgagttggaacggtgtcacaacagcccttttagatcaagat
tgggatgaaattgctcaaacagcctctgatcgaaaagtgcttacacgcactgtcacgccagagaacttggcatatgtcatttatacatccggc
agcacaggaaagccaaaaggtgtcatgataccacataaagctttgacaaactttctcgtttcgatgggggaaacaccaggtcttacggcaga
ggataaaatgcttgctgtcacaacctactgttttgatattgcagctctggaattatttttgcctttaataaagggcgcacactgctatatttgtcaaa
cggagcatacaaaagacgttgaaaaactgaaacgggacatccgcgcgatcaaaccgacagtgatgcaggcaacccccgctacgtggaa
gatgctcttttattcaggctgggaaaatgaagagagcgtgaaaattttatgcggtggcgaagcattgcctgagacattaaaacgatatttcttag
atacgggcagcgaagcctggaatatgttcgggccaaccgaaacaactatctggtcagcggttcagcgcattaacgttgaatgctctcatgcc
acgataggaaggccaatcgccaatacacaaatctatattacggattctcagctcgcgccagtgccggcaggtgttccgggtgagctgtgcat
tgcaggagacggtgtggcgaagggctactacaaaaaggaagaattaacggattcgagattcattgacaacccttttgagcctgggtctaag
ctttatagaacgggagacatggcccgttggcttacgggagggcgaattgaatatataggccgcatcgataatcaagtaaaaatccgcggatt
ccgtattgaacttggtgatattgaaagcaggcttagtgagcatcccggcattctggaatgcgttgtggtcgcagatatggataacctagctgcc
tattatacagctaaacatgcaaatgcttctctcacagcgagagagctgcgtcattttgtgaaaaacgctttgcctgcctatatggtgccttcttatt
ttattcagcttgatcatatgccgttaactccgaacggaaagatagatagaaacagccttaagaatatcgatttatcaggggagcagctaaagc
aaaggcagacctctcctaagaacattcaggatactgtttttaccatttggcaggaagtgctgggcagaaagcaaattggcattcatgacgattt
ctttgcgctcggagggcattccttgaaggccatgaccgccgcgtcccgcatcaagaaagagctcgggattgatcttccagtgaagcttttgtt
tgaagcgccgacgatcgccggcatttcagcgtatttgaaaaacgggggctctgatggcttgcaggatgtaacgataatgaatcaggatcag
gagcagatcattttcgcatttccgccggttctgggctatggccttatgtaccaaaatctgtccagccgcttgccgtcatacaagctatgcgccttt
gattttattgaggaggaagaccggcttgaccgctatgcggatttgatccagaagctgcagccggaagggcctttaacattgtttggatattcag
cgggatgcagcctggcgtttgaagctgcgaaaaagcttgaggaacaaggccgtattgttcagcggatcatcatggtggattcctataaaaaa
caaggtgtcagtgatctggacggacgcacggttgaaagtgatgtcgaagcgttgatgaatgtcaatcgggacaatgaagcgctcaacagc
gaagccgtcaaacacggcctcaagcaaaaaacacatgccttttactcatactacgtcaacctgatcagcacaggccaggtgaaagcagata
ttgatctgttgacttccggcgctgattttgacatgccggaatggcttgcatcatgggaagaagctacaacaggtgtttaccgtgtgaaaagagg
cttcggaacacacgcagaaatgctgcagggcgaaacgctagataggaatgcggagattttgctcgaatttcttaatacacaaaccgtaacgg
tttcataaatgaagtgatgaaaggaggagacagccaatgagccaactcttcaaatcatttgatgcgtcggaaaaaacacagctcatctgttttc
cgtttgccggcggctattcggcgtcgtttcgccctctccatgcttttttgcagggggagtgcgagatgctcgctgccgagccgccgggacac
ggcacgaatcaaacgtcagccattgaggatctcgaagagctgacggatttgtacaagcaagaactgaaccttcgccctgatcggccgtttgt
gctgttcggacacagtatgggcggaatgatcaccttcaggctggcgcaaaagcttgagcgtgaaggcatctttccgcaggcggttatcattt
ctgcaatccagccgcctcatattcagcggaagaaagtgtcccacctgcctgatgatcagtttctcgatcatattatccaattaggcggaatgcc
cgcagagcttgttgaaaataaggaggtcatgtcctttttcctgccttctttccgatcagattaccgggctcttgaacaatttgagctttacgatctg
gcccagatccagtcgcctgttcatgtctttaacgggcttgatgataaaaaatgcatacgagatgcggaagggtggaagaagtgggcaaaag
acatcacattccatcaatttgacggcgggcacatgttcctgctgtcacaaacggaagaagtcgcagaacggatttttgcgatcttgaatcagc
atccgatcattcaaccgtgaagccgccccgcagggcgctccgcaggccgcttccggaccactccggaagcggccgtgcggtcggagga
tcc
ME-B0026

SEQ ID NO: 28

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaagcagatgattctcaaaacatggaacgccacaggaaaaacgtatccatatata
acttttcatgagttgtttgagcagcaggcgaaaaagacgcctgatagagcggctgtcagctatgaaggtcaaacattgacgtatcgggagctt
gatgagaaaagcacacagctggccatttatttgcaggcgcatggagtgggtcctgaccgtctggcggggatctatgtggatcgatcgctgg
acatgctagtgggtttattagcgatcctcaaggctgggggagcgtatgtgccgctagacccgtcctatccggctgaacgattagaatacatgc
ttgaggacagtgaagttttcattacactgacgacatcggaattagtaaatacgttgagttggaacggtgtcacaacagcccttttagatcaagat
tgggatgaaattgctcaaacagcctctgatcgaaaagtgcttacacgcactgtcacgccagagaacttggcatatgtcatttatacatccggc
agcacaggaaagccaaaaggtgtcatgataccacataaagctttgacaaactttctcgtttcgatgggggaaacaccaggtcttacggcaga
ggataaaatgcttgctgtcacaacctactgttttgatattgcagctctggaattatttttgcctttaataaagggcgcacactgctatatttgtcaaa
cggagcatacaaaagacgttgaaaaactgaaacgggacatccgcgcgatcaaaccgacagtgatgcaggcaacccccgctacgtggaa
gatgctcttttattcaggctgggaaaatgaagagagcgtgaaaattttatgcggtggcgaagcattgcctgagacattaaaacgatatttcttag
atacgggcagcgaagcctggaatatgttcgggccaaccgaaacaactatctggtcagcggttcagcgcattaacgttgaatgctctcatgcc
acgataggaaggccaatcgccaatacacaaatctatattacggattctcagctcgcgccagtgccggcaggtgttccgggtgagctgtgcat
tgcaggagacggtgtggcgaagggctactacaaaaaggaagaattaacggattcgagattcattgacaacccttttgagcctgggtctaag
ctttatagaacgggagacatggcccgttggcttacgggagggcgaattgaatatataggccgcatcgataatcaagtaaaaatccgcggatt
ccgtattgaacttggtgatattgaaagcaggcttagtgagcatcccggcattctggaatgcgttgtggtcgcagatatggataacctagctgcc
tattatacagctaaacatgcaaatgcttctctcacagcgagagagctgcgtcattttgtgaaaaacgctttgcctgcctatatggtgccttcttatt
ttattcagcttgatcatatgccgttaactccgaacggaaagatagatagaaacagccttaagaatatcgatttatcaggggagcagctaaagc
aaaggcagacctctcctaagaacattcaggatactgtttttaccatttggtctgaggttctcggcagaaagcaaattggcattcatgacgatttct
ttgcgctcggagggcattccttgaaggccatgaccgccgcgtcccgcatcaagaaagagctcgggattgatcttccagtgaagcttttgtttg
aagcgccgacgatcgccggcatttcagcgtatttgaaaaacgggggctctgatggcttgcaggatgtaacgataatgaatcaggatcagga
gcagatcattttcgcatttccgccggttctgggctatggccttatgtaccaaaatctgtccagccgcttgccgtcatacaagctatgcgcctttga
ttttattgaggaggaagaccggcttgaccgctatgcggatttgatccagaagctgcagccggaagggcctttaacattgtttggatattcagcg
ggatgcagcctggcgtttgaagctgcgaaaaagcttgaggaacaaggccgtattgttcagcggatcatcatggtggattcctataaaaaaca
aggtgtcagtgatctggacggacgcacggttgaaagtgatgtcgaagcgttgatgaatgtcaatcgggacaatgaagcgctcaacagcga
agccgtcaaacacggcctcaagcaaaaaacacatgccttttactcatactacgtcaacctgatcagcacaggccaggtgaaagcagatattg
atctgttgacttccggcgctgattttgacatgccggaatggcttgcatcatgggaagaagctacaacaggtgtttaccgtgtgaaaagaggctt
cggaacacacgcagaaatgctgcagggcgaaacgctagataggaatgcggagattttgctcgaatttcttaatacacaaaccgtaacggttt
cataaatgaagtgatgaaaggaggagacagccaatgagccaactcttcaaatcatttgatgcgtcggaaaaaacacagctcatctgttttccg
tttgccggcggctattcggcgtcgtttcgccctctccatgcttttttgcagggggagtgcgagatgctcgctgccgagccgccgggacacgg
cacgaatcaaacgtcagccattgaggatctcgaagagctgacggatttgtacaagcaagaactgaaccttcgccctgatcggccgtttgtgc
tgttcggacacagtatgggcggaatgatcaccttcaggctggcgcaaaagcttgagcgtgaaggcatctttccgcaggcggttatcatttctg
caatccagccgcctcatattcagcggaagaaagtgtcccacctgcctgatgatcagtttctcgatcatattatccaattaggcggaatgcccgc
agagcttgttgaaaataaggaggtcatgtcctttttcctgccttctttccgatcagattaccgggctcttgaacaatttgagctttacgatctggcc
cagatccagtcgcctgttcatgtctttaacgggcttgatgataaaaaatgcatacgagatgcggaagggtggaagaagtgggcaaaagaca
tcacattccatcaatttgacggcgggcacatgttcctgctgtcacaaacggaagaagtcgcagaacggatttttgcgatcttgaatcagcatcc
gatcattcaaccgtgaagccgccccgcagggcgctccgcaggccgcttccggaccactccggaagcggccgtgcggtcggaggatcc
ME-B0027

SEQ ID NO: 29

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaagcagatgattctcaaaacatggaacgccacaggaaaaacgtatccatatata
acttttcatgagttgtttgagcagcaggcgaaaaagacgcctgatagagcggctgtcagctatgaaggtcaaacattgacgtatcgggagctt
gatgagaaaagcacacagctggccatttatttgcaggcgcatggagtgggtcctgaccgtctggcggggatctatgtggatcgatcgctgg
acatgctagtgggtttattagcgatcctcaaggctgggggagcgtatgtgccgctagacccgtcctatccggctgaacgattagaatacatgc
ttgaggacagtgaagttttcattacactgacgacatcggaattagtaaatacgttgagttggaacggtgtcacaacagcccttttagatcaagat
tgggatgaaattgctcaaacagcctctgatcgaaaagtgcttacacgcactgtcacgccagagaacttggcatatgtcatttatacatccggc
agcacaggaaagccaaaaggtgtcatgataccacataaagctttgacaaactttctcgtttcgatgggggaaacaccaggtcttacggcaga
ggataaaatgcttgctgtcacaacctactgttttgatattgcagctctggaattatttttgcctttaataaagggcgcacactgctatatttgtcaaa
cggagcatacaaaagacgttgaaaaactgaaacgggacatccgcgcgatcaaaccgacagtgatgcaggcaacccccgctacgtggaa
gatgctcttttattcaggctgggaaaatgaagagagcgtgaaaattttatgcggtggcgaagcattgcctgagacattaaaacgatatttcttag
atacgggcagcgaagcctggaatatgttcgggccaaccgaaacaactatctggtcagcggttcagcgcattaacgttgaatgctctcatgcc
acgataggaaggccaatcgccaatacacaaatctatattacggattctcagctcgcgccagtgccggcaggtgttccgggtgagctgtgcat
tgcaggagacggtgtggcgaagggctactacaaaaaggaagaattaacggattcgagattcattgacaacccttttgagcctgggtctaag
ctttatagaacgggagacatggcccgttggcttacgggagggcgaattgaatatataggccgcatcgataatcaagtaaaaatccgcggatt
ccgtattgaacttggtgatattgaaagcaggcttagtgagcatcccggcattctggaatgcgttgtggtcgcagatatggataacctagctgcc
tattatacagctaaacatgcaaatgcttctctcacagcgagagagctgcgtcattttgtgaaaaacgctttgcctgcctatatggtgccttcttatt
ttattcagcttgatcatatgccgttaactccgaacggaaagatagatagaaacagccttaagaatatcgatttatcaggggagcagctaaagc
aaaggcagacctctcctaagaacattcaggatactgttgcacaaatatggtctgaggttctcggcagaaagcaaattggcattcatgacgattt
ctttgcgctcggagggcattccttgaaggccatgaccgccgcgtcccgcatcaagaaagagctcgggattgatcttccagtgaagcttttgtt
tgaagcgccgacgatcgccggcatttcagcgtatttgaaaaacgggggctctgatggcttgcaggatgtaacgataatgaatcaggatcag
gagcagatcattttcgcatttccgccggttctgggctatggccttatgtaccaaaatctgtccagccgcttgccgtcatacaagctatgcgccttt
gattttattgaggaggaagaccggcttgaccgctatgcggatttgatccagaagctgcagccggaagggcctttaacattgtttggatattcag
cgggatgcagcctggcgtttgaagctgcgaaaaagcttgaggaacaaggccgtattgttcagcggatcatcatggtggattcctataaaaaa
caaggtgtcagtgatctggacggacgcacggttgaaagtgatgtcgaagcgttgatgaatgtcaatcgggacaatgaagcgctcaacagc
gaagccgtcaaacacggcctcaagcaaaaaacacatgccttttactcatactacgtcaacctgatcagcacaggccaggtgaaagcagata
ttgatctgttgacttccggcgctgattttgacatgccggaatggcttgcatcatgggaagaagctacaacaggtgtttaccgtgtgaaaagagg
cttcggaacacacgcagaaatgctgcagggcgaaacgctagataggaatgcggagattttgctcgaatttcttaatacacaaaccgtaacgg
tttcataaatgaagtgatgaaaggaggagacagccaatgagccaactcttcaaatcatttgatgcgtcggaaaaaacacagctcatctgttttc
cgtttgccggcggctattcggcgtcgtttcgccctctccatgcttttttgcagggggagtgcgagatgctcgctgccgagccgccgggacac
ggcacgaatcaaacgtcagccattgaggatctcgaagagctgacggatttgtacaagcaagaactgaaccttcgccctgatcggccgtttgt
gctgttcggacacagtatgggcggaatgatcaccttcaggctggcgcaaaagcttgagcgtgaaggcatctttccgcaggcggttatcattt
ctgcaatccagccgcctcatattcagcggaagaaagtgtcccacctgcctgatgatcagtttctcgatcatattatccaattaggcggaatgcc
cgcagagcttgttgaaaataaggaggtcatgtcctttttcctgccttctttccgatcagattaccgggctcttgaacaatttgagctttacgatctg
gcccagatccagtcgcctgttcatgtctttaacgggcttgatgataaaaaatgcatacgagatgcggaagggtggaagaagtgggcaaaag
acatcacattccatcaatttgacggcgggcacatgttcctgctgtcacaaacggaagaagtcgcagaacggatttttgcgatcttgaatcagc
atccgatcattcaaccgtgaagccgccccgcagggcgctccgcaggccgcttccggaccactccggaagcggccgtgcggtcggagga
tcc
ME-B0028

SEQ ID NO: 30

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaaagaaaaagtcatttctaaatggaatgagacggcaaaatccgagaagctggtc
agccttcaggacatgttcgaaaagcaggcagttcttacgccagaacgtatcgctctcatgtgtgatgacattcaagtcaactatcgaaagctc
aatgaagaggcaaaccgcctcgcgcgtctgttgatcgaaaaagggattggtccggagcaatttgtcgctttggcgctgccgcgttcccctga
gatggtggcttcaatgcttggtgtgctcaagactggtgcggcgtatctcccccttgatccggagtttccagccgaccgcatttcttacatgctg
gaggatgcgaaaccttcatgcatcataacgactgaggaaatagcagccagtctgcctgatgacctggctgtacctgagcttgtgcttgatca
ggctgttacacaggagattataaaacgctattcgccggaaaatcaggatgtatcggtttcgcttgaccatcctgcgtatatcatctatacctcag
gatcaacaggaagaccgaagggagtcgttgtgacacagaaaagcttaagcaattttctgctgtctatgcaggaggcattttctctaggagaa
gaagacaggctgttggctgtgacgactgtcgcttttgatatttcagcattggagttatatcttccgctgattagcggagcgcaaatcgtgatcgc
aaagaaagaaacgatccgtgagccgcaggcattagctcaaatgattgaaaatttcgatattaacattatgcaggcgacaccgacactgtggc
acgctttggtaacgagtgaacctgagaaacttcgggggcttagagtgcttgtaggaggcgaggcgctgccgagcggtcttttgcaggaact
tcaagaccttcattgttcagtcacgaacttatacggtcccactgaaacaacgatttggtctgccgcagcttttcttgaagaagggctgaagggc
gttcctccgattgggaaaccgatttggaacacgcaggtgtatgtgcttgataatggattgcagccggtgccgccgggcgttgtcggagagct
ttatattgcaggaaccggcttggccagaggttatttccatcgtcctgatttaacggcggagcgcttcgttgcagatccatacggaccgccggg
aactcggatgtatcggaccggagaccaggcccgctggcgcgccgatgggtctttggattatatcgggcgggcggatcatcaaatcaaaatt
cgcggattccgaattgaacttggagaaattgatgccgtgcttgccaatcacccgcacattgaacaggccgcggttgtcgtacgggaagatca
gccgggagacaaacgattggcggcttatgtagtcgctgatgctgctattgatactgcagagcttcgtcgttatatgggggctagtcttcctgatt
atatggtaccgtcggcgtttgtggagatggacgagctgccgttaacacctaatggcaagcttgaccggaaagcactgccggcaccagactt
cagcacatctgtcagtgatcgggccccgcggactcctcaggaagagatattgtgtgacttgtttgcagaggttctcggtttggcacgcgtcgg
tattcatgacgatttctttgcgctcggagggcattccttgaaggccatgaccgccgcgtcccgcatcaagaaagagctcgggattgatcttcc
agtgaagcttttgtttgaagcgccgacgatcgccggcatttcagcgtatttgaaaaacgggggctctgatggcttgcaggatgtaacgataat
gaatcaggatcaggagcagatcattttcgcatttccgccggttctgggctatggccttatgtaccaaaatctgtccagccgcttgccgtcatac
aagctatgcgcctttgattttattgaggaggaagaccggcttgaccgctatgcggatttgatccagaagctgcagccggaagggcctttaaca
ttgtttggatattcagcgggatgcagcctggcgtttgaagctgcgaaaaagcttgaggaacaaggccgtattgttcagcggatcatcatggtg
gattcctataaaaaacaaggtgtcagtgatctggacggacgcacggttgaaagtgatgtcgaagcgttgatgaatgtcaatcgggacaatga
agcgctcaacagcgaagccgtcaaacacggcctcaagcaaaaaacacatgccttttactcatactacgtcaacctgatcagcacaggccag
gtgaaagcagatattgatctgttgacttccggcgctgattttgacatgccggaatggcttgcatcatgggaagaagctacaacaggtgtttacc
gtgtgaaaagaggcttcggaacacacgcagaaatgctgcagggcgaaacgctagataggaatgcggagattttgctcgaatttcttaataca
caaaccgtaacggtttcataaatgaagtgatgaaaggaggagacagccaatgagccaactcttcaaatcatttgatgcgtcggaaaaaacac
agctcatctgttttccgtttgccggcggctattcggcgtcgtttcgccctctccatgcttttttgcagggggagtgcgagatgctcgctgccgag
ccgccgggacacggcacgaatcaaacgtcagccattgaggatctcgaagagctgacggatttgtacaagcaagaactgaaccttcgccct
gatcggccgtttgtgctgttcggacacagtatgggcggaatgatcaccttcaggctggcgcaaaagcttgagcgtgaaggcatctttccgca
ggcggttatcatttctgcaatccagccgcctcatattcagcggaagaaagtgtcccacctgcctgatgatcagtttctcgatcatattatccaatt
aggcggaatgcccgcagagcttgttgaaaataaggaggtcatgtcctttttcctgccttctttccgatcagattaccgggctcttgaacaatttg
agctttacgatctggcccagatccagtcgcctgttcatgtctttaacgggcttgatgataaaaaatgcatacgagatgcggaagggtggaaga
agtgggcaaaagacatcacattccatcaatttgacggcgggcacatgttcctgctgtcacaaacggaagaagtcgcagaacggatttttgcg
atcttgaatcagcatccgatcattcaaccgtgaagccgccccgcagggcgctccgcaggccgcttccggaccactccggaagcggccgt
gcggtcggaggatcc
ME-B0029

SEQ ID NO: 31

gaattcgcatgcatcgacaaaaatgtcatgaaagaatcgttgtaagacgctcttcgcaagggtgtctttttttgcctttttttcggtttttgcgcggt
acacatagtcatgtaaagattgtaaattgcattcagcaataaaaaaagattgaacgcagcagtttggtttaaaaatttttatttttctgtaaataatgt
ttagtggaaatgattgcggcatcccgcaaaaaatattgctgtaaataaactggaatctttcggcatcccgcatgaaacttttcacccatttttcgg
tgataaaaacatttttttcatttaaactgaacggtagaaagataaaaaatattgaaaacaatgaataaatagccaaaattggtttcttattagggtg
gggtcttgcggtctttatccgcttatgttaaacgccgcaatgctgactgacggcagcctgctttaatagcggccatctgttttttgattggaagca
ctgctttttaagtgtagtactttgggctatttcggctgttagttcataagaattaaaagctgatatggataagaaagagaaaatgcgttgcacatgt
tcactgcttataaagattaggggaggtatgacaatatggaaataactttttaccctttaacggatgcacaaaaacgaatttggtacacagaaaa
attttatcctcacacgagcatttcaaatcttgcggggattggtaagctggtttcagctgatgcgattgattatgtgcttgttgagcaggcgattcaa
gagtttattcgcagaaatgacgccatgcgccttcggttgcggctagatgaaaacggggagcctgttcaatatattagcgagtatcggcctgtt
gatataaaacatactgacactactgaagatccgaatgcgatagagtttatttcacaatggagccgggaggaaacgaagaaacctttgccgct
atacgattgtgatttgttccgtttttccttgttcaccataaaggaaaatgaagtgtggttttacgcaaatgttcatcacgtgatttctgatggtatctc
catgaatattctcgggaatgcgatcatgcacatttatttagaattagccagcggctcagagacaaaagaaggaatctcgcattcatttatcgatc
atgttttatctgaacaggaatatgctcaatcgaagcggtttgaaaaggacaaggcgttttggaacaaacaatttgaatcggtgcctgaacttgtt
tccttgaaacggaatgcatccgcagggggaagtttagatgctgagaggttctctaaagatgtgcctgaagcgcttcatcagcagattctgtcg
ttttgtgaggcgaataaagtcagtgttctttcggtatttcaatcgctgctcgccgcctatttgtacagggtcagcggccagaatgatgttgtgacg
ggaacatttatgggcaaccggacaaatgcgaaagagaagcagatgcttggcatgtttgtttctacggttccgcttcggacaaacattgacgg
cgggcaggcgttttcagaatttgtcaaagaccggatgaaggatctgatgaagacacttcgccaccaaaagtatccgtataatctcctaatcaa
cgatttgcgtgaaacaaagagctctctgaccaagctgttcacggtttctcttgaatatcaagtgatgcagtggcagaaagaagaggatcttgc
ctttttgactgagccgattttcagcggcagcggattaaatgatgtctcaattcatgtaaaggatcgatgggatactgggaaactcaccatagatt
ttgattaccgcactgatttattttcacgtgaagaaatcaacatgatttgtgagcgcatgattaccatgctggagaacgcgttaacgcatccagaa
catacaattgatgaattaacactgatttctgatgcggagaagcagatgattctcaaaacatggaacgccacaggaaaaacgtatccatatata
acttttcatgagttgtttgagcagcaggcgaaaaagacgcctgatagagcggctgtcagctatgaaggtcaaacattgacgtatcgggagctt
gatgagaaaagcacacagctggccatttatttgcaggcgcatggagtgggtcctgaccgtctggcggggatctatgtggatcgatcgctgg
acatgctagtgggtttattagcgatcctcaaggctgggggagcgtatgtgccgctagacccgtcctatccggctgaacgattagaatacatgc
ttgaggacagtgaagttttcattacactgacgacatcggaattagtaaatacgttgagttggaacggtgtcacaacagcccttttagatcaagat
tgggatgaaattgctcaaacagcctctgatcgaaaagtgcttacacgcactgtcacgccagagaacttggcatatgtcatttatacatccggc
agcacaggaaagccaaaaggtgtcatgataccacataaagctttgacaaactttctcgtttcgatgggggaaacaccaggtcttacggcaga
ggataaaatgcttgctgtcacaacctactgttttgatattgcagctctggaattatttttgcctttaataaagggcgcacactgctatatttgtcaaa
cggagcatacaaaagacgttgaaaaactgaaacgggacatccgcgcgatcaaaccgacagtgatgcaggcaacccccgctacgtggaa
gatgctcttttattcaggctgggaaaatgaagagagcgtgaaaattttatgcggtggcgaagcattgcctgagacattaaaacgatatttcttag
atacgggcagcgaagcctggaatatgttcgggccaaccgaaacaactatctggtcagcggttcagcgcattaacgttgaatgctctcatgcc
acgataggaaggccaatcgccaatacacaaatctatattacggattctcagctcgcgccagtgccggcaggtgttccgggtgagctgtgcat
tgcaggagacggtgtggcgaagggctactacaaaaaggaagaattaacggattcgagattcattgacaacccttttgagcctgggtctaag
ctttatagaacgggagacatggcccgttggcttacgggagggcgaattgaatatataggccgcatcgataatcaagtaaaaatccgcggatt
ccgtattgaacttggtgatattgaaagcaggcttagtgagcatcccggcattctggaatgcgttgtggtcgcagatatggataacctagctgcc
tattatacagctaaacatgcaaatgcttctctcacagcgagagagctgcgtcattttgtgaaaaacgctttgcctgcctatatggtgccttcttatt
ttattcagcttgatcatatgccgttaactccgaacggaaagatagatagaaacagccttaagaatatcgatttatcaggggagcagctaaagc
aaaggcagacctctcctaagaacattcaggatactgttgcacaaatatggtctgaggttctcggcagaaagcaaattggcattcatgacgattt
ctttgcgctcggagggcattccttgaaggccatgaccgccgcgtcccgcatcaagaaagagctcgggattgatcttccagtgaagcttttgtt
tgaagcgccgacgatcgccggcatttcagcgtatttgaaaaacgggggctctgatggcttgcaggatgtaacgataatgaatcaggatcag
gagcagatcattttcgcatttccgccggttctgggctatggccttatgtaccaaaatctgtccagccgcttgccgtcatacaagctatgcgccttt
gattttattgaggaggaagaccggcttgaccgctatgcggatttgatccagaagctgcagccggaagggcctttaacattgtttggatattcag
cgggatgcagcctggcgtttgaagctgcgaaaaagcttgaggaacaaggccgtattgttcagcggatcatcatggtggattcctataaaaaa
caaggtgtcagtgatctggacggacgcacggttgaaagtgatgtcgaagcgttgatgaatgtcaatcgggacaatgaagcgctcaacagc
gaagccgtcaaacacggcctcaagcaaaaaacacatgccttttactcatactacgtcaacctgatcagcacaggccaggtgaaagcagata
ttgatctgttgacttccggcgctgattttgacatgccggaatggcttgcatcatgggaagaagctacaacaggtgtttaccgtgtgaaaagagg
cttcggaacacacgcagaaatgctgcagggcgaaacgctagataggaatgcggagattttgctcgaatttcttaatacacaaaccgtaacgg
tttcataaatgaagtgatgaaaggaggagacagccaatgagccaactcttcaaatcatttgatgcgtcggaaaaaacacagctcatctgttttc
cgtttgccggcggctattcggcgtcgtttcgccctctccatgcttttttgcagggggagtgcgagatgctcgctgccgagccgccgggacac
ggcacgaatcaaacgtcagccattgaggatctcgaagagctgacggatttgtacaagcaagaactgaaccttcgccctgatcggccgtttgt
gctgttcggacacagtatgggcggaatgatcaccttcaggctggcgcaaaagcttgagcgtgaaggcatctttccgcaggcggttatcattt
ctgcaatccagccgcctcatattcagcggaagaaagtgtcccacctgcctgatgatcagtttctcgatcatattatccaattaggcggaatgcc
cgcagagcttgttgaaaataaggaggtcatgtcctttttcctgccttctttccgatcagattaccgggctcttgaacaatttgagctttacgatctg
gcccagatccagtcgcctgttcatgtctttaacgggcttgatgataaaaaatgcatacgagatgcggaagggtggaagaagtgggcaaaag
acatcacattccatcaatttgacggcgggcacatgttcctgctgtcacaaacggaagaagtcgcagaacggatttttgcgatcttgaatcagc
atccgatcattcaaccgtgaagccgccccgcagggcgctccgcaggccgcttccggaccactccggaagcggccgtgcggtcggagga
tcc

SEQ ID NO: 32

atcgacaaaaatgtcatgaaagaatcgttgtaagacgctcttcgcaagggtgtctttttttgcctttttttcggtttttgcgcggtacacatagtcatgta
aagattgtaaattgcattcagcaataaaaaaagattgaacgcagcagtttggtttaaaaatttttatttttctgtaaataatgtttagtggaaatgattgcg
gcatcccgcaaaaaatattgctgtaaataaactggaatctttcggcatcccgcatgaaacttttcacccatttttcggtgataaaaacatttttttcattta
aactgaacggtagaaagataaaaaatattgaaaacaatgaataaatagccaaaattggtttcttattagggtggggtcttgcggtctttatccgcttat
gttaaacgccgcaatgctgactgacggcagcctgctttaatagcggccatctgttttttgattggaagcactgctttttaagtgtagtactttgggctat
ttcggctgttagttcataagaattaaaagctgatatggataagaaagagaaaatgcgttgcacatgttcactgcttataaagattaggggaggtatg
acaat

SEQ ID NO: 33

Atgagccaactcttcaaatcatttgatgcgtcggaaaaaacacagctcatctgttttccgtttgccggcggctattcggcgtcgtttcgccctctcca
tgcttttttgcagggggagtgcgagatgctcgctgccgagccgccgggacacggcacgaatcaaacgtcagccattgaggatctcgaagagct
gacggatttgtacaagcaagaactgaaccttcgccctgatcggccgtttgtgctgttcggacacagtatgggcggaatgatcaccttcaggctgg
cgcaaaagcttgagcgtgaaggcatctttccgcaggcggttatcatttctgcaatccagccgcctcatattcagcggaagaaagtgtcccacctg
cctgatgatcagtttctcgatcatattatccaattaggcggaatgcccgcagagcttgttgaaaataaggaggtcatgtcctttttcctgccttctttcc
gatcagattaccgggctcttgaacaatttgagctttacgatctggcccagatccagtcgcctgttcatgtctttaacgggcttgatgataaaaaatgc
atacgagatgcggaagggtggaagaagtgggcaaaagacatcacattccatcaatttgacggcgggcacatgttcctgctgtcacaaacggaa
gaagtcgcagaacggatttttgcgatcttgaatcagcatccgatcattcaaccgtga

SEQ ID NO: 34

CACGAGCATTTCAAATCTTG

SEQ ID NO: 35

CTGAACAATACGGCCTTG

SEQ ID NO: 36

CACGAGCATTTCAAATCTTG

SEQ ID NO: 37

CTGAACAATACGGCCTTG

SEQ ID NO: 38

CACGAGCATTTCAAATCTTG

SEQ ID NO: 39

CTGAACAATACGGCCTTG

SEQ ID NO: 40

DILQLGMIWK

SEQ ID NO: 41

DILQLGLIWK

SEQ ID NO: 42

DILQLGVIWK
SrfAA

SEQ ID NO: 43

MEITFYPLTDAQKRIWYTEKFYPHTSISNLAGIGKLVSADAIDYVLVEQAIQEFIRRNDAMR
LRLRLDENGEPVQYISEYRPVDIKHTDTTEDPNAIEFISQWSREETKKPLPLYDCDLFRFSLF
TIKENEVWFYANVHHVISDGISMNILGNAIMHIYLELASGSETKEGISHSFIDHVLSEQEYA
QSKRFEKDKAFWNKQFESVPELVSLKRNASAGGSLDAERFSKDVPEALHQQILSFCEANK
VSVLSVFQSLLAAYLYRVSGQNDVVTGTFMGNRTNAKEKQMLGMFVSTVPLRTNIDGGQ
AFSEFVKDRMKDLMKTLRHQKYPYNLLINDLRETKSSLTKLFTVSLEYQVMQWQKEEDL
AFLTEPIFSGSGLNDVSIHVKDRWDTGKLTIDFDYRTDLFSREEINMICERMITMLENALTH
PEHTIDELTLISDAEKEKLLARAGGKSVSYRKDMTIPELFQEKAELLSDHPAVVFEDRTLSY
RTLHEQSARIANVLKQKGVGPDSPVAVLIERSERMITAIMGILKAGGAYVPIDPGFPAERIQ
YILEDCGADFILTESKVAAPEADAELIDLDQAIEEGAEESLNADVNARNLAYIIYTSGTTGR
PKGVMIEHRQVHHLVESLQQTIYQSGSQTLRMALLAPFHFDASVKQIFASLLLGQTLYIVP
KKTVTNGAALTAYYRKNSIEATDGTPAHLQMLAAAGDFEGLKLKHMLIGGEGLSSVVAD
KLLKLFKEAGTAPRLTNVYGPTETCVDASVHPVIPENAVQSAYVPIGKALGNNRLYILDQ
KGRLQPEGVAGELYIAGDGVGRGYLHLPELTEEKFLQDPFVPGDRMYRTGDVVRWLPDG
TIEYLGREDDQVKVRGYRIELGEIEAVIQQAPDVAKAVVLARPDEQGNLEVCAYVVQKPG
SEFAPAGLREHAARQLPDYMVPAYFTEVTEIPLTPSGKVDRRKLFALEVKAVSGTAYTAP
RNETEKAIAAIWQDVLNVEKAGIFDNFFETGGHSLKAMTLLTKIHKETGIEIPLQFLFEHPTI
TALAEEADHRESKAFAVIEPAEKQEHYPLSLAQQRTYIVSQFEDAGVGYNMPAAAILEGP
LDIQKLERAFQGLIRRHESLRTSFVLENSTPRQKIHDSVDFNIEMIERGGRSDEAIMASFVRT
FDLAKAPLFRIGLLGLEENRHMLLFDMHHLISDGVSIGIMLEELARIYKGEQLPDLRLQYK
DYAVWQSRQAAEGYKKDQAYWKEVFAGELPVLQLLSDYPRPPVQSFEGDRVSIKLDAG
VKDRLNRLAEQNGATLYMVMLSAYYTLLSKYTGQDDIIVGTPSAGRNHSDTEGIIGMFVN
TLAIRSEVKQNETFTQLISRVRKRVLDAFSHQDYPFEWLVEDLNIPRDVSRHPLFDTMFSL
QNATEGIPAVGDLSLSVQETNFKIAKFDLTVQARETDEGIEIDVDYSTKLFKQSTADRLLT
HFARLLEDAAADPEKPISEYKLLSEEEAASQIQQFNPGRTPYPKDKTIVQLFEEQAANTPDH
TALQYEGESLTYRELNERANRLARGILSLGAGEGRTAAVLCERSMDMIVSILAVLKSGSA
YVPIDPEHPIQRMQHFFRDSGAKVLLTQRKLKALAEEAEFKGVIVLADEEESYHADARNL
ALPLDSAAMANLTYTSGTTGTPKGNIVTHANILRTVKETNYLSITEQDTILGLSNYVFDAF
MFDMFGSLLNGAKLVLIPKETVLDMARLSRVIERENISILMITTALFHLLVDLNPACLSTLR
KIMFGGERASVEHVRKALQTVGKGKLLHMYGPSESTVFATYHPVDELEEHTLSVPIGKPV
SNTEVYILDRTGHVQPAGIAGELCVSGEGLVKGYYNRPELTEEKFVPHPFTSGERMYKTG
DLARWLPNGDIEFIGRIDHQVKIRGQRIELGEIEHQLQTHDRVQESVVLAVDQGAGDKLLC
AYYVGEGDISSQEMREHAAKDLPAYMVPAVFIQMDELPLTGNGKIDRRALPIPDANVSRG
VSYVAPRNGTEQKVADIWAQVLQAEQVGAYDHFFDIGGHSLAGMKMLALVHQELGVEL
SLKDLFQSPTVEGLAQVIASAEKGTAASISPAEKQDTYPVSSPQKRMYVLQQLEDAQTSY
NMPAVLRLTGELDVERLNSVMQQLMQRHEALRTTFEIKDGETVQRIWEEAECEIAYFEAP
EEETERIVSEFIKPFKIDQLPLFRIGLIKHSDTEHVLLFDMHHIISDGASVGVLIEELSKLYDG
ETLEPLRIQYKDYAVWQQQFIQSELYKKQEEHWLKELDGELPVLTLPTDYSRPAVQTFEG
DRIAFSLEAGKADALRRLAKETDSTLYMVLLASYSAFLSKISGQDDIIVGSPVAGRSQADV
SRVIGMFVNTLALRTYPKGEKTFADYLNEVKETALSAFDAQDYPLEDLIGNVQVQRDTSR
NPLFDAVFSMQNANIKDLTMKGIQLEPHPFERKTAKFDLTLTADETDGGLTFVLEYNTAL
FKQETIERWKQYWMELLDAVTGNPNQPLSSLSLVTETEKQALLEAWKGKALPVPTDKTV
HQLFEETAQRHKDRPAVTYNGQSWTYGELNAKANRLARILMDCGISPDDRVGVLTKPSL
EMSAAVLGVLKAGAAFVPIDPDYPDQRIEYILQDSGAKLLLKQEGISVPDSYTGDVILLDG
SRTILSLPLDENDEENPETAVTAENLAYMIYTSGTTGQPKGVMVEHHALVNLCFWHHDAF
SMTAEDRSAKYAGFGFDASIWEMFPTWTIGAELHVIEEAIRLDIVRLNDYFETNGVTITFLP
TQLAEQFMELENTSLRVLLTGGDKLKRAVKKPYTLVNNYGPTENTVVATSAEIHPEEGSL
SIGRAIANTRVYILGEGNQVQPEGVAGELCVAGRGLARGYLNREDETAKRFVADPFVPGE
RMYRTGDLVKWTGGGIEYIGRIDQQVKVRGYRIELSEIEVQLAQLSEVQDAAVTAVKDK
GGNTAIAAYVTPESADIEALKSALKETLPDYMIPAFWVTLNELPVTANGKVDRKALPEPDI
EAGSGEYKAPTTDMEELLAGIWQDVLGMSEVGVTDNFFSLGGDSIKGIQMASRLNQHGW
KLEMKDLFQHPTIEELTQYVERAEGKQADQGPVEGEVILTPIQRWFFEKNFTNKHHWNQS
VMLHAKKGFDPERVEKTLQALIEHHDALRMVYREGQEDVIQYNRGLEAASAQLEVIQIEG
QAADYEDRIEREAERLQSSIDLQEGGLLKAGLFQAEDGDHLLLAIHHLVVDGVSWRILLE
DFAAVYTQLEQGNEPVLPQKTHSFAEYAERLQDFANSKAFLKEKEYWRQLEEQAVAAKL
PKDRESGDQRMKHTKTIEFSLTAEETEQLTTKVHEAYHTEMNDILLTAFGLAMKEWTGQ
DRVSVHLEGHGREEIIEDLTISRTVGWFTSMYPMVLDMKHADDLGYQLKQMKEDIRHVP
NKGVGYGILRYLTAPEHKEDVAFSIQPDVSFNYLGQFDEMSDAGLFTRSELPSGQSLSPET
EKPNALDVVGYIENGKLTMSLAYHSLEFHEKTVQTFSDSFKAHLLRIIEHCLSQDGTELTPS
DLGDDDLTLDELDKLMEIF
SrfAB

SEQ ID NO: 44

MSKKSIQKVYALTPMQEGMLYHAMLDPHSSSYFTQLELGIHGAFDLEIFEKSVNELIRSYD
ILRTVFVHQQLQKPRQVVLAERKTKVHYEDISHADENRQKEPHERYKQDVQRQGFNLAK
DILFKVAVFRLAADQLYLVWSNHHIMMDGWSMGVLMKSLFQNYEALRAGRTPANGQG
KPYSDYIKWLGKQDNEEAESYWSERLAGFEQPSVLPGRLPVKKDEYVNKEYSFTWDETL
VARIQQTANLHQVTGPNLFQAVWGIVLSKYNFTDDVIFGTVVSGRPSEINGIETMAGLFIN
TIPVRVKVERDAAFADIFTAVQQHAVEAERYDYVPLYEIQKRSALDGNLLNHLVAFENYP
LDQELENGSMEDRLGFSIKVESAFEQTSFDFNLIVYPGKTWTVKIKYNGAAFDSAFIERTA
EHLTRMMEAAVDQPAAFVREYGLVGDEEQRQIVEVFNSTKAELPEGMAVHQVFEEQAK
RTPASTAVVYEGTKLTYRELNAAANRLARKLVEHGLQKGETAAIMNDRSVETVVGMLA
VLKAGAAYVPLDPALPGDRLRFMAEDSSVRMVLIGNSYTGQAHQLQVPVLTLDIGFEESE
AADNLNLPSAPSDLAYIMYTSGSTGKPKGVMIEHKSILRLVKNAGYVPVTEEDRMAQTGA
VSFDAGTFEVFGALLNGAALYPVKKETLLDAKQFAAFLREQSITTMWLTSPLFNQLAAKD
AGMFGTLRHLIIGGDALVPHIVSKVKQASPSLSLWNGYGPTENTTFSTSFLIDREYGGSIPIG
KPIGNSTAYIMDEQQCLQPIGAPGELCVGGIGVARGYVNLPELTEKQFLEDPFRPGERIYRT
GDLARWLPDGNIEFLGRIDNQVKVRGFRIELGEIETKLNMAEHVTEAAVIIRKNKADENEI
CAYFTADREVAVSELRKTLSQSLPDYMVPAHLIQMDSLPLTPNGKINKKELPAPQSEAVQP
EYAAPKTESEKKLAEIWEGILGVKAGVTDNFFMIGGHSLKAMMMTAKIQEHFHKEVPIKV
LFEKPTIQELALYLEENESKEEQTFEPIRQASYQQHYPVSPAQRRMYILNQLGQANTSYNV
PAVLLLEGEVDKDRLENAIQQLINRHEILRTSFDMIDGEVVQTVHKNISFQLEAAKGREED
AEEIIKAFVQPFELNRAPLVRSKLVQLEEKRHLLLIDMHHIITDGSSTGILIGDLAKIYQGAD
LELPQIHYKDYAVWHKEQTNYQKDEEYWLDVFKGELPILDLPADFERPAERSFAGERVM
FGLDKQITAQIKSLMAETDTTMYMFLLAAFNVLLSKYASQDDIIVGSPTAGRTHPDLQGVP
GMFVNTVALRTAPAGDKTFAQFLEEVKTASLQAFEHQSYPLEELIEKLPLTRDTSRSPLFS
VMFNMQNMEIPSLRLGDLKISSYSMLHHVAKFDLSLEAVEREEDIGLSFDYATALFKDETI
RRWSRHFVNIIKAAAANPNVRLSDVDLLSSAETAALLEERHMTQITEATFAALFEKQAQQ
TPDHSAVKAGGNLLTYRELDEQANQLAHHLRAQGAGNEDIVATVMDRSAEVMVSILGV
MKAGAAFLPIDPDTPEERIRYSLEDSGAKFAVVNERNMTAIGQYEGIIVSLDDGKWRNESK
ERPSSISGSRNLAYVIYTSGTTGKPKGVQIEHRNLTNYVSWFSEEAGLTENDKTVLLSSYAF
DLGYTSMFPVLLGGGELHIVQKETYTAPDEIAHYIKEHGITYIKLTPSLFHTIVNTASFAKD
ANFESLRLIVLGGEKIIPTDVIAFRKMYGHTEFINHYGPTEATIGAIAGRVDLYEPDAFAKRP
TIGRPIANAGALVLNEALKLVPPGASGQLYITGQGLARGYLNRPQLTAERFVENPYSPGSL
MYKTGDVVRRLSDGTLAFIGRADDQVKIRGYRIEPKEIETVMLSLSGIQEAVVLAVSEGGL
QELCAYYTSDQDIEKAELRYQLSLTLPSHMIPAFFVQVDAIPLTANGKTDRNALPKPNAAQ
SGGKALAAPETALEESLCRIWQKTLGIEAIGIDDNFFDLGGHSLKGMMLIANIQAELEKSV
PLKALFEQPTVRQLAAYMEASAVSGGHQVLKPADKQDMYPLSSAQKRMYVLNQLDRQT
ISYNMPSVLLMEGELDISRLRDSLNQLVNRHESLRTSFMEANGEPVQRIIEKAEVDLHVFE
AKEDEADQKIKEFIRPFDLNDAPLIRAALLRIEAKKHLLLLDMHHIIADGVSRGIFVKELAL
LYKGEQLPEPTLHYKDFAVWQNEAEQKERMKEHEAYWMSVLSGELPELDLPLDYARPP
VQSFKGDTIRFRTGSETAKAVEKLLAETGTTLHMVLHAVFHVFLSKISGQRDIVIGSVTAG
RTNADVQDMPGMFVNTLALRMEAKEQQTFAELLELAKQTNLSALEHQEYPFEDLVNQL
DLPRDMSRNPLFNVMVTTENPDKEQLTLQNLSISPYEAHQGTSKFDLTLGGFTDENGIGLQ
LEYATDLFAKETAEKWSEYVLRLLKAVADNPNQPLSSLLLVTETEKQALLEAWKGKALP
VPTDKTVHQLFEETVQRHKDRPAVTYNGQSWTYGELNAKANRLARILMDCGISPDDRVG
VLTKPSLEMSAAVLGVLKAGAAFVPIDPDYPDQRIEYILQDSGAKLLLKQEGISVPDSYTG
DVILLDGSRTILSLPLDENDEGNPETAVTAENLAYMIYTSGTTGQPKGVMVEHHALVNLC
FWHHDAFSMTAEDRSAKYAGFGFDASIWEMFPTWTIGAELHVIDEAIRLDIVRLNDYFET
NGVTITFLPTQLAEQFMELENTSLRVLLTGGDKLKRAVKKPYTLVNNYGPTENTVVATSA
EIHPEEGSLSIGRAIANTRVYILGEGNQVQPEGVAGELCVAGRGLARGYLNREDETAKRFV
ADPFVPGERMYRTGDLVKWVNGGIEYIGRIDQQVKVRGYRIELSEIEVQLAQLSEVQDAA
VTAVKDKGGNTAIAAYVTPETADIEALKSTLKETLPDYMIPAFWVTLNELPVTANGKVDR
KALPEPDIEAGSGEYKAPTTDMEELLAGIWQDVLGMSEVGVTDNFFSLGGDSIKGIQMAS
RLNQHGWKLEMKDLFQHPTIEELTQYVERAEGKQADQGPVEGEVILTPIQRWFFEKNFTN
KHHWNQSVMLHAKKGFDPERVEKTLQALIEHHDALRMVYREENGDIVQVYKPIGESKVS
FEIVDLYGSDEEMLRSQIKLLANKLQSSLDLRNGPLLKAEQYRTEAGDHLLIAVHHLVVD
GVSWRILLEDFASGYMQAEKEESLVFPQKTNSFKDWAEELAAFSQSAHLLQQAEYWSQI
AAEQVSPLPKDCETEQRIVKDTSSVLCELTAEDTKHLLTDVHQPYGTEINDILLSALGLTM
KEWTKGAKIGINLEGHGREDIIPNVNISRTVGWFTAQYPVVLDISDADASAVIKTVKENLR
RIPDKGVGYGILRYFTETAETKGFTPEISFNYLGQFDSEVKTDFFEPSAFDMGRQVSGESEA
LYALSFSGMIRNGRFVLSCSYNEKEFERATVEEQMERFKENLLMLIRHCTEKEDKEFTPSD
FSAEDLEMDEMGDIFDMLEENLK
SrfAC

SEQ ID NO: 45

MSQFSKDQVQDMYYLSPMQEGMLFHAILNPGQSFYLEQITMKVKGSLNIKCCLEESMNVI
MDRYDVFRTVFIHEKVKRPVQVVLKKRQFHIEEIDLTHLTGSEQTAKINEYKEQDKIRGFD
LTRDIPMRAAIFKKAEESFEWWSYHHIILDGWCFGIVVQDLFKVYNALREQKPYSLPPV
KPYKDYIKWLEKQDKQASLRYWREYLEGFEGQTTFAEQRKKQKDGYEPKELLFSLSEAE
TKAFTELAKSQHTTLSTALQAVWSVLISRYQQSGDLAFGTVVSGRPAEIKGVEHMVGLFI
NVVPRRVKLSEGITFNGLLKRLQEQSLQSEPHQYVPLYDIQSQADQPKLIDHIIVFENYPLQ
DAKNEESSENGFDMVDVHVFEKSNYDLNLMASPGDEMLIKLAYNENVFDEAFILRLKSQ
LLTAIQQLIQNPDQPVSTINLVDDREREFLLTGLNPPAQAHETKPLTYWFKEAVNANPDAP
ALTYSGQTLSYRELDEEANRIARRLQKHGAGKGSVVALYTKRSLELVIGILGVLKAGAAY
LPVDPKLPEDRISYMLADSAAACLLTHQEMKEQAAELPYTGTTLFIDDQTRFEEQASDPAT
AIDPNDPAYIMYTSGTTGKPKGNITTHANIQGLVKHVDYMAFSDQDTFLSVSNYAFDAFT
FDFYASMLNAARLIIADEHTLLDTERLTDLILQENVNVMFATTALFNLLTDAGEDWMKGL
RCILFGGERASVPHVRKALRIMGPGKLINCYGPTEGTVFATAHVVHDLPDSISSLPIGKPISN
ASVYILNEQSQLQPFGAVGELCISGMGVSKGYVNRADLTKEKFIENPFKPGETLYRTGDLA
RWLPDGTIEYAGRIDDQVKIRGHRIELEEIEKQLQEYPGVKDAVVVADRHESGDASINAYL
VNRTQLSAEDVKAHLKKQLPAYMVPQTFTFLDELPLTTNGKVNKRLLPKPDQDQLAEEW
IGPRNEMEETIAQIWSEVLGRKQIGIHDDFFALGGHSLKAMTAASRIKKELGIDLPVKLLFE
APTIAGISAYLKNGGSDGLQDVTIMNQDQEQIIFAFPPVLGYGLMYQNLSSRLPSYKLCAF
DFIEEEDRLDRYADLIQKLQPEGPLTLFGYSAGCSLAFEAAKKLEEQGRIVQRIIMVDSYKK
QGVSDLDGRTVESDVEALMNVNRDNEALNSEAVKHGLKQKTHAFYSYYVNLISTGQVK
ADIDLLTSGADFDMPEWLASWEEATTGVYRVKRGFGTHAEMLQGETLDRNAEILLEFLN
TQTVTVS

SEQ ID NO: 46

gaattcgcatgcatcgacaaaaatgtcatgaaagaatcgttgtaagacgctcttcgcaagggtgtctttttttgcctttttttcggtttttgcgcggtaca
catagtcatgtaaagattgtaaattgcattcagcaataaaaaaagattgaacgcagcagtttggtttaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaagcagatgattctcaaaac
atggaacgccacaggaaaaacgtatccatatataacttttcatgagttgtttgagcagcaggcgaaaaagacgcctgatagagcggctgtcagct
atgaaggtcaaacattgacgtatcgggagcttgatgagaaaagcacacagctggccatttatttgcaggcgcatggagtgggtcctgaccgtctg
gcggggatctatgtggatcgatcgctggacatgctagtgggtttattagcgatcctcaaggctgggggagcgtatgtgccgctagacccgtccta
tccggctgaacgattagaatacatgcttgaggacagtgaagttttcattacactgacgacatcggaattagtaaatacgttgagttggaacggtgtc
acaacagcccttttagatcaagattgggatgaaattgctcaaacagcctctgatcgaaaagtgcttacacgcactgtcacgccagagaacttggc
atatgtcatttatacatccggcagcacaggaaagccaaaaggtgtcatgataccacataaagctttgacaaactttctcgtttcgatgggggaaaca
ccaggtcttacggcagaggataaaatgcttgctgtcacaacctactgttttgatattgcagctctggaattatttttgcctttaataaagggcgcacac
tgctatatttgtcaaacggagcatacaaaagacgttgaaaaactgaaacgggacatccgcgcgatcaaaccgacagtgatgcaggcaaccccc
gctacgtggaagatgctcttttattcaggctgggaaaatgaagagagcgtgaaaattttatgcggtggcgaagcattgcctgagacattaaaacga
tatttcttagatacgggcagcgaagcctggaatatgttcgggccaaccgaaacaactatctggtcagcggttcagcgcattaacgttgaatgctct
catgccacgataggaaggccaatcgccaatacacaaatctatattacggattctcagctcgcgccagtgccggcaggtgttccgggtgagctgt
gcattgcaggagacggtgtggcgaagggctactacaaaaaggaagaattaacggattcgagattcattgacaacccttttgagcctgggtctaa
gctttatagaacgggagacatggcccgttggcttacgggagggcgaattgaatatataggccgcatcgataatcaagtaaaaatccgcggattc
cgtattgaacttggtgatattgaaagcaggcttagtgagcatcccggcattctggaatgcgttgtggtcgcagatatggataacctagctgcctatta
tacagctaaacatgcaaatgcttctctcacagcgagagagctgcgtcattttgtgaaaaacgctttgcctgcctatatggtgccttcttattttattcag
cttgatcatatgccgttaactccgaacggaaagatagatagaaacagccttaagaatatcgatttatcaggggagcagctaaagcaaaggcaga
cctctcctaagaacattcaggatactgtttttaccatttggcaggaagtgctgaaaacgagtgacattgaatgggatgacttctttgcgctcggagg
gcattccttgaaggccatgaccgccgcgtcccgcatcaagaaagagctcgggattgatcttccagtgaagcttttgtttgaagcgccgacgatcg
ccggcatttcagcgtatttgaaaaacgggggctctgatggcttgcaggatgtaacgataatgaatcaggatcaggagcagatcattttcgcatttcc
gccggttctgggctatggccttatgtaccaaaatctgtccagccgcttgccgtcatacaagctatgcgcctttgattttattgaggaggaagaccgg
cttgaccgctatgcggatttgatccagaagctgcagccggaagggcctttaacattgtttggatattcagcgggatgcagcctggcgtttgaagct
gcgaaaaagcttgaggaacaaggccgtattgttcagcggatcatcatggtggattcctataaaaaacaaggtgtcagtgatctggacggacgca
cggttgaaagtgatgtcgaagcgttgatgaatgtcaatcgggacaatgaagcgctcaacagcgaagccgtcaaacacggcctcaagcaaaaa
acacatgccttttactcatactacgtcaacctgatcagcacaggccaggtgaaagcagatattgatctgttgacttccggcgctgattttgacatgcc
ggaatggcttgcatcatgggaagaagctacaacaggtgtttaccgtgtgaaaagaggcttcggaacacacgcagaaatgctgcagggcgaaa
cgctagataggaatgcggagattttgctcgaatttcttaatacacaaaccgtaacggtttcataaatgaagtgatgaaaggaggagacagccaatg
agccaactcttcaaatcatttgatgcgtcggaaaaaacacagctcatctgttttccgtttgccggcggctattcggcgtcgtttcgccctctccatgct
tttttgcagggggagtgcgagatgctcgctgccgagccgccgggacacggcacgaatcaaacgtcagccattgaggatctcgaagagctgac
ggatttgtacaagcaagaactgaaccttcgccctgatcggccgtttgtgctgttcggacacagtatgggcggaatgatcaccttcaggctggcgc
aaaagcttgagcgtgaaggcatctttccgcaggcggttatcatttctgcaatccagccgcctcatattcagcggaagaaagtgtcccacctgcctg
atgatcagtttctcgatcatattatccaattaggcggaatgcccgcagagcttgttgaaaataaggaggtcatgtcctttttcctgccttctttccgatc
agattaccgggctcttgaacaatttgagctttacgatctggcccagatccagtcgcctgttcatgtctttaacgggcttgatgataaaaaatgcatac
gagatgcggaagggtggaagaagtgggcaaaagacatcacattccatcaatttgacggcgggcacatgttcctgctgtcacaaacggaagaa
gtcgcagaacggatttttgcgatcttgaatcagcatccgatcattgsaaccgtgaagccgccccgcagggcgctccgcaggccgcttccggacca
ctccggaagcggccgtgcggtcggaggatcc

SEQ ID NO: 47

gaattcgcatgcatcgacaaaaatgtcatgaaagaatcgttgaagacgctcttcgcaagggtgtctttttttgcctttttttcggtttttgcgcggtaca
catagtcatgtaaagattgtaaattgcattcagcaataaaaaaagattgaacgcagcagtttggmaaaaatttttatttttctgtaaataatgtttagtg
gaaatgattgcggcatcccgcaaaaaatattgctgtaaataaactggaatctttcggcatcccgcatgaaacttttcacccatttttcggtgataaaa
acatttttttcatttaaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaagcagatgattctcaaaac
atggaacgccacaggaaaaacgtatccatatataacttttcatgagttgtttgagcagcaggcgaaaaagacgcctgatagagcggctgtcagct
atgaaggtcaaacattgacgtatcgggagcttgatgagaaaagcacacagctggccatttatttgcaggcgcatggagtgggtcctgaccgtctg
gcggggatctatgtggatcgatcgctggacatgctagtgggtttattagcgatcctcaaggctgggggagcgtatgtgccgctagacccgtccta
tccggctgaacgattagaatacatgcttgaggacagtgaagttttcattacactgacgacatcggaattagtaaatacgttgagttggaacggtgtc
acaacagcccttttagatcaagattgggatgaaattgctcaaacagcctctgatcgaaaagtgcttacacgcactgtcacgccagagaacttggc
atatgtcatttatacatccggcagcacaggaaagccaaaaggtgtcatgataccacataaagctttgacaaactttctcgtttcgatgggggaaaca
ccaggtcttacggcagaggataaaatgcttgctgtcacaacctactgttttgatattgcagctctggaattatttttgcctttaataaagggcgcacac
tgctatatttgtcaaacggagcatacaaaagacgttgaaaaactgaaacgggacatccgcgcgatcaaaccgacagtgatgcaggcaaccccc
gctacgtggaagatgctcttttattcaggctgggaaaatgaagagagcgtgaaaattttatgcggtggcgaagcattgcctgagacattaaaacga
tatttcttagatacgggcagcgaagcctggaatatgttcgggccaaccgaaacaactatctggtcagcggttcagcgcattaacgttgaatgctct
catgccacgataggaaggccaatcgccaatacacaaatctatattacggattctcagctcgcgccagtgccggcaggtgttccgggtgagctgt
gcattgcaggagacggtgtggcgaagggctactacaaaaaggaagaattaacggattcgagattcattgacaacccttttgagcctgggtctaa
gctttatagaacgggagacatggcccgttggcttacgggagggcgaattgaatatataggccgcatcgataatcaagtaaaaatccgcggattc
cgtattgaacttggtgatattgaaagcaggcttagtgagcatcccggcattctggaatgcgttgtggtcgcagatatggataacctagctgcctatta
tacagctaaacatgcaaatgcttctctcacagcgagagagctgcgtcattttgtgaaaaacgctttgcctgcctatatggtgccttcttattttattcag
cttgatcatatgccgttaactccgaacggaaagatagatagaaacagccttaagaatatcgatttatcaggggagcagctaaagcaaaggcaga
cctctcctaagaacattcaggatactgtttttaccatttggcaggaagtgctgaacgttgagaaggcggggatctttgacttctttgcgctcggagg
gcattccttgaaggccatgaccgccgcgtcccgcatcaagaaagagctcgggattgatcttccagtgaagcttttgtttgaagcgccgacgatcg
ccggcatttcagcgtatttgaaaaacgggggctctgatggcttgcaggatgtaacgataatgaatcaggatcaggagcagatcattttcgcatttcc
gccggttctgggctatggccttatgtaccaaaatctgtccagccgcttgccgtcatacaagctatgcgcctttgattttattgaggaggaagaccgg
cttgaccgctatgcggatttgatccagaagctgcagccggaagggcctttaacattgtttggatattcagcgggatgcagcctggcgtttgaagct
gcgaaaaagcttgaggaacaaggccgtattgttcagcggatcatcatggtggattcctataaaaaacaaggtgtcagtgatctggacggacgca
cggttgaaagtgatgtcgaagcgttgatgaatgtcaatcgggacaatgaagcgctcaacagcgaagccgtcaaacacggcctcaagcaaaaa
acacatgccttttactcatactacgtcaacctgatcagcacaggccaggtgaaagcagatattgatctgttgacttccggcgctgattttgacatgcc
ggaatggcttgcatcatgggaagaagctacaacaggtgtttaccgtgtgaaaagaggcttcggaacacacgcagaaatgctgcagggcgaaa
cgctagataggaatgcggagattttgctcgaatttcttaatacacaaaccgtaacggtttcataaatgaagtgatgaaaggaggagacagccaatg
agccaactcttcaaatcatttgatgcgtcggaaaaaacacagctcatctgttttccgtttgccggcggctattcggcgtcgtttcgccctctccatgct
tttttgcagggggagtgcgagatgctcgctgccgagccgccgggacacggcacgaatcaaacgtcagccattgaggatctcgaagagctgac
ggatttgtacaagcaagaactgaaccttcgccctgatcggccgtttgtgctgttcggacacagtatgggcggaatgatcaccttcaggctggcgc
aaaagcttgagcgtgaaggcatctttccgcaggcggttatcatttctgcaatccagccgcctcatattcagcggaagaaagtgtcccacctgcctg
atgatcagtttctcgatcatattatccaattaggcggaatgcccgcagagcttgttgaaaataaggaggtcatgtcctttttcctgccttctttccgatc
agattaccgggctcttgaacaatttgagctttacgatctggcccagatccagtcgcctgttcatgtctttaacgggcttgatgataaaaaatgcatac
gagatgcggaagggtggaagaagtgggcaaaagacatcacattccatcaatttgacggcgggcacatgttcctgctgtcacaaacggaagaa
gtcgcagaacggatttttgcgatcttgaatcagcatccgatcattcaaccgtgaagccgccccgcagggcgctccgcaggccgcttccggacca
ctccggaagcggccgtgcggtcggaggatcc

SEQ ID NO: 48

gaattcgcatgcatcgacaaaaatgtcatgaaagaatcgttgtaagacgctcttcgcaagggtgtcttttmgcctttttttcggtttttgcgcggtaca
catagtcatgtaaagattgtaaattgcattcagcaataaaaaaagattgaacgcagcagtttggtttaaaaatttttaltmctgtaaataatgtttagtg
gaaatgattgcggcatcccgcaaaaaatattgctgtaaataaactggaatctttcggcatcccgcatgaaacttttcacccatttttcggtgataaaa
acatttttttcatltcaactgaacggtagaaagataaaaaatattgaaaacaatgaataaatagccaaaattggtttcttattagggtggggtcttgcgg
tctttatccgcttatgttaaacgccgcaatgctgactgacggcagcctgctttaatagcggccatctgttttttgattggaagcactgctttttaagtgta
gtactttgggctatttcggctgttagttcataagaattaaaagctgatatggataagaaagagaaaatgcgttgcacatgttcactgcttataaagatt
aggggaggtatgacaatatggaaataactttttaccctttaacggatgcacaaaaacgaatttggtacacagaaaaattttatcctcacacgagcatt
tcaaatcttgcggggattggtaagctggtttcagctgatgcgattgattatgtgcttgttgagcaggcgattcaagagtttattcgcagaaatgacgc
catgcgccttcggttgcggctagatgaaaacggggagcctgttcaatatattagcgagtatcggcctgttgatataaaacatactgacactactga
agatccgaatgcgatagagtttatttcacaatggagccgggaggaaacgaagaaacctttgccgctatacgattgtgatttgttccgtttttccttgtt
caccataaaggaaaatgaagtgtggttttacgcaaatgttcatcacgtgatttctgatggtatctccatgaatattctcgggaatgcgatcatgcacat
ttatttagaattagccagcggctcagagacaaaagaaggaatctcgcattcatttatcgatcatgttttatctgaacaggaatatgctcaatcgaagc
ggtttgaaaaggacaaggcgttttggaacaaacaatttgaatcggtgcctgaacttgtttccttgaaacggaatgcatccgcagggggaagtttag
atgctgagaggttctctaaagatgtgcctgaagcgcttcatcagcagattctgtcgttttgtgaggcgaataaagtcagtgttctttcggtatttcaatc
gctgctcgccgcctatttgtacagggtcagcggccagaatgatgttgtgacgggaacatttatgggcaaccggacaaatgcgaaagagaagca
gatgcttggcatgtttgtttctacggttccgcttcggacaaacattgacggcgggcaggcgttttcagaatttgtcaaagaccggatgaaggatctg
atgaagacacttcgccaccaaaagtatccgtataatctcctaatcaacgatttgcgtgaaacaaagagctctctgaccaagctgttcacggtttctct
tgaatatcaagtgatgcagtggcagaaagaagaggatcttgcctttttgactgagccgattttcagcggcagcggattaaatgatgtctcaattcat
gtaaaggatcgatgggatactgggaaactcaccatagattttgattaccgcactgatttattttcacgtgaagaaatcaacatgatttgtgagcgcat
gattaccatgctggagaacgcgttaacgcatccagaacatacaattgatgaattaacactgatttctgatgcggagaaagaaaaagtcatttctaaa
tggaatgagacggcaaaatccgagaagctggtcagccttcaggacatgttcgaaaagcaggcagttcttacgccagaacgtatcgctctcatgt
gtgatgacattcaagtcaactatcgaaagctcaatgaagaggcaaaccgcctcgcgcgtctgttgatcgaaaaagggattggtccggagcaattt
gtcgctttggcgctgccgcgttcccctgagatggtggcttcaatgcttggtgtgctcaagactggtgcggcgtatctcccccttgatccggagtttc
cagccgaccgcatttcttacatgctggaggatgcgaaaccttcatgcatcataacgactgaggaaatagcagccagtctgcctgatgacctggct
gtacctgagcttgtgcttgatcaggctgttacacaggagattataaaacgctattcgccggaaaatcaggatgtatcggtttcgcttgaccatcctgc
gtatatcatctatacctcaggatcaacaggaagaccgaagggagtcgttgtgacacagaaaagcttaagcaattttctgctgtctatgcaggaggc
attttctctaggagaagaagacaggctgttggctgtgacgactgtcgcttttgatatttcagcattggagttatatcttccgctgattagcggagcgca
aatcgtgatcgcaaagaaagaaacgatccgtgagccgcaggcattagctcaaatgattgaaaatttcgatattaacattatgcaggcgacaccga
cactgtggcacgctttggtaacgagtgaacctgagaaacttcgggggcttagagtgcttgtaggaggcgaggcgctgccgagcggtcttttgca
ggaacttcaagaccttcattgttcagtcacgaacttatacggtcccactgaaacaacgatttggtctgccgcagcttttcttgaagaagggctgaag
ggcgttcctccgattgggaaaccgatttggaacacgcaggtgtatgtgcttgataatggattgcagccggtgccgccgggcgttgtcggagagc
tttatattgcaggaaccggcttggccagaggttatttccatcgtcctgatttaacggcggagcgcttcgttgcagatccatacggaccgccgggaa
ctcggatgtatcggaccggagaccaggcccgctggcgcgccgatgggtctttggattatatcgggcgggcggatcatcaaatcaaaattcgcg
gattccgaattgaacttggagaaattgatgccgtgcttgccaatcacccgcacattgaacaggccgcggttgtcgtacgggaagatcagccggg
agacaaacgattggcggcttatgtagtcgctgatgctgctattgatactgcagagcttcgtcgttatatgggggctagtcttcctgattatatggtacc
gtcggcgtttgtggagatggacgagctgccgttaacacctaatggcaagcttgaccggaaagcactgccggcaccagacttcagcacatctgtc
agtgatcgggccccgcggactcctcaggaagagatattgtgtgacttgtttgcagaggttctcggtttggcacgcgtcggtatcgatgacttctttg
cgctcggagggcattccttgaaggccatgaccgccgcgtcccgcatcaagaaagagctcgggattgatcttccagtgaagcttttgtttgaagcg
ccgacgatcgccggcatttcagcgtatttgaaaaacgggggctctgatggcttgcaggatgtaacgataatgaatcaggatcaggagcagatca
ttttcgcatttccgccggttctgggctatggccttatgtaccaaaatctgtccagccgcttgccgtcatacaagctatgcgcctttgattttattgagga
ggaagaccggcttgaccgctatgcggatttgatccagaagctgcagccggaagggcctttaacattgtttggatattcagcgggatgcagcctg
gcgtttgaagctgcgaaaaagcttgaggaacaaggccgtattgttcagcggatcatcatggtggattcctataaaaaacaaggtgtcagtgatctg
gacggacgcacggttgaaagtgatgtcgaagcgttgatgaatgtcaatcgggacaatgaagcgctcaacagcgaagccgtcaaacacggcct
caagcaaaaaacacatgccttttactcatactacgtcaacctgatcagcacaggccaggtgaaagcagatattgatctgttgacttccggcgctga
ttttgacatgccggaatggcttgcatcatgggaagaagctacaacaggtgtttaccgtgtgaaaagaggcttcggaacacacgcagaaatgctg
cagggcgaaacgctagataggaatgcggagattttgctcgaatttcttaatacacaaaccgtaacggtttcataaatgaagtgatgaaaggagga
gacagccaatgagccaactcttcaaatcatttgatgcgtcggaaaaaacacagctcatctgttttccgtttgccggcggctattcggcgtcgtttcg
ccctctccatgcttttttgcagggggagtgcgagatgctcgctgccgagccgccgggacacggcacgaatcaaacgtcagccattgaggatctc
gaagagctgacggatttgtacaagcaagaactgaaccttcgccctgatcggccgtttgtgctgttcggacacagtatgggcggaatgatcacctt
caggctggcgcaaaagcttgagcgtgaaggcatctttccgcaggcggttatcatttctgcaatccagccgcctcatattcagcggaagaaagtgt
cccacctgcctgatgatcagtttctcgatcatattatccaattaggcggaatgcccgcagagcttgttgaaaataaggaggtcatgtcctttttcctgc
cttctttccgatcagattaccgggctcttgaacaatttgagctttacgatctggcccagatccagtcgcctgttcatgtctttaacgggcttgatgataa
aaaatgcatacgagatgcggaagggtggaagaagtgggcaaaagacatcacattccatcaatttgacggcgggcacatgttcctgctgtcaca
aacggaagaagtcgcagaacggatttttgcgatcttgaatcagcatccgatcattcaaccgtgaagccgccccgcagggcgctccgcaggccg
cttccggaccactccggaagcggccgtgcggtcggaggatcc
(A-domain BSU)

SEQ ID NO: 49

gagaagcagatgattctcaaaacatggaacgccacaggaaaaacgtatccatatataacttttcatgagttgtttgagcagcaggcgaaaaagac
gcctgatagagcggctgtcagctatgaaggtcaaacattgacgtatcgggagcttgatgagaaaagcacacagctggccatttatttgcaggcg
catggagtgggtcctgaccgtctggcggggatctatgtggatcgatcgctggacatgctagtgggtttattagcgatcctcaaggctgggggag
cgtatgtgccgctagacccgtcctatccggctgaacgattagaatacatgcttgaggacagtgaagttttcattacactgacgacatcggaattagt
aaatacgttgagttggaacggtgtcacaacagcccttttagatcaagattgggatgaaattgctcaaacagcctctgatcgaaaagtgcttacacg
cactgtcacgccagagaacttggcatatgtcatttatacatccggcagcacaggaaagccaaaaggtgtcatgataccacataaagctttgacaa
actttctcgtttcgatgggggaaacaccaggtcttacggcagaggataaaatgcttgctgtcacaacctactgttttgatattgcagctctggaattat
ttttgcctttaataaagggcgcacactgctatatttgtcaaacggagcatacaaaagacgttgaaaaactgaaacgggacatccgcgcgatcaaa
ccgacagtgatgcaggcaacccccgctacgtggaagatgctcttttattcaggctgggaaaatgaagagagcgtgaaaattttatgcggtggcg
aagcattgcctgagacattaaaacgatatttcttagatacgggcagcgaagcctggaatatgttcgggccaaccgaaacaactatctggtcagcg
gttcagcgcattaacgttgaatgctctcatgccacgataggaaggccaatcgccaatacacaaatctatattacggattctcagctcgcgccagtg
ccggcaggtgttccgggtgagctgtgcattgcaggagacggtgtggcgaagggctactacaaaaaggaagaattaacggattcgagattcatt
gacaacccttttgagcctgggtctaagctttatagaacgggagacatggcccgttggcttacgggagggcgaattgaatatataggccgcatcga
taatcaagtaaaaatccgcggattccgtattgaacttggtgatattgaaagcaggcttagtgagcatcccggcattctggaatgcgttgtggtcgca
gatatggataacctagctgcctattatacagctaaacatgcaaatgcttctctcacagcgagagagctgcgtcattttgtgaaaaacgctttgcctgc
ctatatggtgccttcttattttattcagcttgatcatatgccgttaactccgaacggaaagatagatagaaacagccttaagaatatcgatttatcaggg
gagcagctaaagcaaaggcagacctct
(C-domain)

SEQ ID NO: 50

Cctttaacggatgcacaaaaacgaatttggtacacagaaaaattttatcctcacacgagcatttcaaatcttgcggggattggtaagctggtttcag
ctgatgcgattgattatgtgcttgttgagcaggcgattcaagagtttattcgcagaaatgacgccatgcgccttcggttgcggctagatgaaaacgg
ggagcctgttcaatatattagcgagtatcggcctgttgatataaaacatactgacactactgaagatccgaatgcgatagagtttatttcacaatgga
gccgggaggaaacgaagaaacctttgccgctatacgattgtgatttgttccgtttttccttgttcaccataaaggaaaatgaagtgtggttttacgca
aatgttcatcacgtgatttctgatggtatctccatgaatattctcgggaatgcgatcatgcacatttatttagaattagccagcggctcagagacaaaa
gaaggaatctcgcattcatttatcgatcatgttttatctgaacaggaatatgctcaatcgaagcggtttgaaaaggacaaggcgttttggaacaaac
aatttgaatcggtgcctgaacttgtttccttgaaacggaatgcatccgcagggggaagtttagatgctgagaggttctctaaagatgtgcctgaagc
gcttcatcagcagattctgtcgttttgtgaggcgaataaagtcagtgttctttcggtatttcaatcgctgctcgccgcctatttgtacagggtcagcgg
ccagaatgatgttgtgacgggaacatttatgggcaaccggacaaatgcgaaagagaagcagatgcttggcatgtttgtttctacggttccgcttcg
gacaaacattgacggcgggcaggcgttttcagaatttgtcaaagaccggatgaaggatctgatgaagacacttcgccaccaaaagtatccgtat
aatctcctaatcaacgatttgcgtgaaacaaagagctctctgaccaagctgttcacggtttctcttgaatatcaagtgatgcagtggcagaaagaag
aggatcttgcctttttgactgagccgattttcagcggcagcggattaaatgatgtctcaattcatgtaaaggatcgatgggatactgggaaactcac
catagattttgattaccgcactgatttattttcacgtgaagaaatcaacatgatttgtgagcgcatgattaccatgctggagaacgcgttaacgcatcc
agaacatacaattgatgaatta
(PCP TE-domain)

SEQ ID NO: 51

Ttctttgcgctcggagggcattccttgaaggccatgaccgccgcgtcccgcatcaagaaagagctcgggattgatcttccagtgaagcttttgttt
gaagcgccgacgatcgccggcatttcagcgtatttgaaaaacgggggctctgatggcttgcaggatgtaacgataatgaatcaggatcaggag
cagatcattttcgcatttccgccggttctgggctatggccttatgtaccaaaatctgtccagccgcttgccgtcatacaagctatgcgcctttgatttta
ttgaggaggaagaccggcttgaccgctatgcggatttgatccagaagctgcagccggaagggcctttaacattgtttggatattcagcgggatgc
agcctggcgtttgaagctgcgaaaaagcttgaggaacaaggccgtattgttcagcggatcatcatggtggattcctataaaaaacaaggtgtcag
tgatctggacggacgcacggttgaaagtgatgtcgaagcgttgatgaatgtcaatcgggacaatgaagcgctcaacagcgaagccgtcaaaca
cggcctcaagcaaaaaacacatgccttttactcatactacgtcaacctgatcagcacaggccaggtgaaagcagatattgatctgttgacttccgg
cgctgattttgacatgccggaatggcttgcatcatgggaagaagctacaacaggtgtttaccgtgtgaaaagaggcttcggaacacacgcagaa
atgctgcagggcgaaacgctagataggaatgcggagattttgctcgaatttcttaatacacaaaccgtaacggtttcataa
(A-domain DHB)

SEQ ID NO: 52

Gagaaagaaaaagtcatttctaaatggaatgagacggcaaaatccgagaagctggtcagccttcaggacatgttcgaaaagcaggcagttctta
cgccagaacgtatcgctctcatgtgtgatgacattcaagtcaactatcgaaagctcaatgaagaggcaaaccgcctcgcgcgtctgttgatcgaa
aaagggattggtccggagcaatttgtcgctttggcgctgccgcgttcccctgagatggtggcttcaatgcttggtgtgctcaagactggtgcggc
gtatctcccccttgatccggagtttccagccgaccgcatttcttacatgctggaggatgcgaaaccttcatgcatcataacgactgaggaaatagc
agccagtctgcctgatgacctggctgtacctgagcttgtgcttgatcaggctgttacacaggagattataaaacgctattcgccggaaaatcagga
tgtatcggtttcgcttgaccatcctgcgtatatcatctatacctcaggatcaacaggaagaccgaagggagtcgttgtgacacagaaaagcttaag
caattttctgctgtctatgcaggaggcattttctctaggagaagaagacaggctgttggctgtgacgactgtcgcttttgatatttcagcattggagtt
atatcttccgctgattagcggagcgcaaatcgtgatcgcaaagaaagaaacgatccgtgagccgcaggcattagctcaaatgattgaaaatttcg
atattaacattatgcaggcgacaccgacactgtggcacgctttggtaacgagtgaacctgagaaacttcgggggcttagagtgcttgtaggaggc
gaggcgctgccgagcggtcttttgcaggaacttcaagaccttcattgttcagtcacgaacttatacggtcccactgaaacaacgatttggtctgcc
gcagcttttcttgaagaagggctgaagggcgttcctccgattgggaaaccgatttggaacacgcaggtgtatgtgcttgataatggattgcagcc
ggtgccgccgggcgttgtcggagagctttatattgcaggaaccggcttggccagaggttatttccatcgtcctgatttaacggcggagcgcttcgt
tgcagatccatacggaccgccgggaactcggatgtatcggaccggagaccaggcccgctggcgcgccgatgggtctttggattatatcgggc
gggcggatcatcaaatcaaaattcgcggattccgaattgaacttggagaaattgatgccgtgcttgccaatcacccgcacattgaacaggccgc
ggttgtcgtacgggaagatcagccgggagacaaacgattggcggcttatgtagtcgctgatgctgctattgatactgcagagcttcgtcgttatat
gggggctagtcttcctgattatatggtaccgtcggcgtttgtggagatggacgagctgccgttaacacctaatggcaagcttgaccggaaagcac
tgccggcaccagacttcagcacatctgtcagtgatcgggcc

SEQ ID NO: 53

SDAEKQM

SEQ ID NO: 54

TLISDAEK

SEQ ID NO: 55

IEWDDDFFAL

SEQ ID NO: 56

RVGIDDDFFALG

SEQ ID NO: 57

WQEVLNVEKAGIF

SEQ ID NO: 58

KLLARAGGKSVSYRKDMTIPELFQEKAELLSDHPAVVFEDRTLSYRTLHEQSARIANVLK
QKGVGPDSPVAVLIERSERMITAIMGILKAGGAYVPIDPGFPAERIQYILEDCGADFILTESK
VAAPEADAELIDLDQAIEEGAEESLNADVNARNLAYIIYTSGTTGRPKGVMIEHRQVHHL
VESLQQTIYQSGSQTLRMALLAPFHFDASVKQIFASLLLGQTLYIVPKKTVTNGAALTAYY
RKNSIEATDGTPAHLQMLAAAGDFEGLKLKHMLIGGEGLSSVVADKLLKLFKEAGTAPRL
TNVYGPTETCVDASVHPVIPENAVQSAYVPIGKALGNNRLYILDQKGRLQPEGVAGELYI
AGDGVGRGYLHLPELTEEKFLQDPFVPGDRMYRTGDVVRWLPDGTIEYLGREDDQVKVR
GYRIELGEIEAVIQQAPDVAKAVVLARPDEQGNLEVCAYVVQKPGSEFAPAGLREHAARQ
LPDYMVPAYFTEVTEIPLTPSGKVDRRKLFALEVKA

SEQ ID NO: 59

SQIQQFNPGRTPYPKDKTIVQLFEEQAANTPDHTALQYEGESLTYRELNERANRLARGILS
LGAGEGRTAAVLCERSMDMIVSILAVLKSGSAYVPIDPEHPIQRMQHFFRDSGAKVLLTQ
RKLKALAEEAEFKGVIVLADEEESYHADARNLALPLDSAAMANLTYTSGTTGTPKGNIVT
HANILRTVKETNYLSITEQDTILGLSNYVFDAFMFDMFGSLLNGAKLVLIPKETVLDMARL
SRVIERENISILMITTALFHLLVDLNPACLSTLRKIMFGGERASVEHVRKALQTVGKGKLLH
MYGPSESTVFATYHPVDELEEHTLSVPIGKPVSNTEVYILDRTGHVQPAGIAGELCVSGEG
LVKGYYNRPELTEEKFVPHPFTSGERMYKTGDLARWLPNGDIEFIGRIDHQVKIRGQRIEL
GEIEHQLQTHDRVQESVVLAVDQGAGDKLLCAYYVGEGDISSQEMREHAAKDLPAYMV
PAVFIQMDELPLTGNGKIDRRALPIPDANV

SEQ ID NO: 60

ALLEAWKGKALPVPTDKTVHQLFEETAQRHKDRPAVTYNGQSWTYGELNAKANRLARI
LMDCGISPDDRVGVLTKPSLEMSAAVLGVLKAGAAFVPIDPDYPDQRIEYILQDSGAKLLL
KQEGISVPDSYTGDVILLDGSRTILSLPLDENDEENPETAVTAENLAYMIYTSGTTGQPKGV
MVEHHALVNLCFWHHDAFSMTAEDRSAKYAGFGFDASIWEMFPTWTIGAELHVIEEAIR
LDIVRLNDYFETNGVTITFLPTQLAEQFMELENTSLRVLLTGGDKLKRAVKKPYTLVNNY
GPTENTVVATSAEIHPEEGSLSIGRAIANTRVYILGEGNQVQPEGVAGELCVAGRGLARGY
LNREDETAKRFVADPFVPGERMYRTGDLVKWTGGGIEYIGRIDQQVKVRGYRIELSEIEV
QLAQLSEVQDAAVTAVKDKGGNTAIAAYVTPESADIEALKSALKETLPDYMIPAFWVTLN
ELPVTANGKVDRKALPEPDIEA

SEQ ID NO: 61

QIVEVFNSTKAELPEGMAVHQVFEEQAKRTPASTAVVYEGTKLTYRELNAAANRLARKL
VEHGLQKGETAAIMNDRSVETVVGMLAVLKAGAAYVPLDPALPGDRLRFMAEDSSVRM
VLIGNSYTGQAHQLQVPVLTLDIGFEESEAADNLNLPSAPSDLAYIMYTSGSTGKPKGVMI
EHKSILRLVKNAGYVPVTEEDRMAQTGAVSFDAGTFEVFGALLNGAALYPVKKETLLDA
KQFAAFLREQSITTMWLTSPLFNQLAAKDAGMFGTLRHLIIGGDALVPHIVSKVKQASPSL
SLWNGYGPTENTTFSTSFLIDREYGGSIPIGKPIGNSTAYIMDEQQCLQPIGAPGELCVGGIG
VARGYVNLPELTEKQFLEDPFRPGERIYRTGDLARWLPDGNIEFLGRIDNQVKVRGFRIEL
GEIETKLNMAEHVTEAAVIIRKNKADENEICAYFTADREVAVSELRKTLSQSLPDYMVPA
HLIQMDSLPLTPNGKINKKELPAPQSEA

SEQ ID NO: 62

ALLEERHMTQITEATFAALFEKQAQQTPDHSAVKAGGNLLTYRELDEQANQLAHHLRAQ
GAGNEDIVAIVMDRSAEVMVSILGVMKAGAAFLPIDPDTPEERIRYSLEDSGAKFAVVNE
RNMTAIGQYEGIIVSLDDGKWRNESKERPSSISGSRNLAYVIYTSGTTGKPKGVQIEHRNLT
NYVSWFSEEAGLTENDKTVLLSSYAFDLGYTSMFPVLLGGGELHIVQKETYTAPDEIAHYI
KEHGITYIKLTPSLFHTIVNTASFAKDANFESLRLIVLGGEKIIPTDVIAFRKMYGHTEFINH
YGPTEATIGAIAGRVDLYEPDAFAKRPTIGRPIANAGALVLNEALKLVPPGASGQLYITGQ
GLARGYLNRPQLTAERFVENPYSPGSLMYKTGDVVRRLSDGTLAFIGRADDQVKIRGYRI
EPKEIETVMLSLSGIQEAVVLAVSEGGLQELCAYYTSDQDIEKAELRYQLSLTLPSHMIPAF
FVQVDAIPLTANGKTDRNALPKPNAAQ

SEQ ID NO: 63

ALLEAWKGKALPVPTDKTVHQLFEETVQRHKDRPAVTYNGQSWTYGELNAKANRLARI
LMDCGISPDDRVGVLTKPSLEMSAAVLGVLKAGAAFVPIDPDYPDQRIEYILQDSGAKLLL
KQEGISVPDSYTGDVILLDGSRTILSLPLDENDEGNPETAVTAENLAYMIYTSGTTGQPKG
VMVEHHALVNLCFWHHDAFSMTAEDRSAKYAGFGFDASIWEMFPTWTIGAELHVIDEAI
RLDIVRLNDYFETNGVTITFLPTQLAEQFMELENTSLRVLLTGGDKLKRAVKKPYTLVNN
YGPTENTVVATSAEIHPEEGSLSIGRAIANTRVYILGEGNQVQPEGVAGELCVAGRGLARG
YLNREDETAKRFVADPFVPGERMYRTGDLVKWVNGGIEYIGRIDQQVKVRGYRIELSEIE
VQLAQLSEVQDAAVTAVKDKGGNTAIAAYVTPETADIEALKSTLKETLPDYMIPAFWVTL
NELPVTANGKVDRKALPEPDIEA

SEQ ID NO: 64

FLLTGLNPPAQAHETKPLTYWFKEAVNANPDAPALTYSGQTLSYRELDEEANRIARRLQK
HGAGKGSVVALYTKRSLELVIGILGVLKAGAAYLPVDPKLPEDRISYMLADSAAACLLTH
QEMKEQAAELPYTGTTLFIDDQTRFEEQASDPATAIDPNDPAYIMYTSGTTGKPKGNITTH
ANIQGLVKHVDYMAFSDQDTFLSVSNYAFDAFTFDFYASMLNAARLIIADEHTLLDTERL
TDLILQENVNVMFATTALFNLLTDAGEDWMKGLRCILFGGERASVPHVRKALRIMGPGK
LINCYGPTEGTVFATAHVVHDLPDSISSLPIGKPISNASVYILNEQSQLQPFGAVGELCISGM
GVSKGYVNRADLTKEKFIENPFKPGETLYRTGDLARWLPDGTIEYAGRIDDQVKIRGHRIE
LEEIEKQLQEYPGVKDAVVVADRHESGDASINAYLVNRTQLSAEDVKAHLKKQLPAYMV
PQTFTFLDELPLTTNGKVNKRLLPKPDQDQ

SEQ ID NO: 65

KVISKWNETAKSEKLVSLQDMFEKQAVLTPERIALMCDDIQVNYRKLNEEANRLARLLIE
KGIGPEQFVALALPRSPEMVASMLGVLKTGAAYLPLDPEFPADRISYMLEDAKPSCIITTEE
IAASLPDDLAVPELVLDQAVTQEIIKRYSPENQDVSVSLDHPAYIIYTSGSTGRPKGVVVTQ
KSLSNFLLSMQEAFSLGEEDRLLAVTTVAFDISALELYLPLISGAQIVIAKKETIREPQALAQ
MIENFDINIMQATPTLWHALVTSEPEKLRGLRVLVGGEALPSGLLQELQDLHCSVTNLYGP
TETTIWSAAAFLEEGLKGVPPIGKPIWNTQVYVLDNGLQPVPPGVVGELYIAGTGLARGYF
HRPDLTAERFVADPYGPPGTRMYRTGDQARWRADGSLDYIGRADHQIKIRGFRIELGEID
AVLANHPHIEQAAVVVREDQPGDKRLAAYVVADAAIDTAELRRYMGASLPDYMVPSAF
VEMDELPLTPNGKLDRKALPAPDFST

SEQ ID NO: 66

ACLPEQFEKQAALRPDAIAVVYENQELSYAELNERANRLARMMISEGVGPEQFVALALPR
SLEMAVGLLAVLKAGAAYLPLDPDYPADRIAFMLKDAQPAFIMTNTKAANHIPPVENVPK
IVLDDPELAEKLNTYPAGNPKNKDRTQPLSPLNTAYVIYTSGSTGVPKGVMIPHQNVTRLF
AATEHWFRFSSGDIWTMFHSYAFDFSVWEIWGPLLHGGRLVIVPHHVSRSPEAFLRLLVK
EGVTVLNQTPSAFYQFMQAEREQPDLGQALSLRYVIFGGEALELSRLEDWYNRHPENRPQ
LINMYGITETTVHVSYIELDRSMAALRANSLIGCGIPDLGVYVLDERLQPVPPGVAGELYV
SGAGLARGYLGRPGLTSERFIADPFGPPGTRMYRTGDVARLRADGSLDYVGRADHQVKIR
GFRIELGEIEAALVQHPQLEDAAVIVREDQPGDKRLAAYVIPSEETFDTAELRRYAAERLP
DYMVPAAFVTMKELPLTPNGKLDRKALPAPDFA

SEQ ID NO: 67

LTDAQKRIWYTEKFYPHTSISNLAGIGKLVSADAIDYVLVEQAIQEFIRRNDAMRLRLRLD
ENGEPVQYISEYRPVDIKHTDTTEDPNAIEFISQWSREETKKPLPLYDCDLFRFSLFTIKENE
VWFYANVHHVISDGISMNILGNAIMHIYLELASGSETKEGISHSFIDHVLSEQEYAQSKRFE
KDKAFWNKQFESVPELVSLKRNASAGGSLDAERFSKDVPEALHQQILSFCEANKVSVLSV
FQSLLAAYLYRVSGQNDVVTGTFMGNRTNAKEKQMLGMFVSTVPLRTNIDGGQAFSEFV
KDRMKDLMKTLRHQKYPYNLLINDLRETKSSLTKLFTVSLEYQVMQWQKEEDLAFLTEPI
FSGSGLNDVSIHVKDRWDTGKLTIDFDYRTDLFSREEINMICERMITMLENALTHPEHTIDE
LTLI

SEQ ID NO: 68

FVLENSTPRQKIHDSVDFNIEMIERGGRSDEAIMASFVRTFDLAKAPLFRIGLLGLEENRHM
LLFDMHHLISDGVSIGIMLEELARIYKGEQLPDLRLQYKDYAVWQSRQAAEGYKKDQAY
WKEVFAGELPVLQLLSDYPRPPVQSFEGDRVSIKLDAGVKDRLNRLAEQNGATLYMVML
SAYYTLLSKYTGQDDIIVGTPSAGRNHSDTEGIIGMFVNTLAIRSEVKQNETFTQLISRVRK
RVLDAFSHQDYPFEWLVEDLNIPRDVSRHPLFDTMFSLQNATEGIPAVGDLSLSVQETNFK
IAKFDLTVQARETDEGIEIDVDYSTKLFKQSTADRLLTHFARLLEDAAADPEKPISEYKLL

SEQ ID NO: 69

VSSPQKRMYVLQQLEDAQTSYNMPAVLRLTGELDVERLNSVMQQLMQRHEALRTTFEIK
DGETVQRIWEEAECEIAYFEAPEEETERIVSEFIKPFKIDQLPLFRIGLIKHSDTEHVLLFDMH
HIISDGASVGVLIEELSKLYDGETLEPLRIQYKDYAVWQQQFIQSELYKKQEEHWLKELDG
ELPVLTLPTDYSRPAVQTFEGDRIAFSLEAGKADALRRLAKETDSTLYMVLLASYSAFLSK
ISGQDDIIVGSPVAGRSQADVSRVIGMFVNTLALRTYPKGEKTFADYLNEVKETALSAFDA
QDYPLEDLIGNVQVQRDTSRNPLFDAVFSMQNANIKDLTMKGIQLEPHPFERKTAKFDLT
LTADETDGGLTFVLEYNTALFKQETIERWKQYWMELLDAVTGNPNQPLSSLSLV

SEQ ID NO: 70

LTPMQEGMLYHAMLDPHSSSYFTQLELGIHGAFDLEIFEKSVNELIRSYDILRTVFVHQQL
QKPRQVVLAERKTKVHYEDISHADENRQKEHIERYKQDVQRQGFNLAKDILFKVAVFRL
AADQLYLVWSNHHIMMDGWSMGVLMKSLFQNYEALRAGRTPANGQGKPYSDYIKWLG
KQDNEEAESYWSERLAGFEQPSVLPGRLPVKKDEYVNKEYSFTWDETLVARIQQTANLH
QVTGPNLFQAVWGIVLSKYNFTDDVIFGTVVSGRPSEINGIETMAGLFINTIPVRVKVERD
AAFADIFTAVQQHAVEAERYDYVPLYEIQKRSALDGNLLNHLVAFENYPLDQELENGSM
EDRLGFSIKVESAFEQTSFDFNLIVYPGKTWTVKIKYNGAAFDSAFIERTAEHLTRMMEAA
VDQPAAFVREYGLV

SEQ ID NO: 71

VSPAQRRMYILNQLGQANTSYNVPAVLLLEGEVDKDRLENAIQQLINRHEILRTSFDMIDG
EVVQTVHKNISFQLEAAKGREEDAEEIIKAFVQPFELNRAPLVRSKLVQLEEKRHLLLIDM
HHIITDGSSTGILIGDLAKIYQGADLELPQIHYKDYAVWHKEQTNYQKDEEYWLDVFKGE
LPILDLPADFERPAERSFAGERVMFGLDKQITAQIKSLMAETDTTMYMFLLAAFNVLLSKY
ASQDDIIVGSPTAGRTHPDLQGVPGMFVNTVALRTAPAGDKTFAQFLEEVKTASLQAFEH
QSYPLEELIEKLPLTRDTSRSPLFSVMFNMQNMEIPSLRLGDLKISSYSMLHHVAKFDLSLE
AVEREEDIGLSFDYATALFKDETIRRWSRHFVNIIKAAAANPNVRLSDVDLL

SEQ ID NO: 72

LSSAQKRMYVLNQLDRQTISYNMPSVLLMEGELDISRLRDSLNQLVNRHESLRTSFMEAN
GEPVQRIIEKAEVDLHVFEAKEDEADQKIKEFIRPFDLNDAPLIRAALLRIEAKKHLLLLDM
HHIIADGVSRGIFVKELALLYKGEQLPEPTLHYKDFAVWQNEAEQKERMKEHEAYWMSV
LSGELPELDLPLDYARPPVQSFKGDTIRFRTGSETAKAVEKLLAETGTTLHMVLHAVFHVF
LSKISGQRDIVIGSVTAGRTNADVQDMPGMFVNTLALRMEAKEQQTFAELLELAKQTNLS
ALEHQEYPFEDLVNQLDLPRDMSRNPLFNVMVTTENPDKEQLTLQNLSISPYEAHQGTSK
FDLTLGGFTDENGIGLQLEYATDLFAKETAEKWSEYVLRLLKAVADNPNQPLSSLLL

SEQ ID NO: 73

LSPMQEGMLFHAILNPGQSFYLEQITMKVKGSLNIKCLEESMNVIMDRYDVFRTVFIHEKV
KRPVQVVLKKRQFHIEEIDLTHLTGSEQTAKINEYKEQDKIRGFDLTRDIPMRAAIFKKAEE
SFEWVWSYHHIILDGWCFGIVVQDLFKVYNALREQKPYSLPPVKPYKDYIKWLEKQDKQ
ASLRYWREYLEGFEGQTTFAEQRKKQKDGYEPKELLFSLSEAETKAETELAKSQHTTLST
ALQAVWSVLISRYQQSGDLAFGTVVSGRPAEIKGVEHMVGLFINVVPRRVKLSEGITFNG
LLKRLQEQSLQSEPHQYVPLYDIQSQADQPKLIDHIIVFENYPLQDAKNEESSENGFDMVD
VHVFEKSNYDLNLMASPGDEMLIKLAYNENVFDEAFILRLKSQLLTAIQQLIQNPDQPVST
INL

SEQ ID NO: 74

SEVQKGLWTLQKMSPEKSAYHVPLCFKFSSGLHHETFQQAFGLVLNQHPILKHVIQEKDG
VPFLKNEPALSIEIKTENISSLKESDIPAFLRKKVKEPYVKENSPLVRVMSFSRSEQEHFLLV
VIHHLIFDGVSSVTFIRSLFDTYQLLLKGQQPEKAVSPAIYHDFAAWEKNMLAGKDGVKH
RTYWQKQLSGTLPNLQLPNVSASSVDSQFREDTYTRRLSSGFMNQVRTFAKEHSVNVTTV
FLSCYMMLLGRYTGQKEQIVGMPAMVRPEERFDDAIGHFLNMLPIRSELNPADTFSSFISK
LQLTILDGLDHAAYPFPKMVRDLNIPRSQAGSPVFQTAFFYQNFLQSGSYQSLLSRYADFF
SVDFVEYIHQEGEYELVFELWETEEKMELNIKYNTGLFDAASISAMFDHFVYVTEQAMLN
PSQPLKEY

SEQ ID NO: 75

LTGAQTGIWFAQQLDPDNPIYNTAEYIEINGPVNIALFEEALRHVIKEAESLHVRFGENMD
GPWQMINPSPDVQLHVIDVSSEPDPEKTALNWMKADLAKPVDLGYAPLFNEALFIAGPDR
FFWYQRIHHIAIDGFGFSLIAQRVASTYTALIKGQTAKSRSFGSLQAILEEDTDYRGSEQYE
KDRQFWLDRFADAPEVVSLADRAPRTSNSFLRHTAYLPPSDVNALKEAARYFSGSWHEV
MIAVSAVYVHRMTGSEDVVLGLPMMGRIGSASLNVPAMVMNLLPLRLTVSSSMSFSELIQ
QISREIRSIRRHHKYRHEELRRDLKLIGENHRLFGPQINLMPFDYGLDFAGVRGTTHNLSAG
PVDDLSINVYDRTDGSGLRIDVDANPEVYSESDIKLHQQRILQLLQTASAGEDMLIGQMELL

SEQ ID NO: 76

LSFAQRRLWFLHCLEGPSPTYNIPVAVRLSGELDQGLLKAALYDLVCRHESLRTIFPESQG
TSYQHILDADRACPELHVTEIAEKELSDRLAEAVRYSFDLAAEPAFRAELFVIGPDEYVLLL
LVHHIVGDGWSLTPLTRDLGTAYAARCHGRSPEWAPLAVQYADYALWQQELLGNEDDP
NSLIAGQLAFWKETLKNLPDQLELPTDYSRPAEPSHDGDTIHFRIEPEFHKRLQELARANR
VSLFMVLQSGLAALLTRLGAGTDIPIGSPIAGRNDDALGDLVGLFINTLVLRTDTSGDPSFR
ELLDRVREVNLAAYDNQDLPFERLVEVLNPARSRATHPLFQIMLAFQNTPDAELHLPDME
SSLRINSVGSAKFDLTLEISEDRLADGTPNGMEGLLEYSTDLFKRETAQALADRLMRLLEA
AESDPDEQIGNLDILAPEEHS

SEQ ID NO: 77

DTVFTIWQEVLKTSDIEWDDGFFDVGGDSLLAVTVADRIKHELSCEFSVTDLFEYSTIKNIS
QYI

SEQ ID NO: 78

EILCDLFMEVLHLPRVGIDDRFFDLGGHSLLAVQLMSRIREALGVELSIGNLFEAPTVAGL
AERL

SEQ ID NO: 79

EILCDLFAEVLGLARVGIDDSFFELGGHSLLAARLMSRIREVMGAELGIAKLFDEPTVAGL
AAHL

SEQ ID NO: 80

ETIAQIWSEVLGRKQIGIHDDFFALGGHSLKAMTAASRIKKELGIDLPVKLLFEAPTIAGISA
YL

SEQ ID NO: 81

ELLAGIWQDVLGMSEVGVTDNFFSLGGDSIKGIQMASRLNQHGWKLEMKDLFQHPTIEEL
TQYV

SEQ ID NO: 82

ESLCRIWQKTLGIEAIGIDDNFFDLGGHSLKGMMLIANIQAELEKSVPLKALFEQPTVRQLA
AY

SEQ ID NO: 83

KKLAEIWEGILGVKAGVTDNFFMIGGHSLKAMMMTAKIQEHFHKEVPIKVLFEKPTIQEL
ALYL

SEQ ID NO: 84

ELLAGIWQDVLGMSEVGVTDNFFSLGGDSIKGIQMASRLNQHGWKLEMKDLFQHPTIEEL
TQYV

SEQ ID NO: 85

QKVADIWAQVLQAEQVGAYDHFFDIGGHSLAGMKMLALVHQELGVELSLKDLFQSPTV
EGLAQVI

SEQ ID NO: 86

KAIAAIWQDVLNVEKAGIFDNFFETGGHSLKAMTLLTKIHKETGIEIPLQFLFEHPTITALAE

SEQ ID NO: 87

LTPIQRWFFEKNFTNKHHWNQSVMLHAKKGFDPERVEKTLQALIEHHDALRMVYREGQE
DVIQYNRGLEAASAQLEVIQIEGQAADYEDRIEREAERLQSSIDLQEGGLLKAGLFQAEDG
DHLLLAIHHLVVDGVSWRILLEDFAAVYTQLEQGNEPVLPQKTHSFAEYAERLQDFANSK
AFLKEKEYWRQLEEQAVAAKLPKDRESGDQRMKHTKTIEFSLTAEETEQLTTKVHEAYH
TEMNDILLTAFGLAMKEWTGQDRVSVHLEGHGREEIIEDLTISRTVGWFTSMYPMVLDM
KHADDLGYQLKQMKEDIRHVPNKGVGYGILRYLTAPEHKEDVAFSIQPDVSFNYLGQFD
EMSDAGLFTRSELPSGQSLSPETEKPNALDVVGYIENGKLTMSLAYHSLEFHEKTVQTFSD
SFKAHLLRIIEHCLSQDGTELTPSDLG

SEQ ID NO: 88

PPLFCVHPAGGLSWCYAGLMTNIGTDYPIYGLQARGIGQREELPKTLDDMAADYIKQIRT
VQPKGPYHLLGWSLGGNVVQAMATQLQNQGEEVSLLVMLDAYPNHFLPIKEAPDDEEA
LIALLALGGYDPDSLGEKPLDFEAAIEILRRDGSALASLDETVILNLKNTYVNSVGILGSYK
PKTFRGNVLFFRSTIIPEWFDPIEPDSWKPYINGQIEQIDIDCRHKDLCQPEP

SEQ ID NO: 89

DILQVGMIWK
adenylation domain of B. amyloliquefaciens

SEQ ID NO: 90

EEHMILKTWNETSREYPDACFHELFERQAAETPDACAVVYEQQQLTYRELDEKSTKLALY
LQAHGAGPDDLIGIYTDRSLHMAVGLLGILKAGGAYVPLDPSYPADRLEYMIADSRISMC
LTTADLEHSLNWGGVQTTAIDRDWSHIESTAAERTSLKRLVTPDDLAYVIYTSGSTGKPK
GVMIPHRALTNFLISMANEPGLSSEDKLLAVTTYCFDIAALELYLPLIKGAECNICKTEVTK
DARKLKELIQEYKPTIMQATPFTWKMLFHSGWSNEENVKILCGGEALSEQLKQQFLDTKS
EAWNMFGPTETTIWSAVQRITENESALTIGRPIANTRVYIMDSGLNPVLEGVPGELCIAGD
GLARGYFNKPELTDKAFVSHRLELGSKLYKTGDMARFLPGGRIEYMGRMDTQVKIRGYR
IEPGDIESRLNAHPAVQESVVVVNNHSGNEKLCAFYIRKNGEPLPSAKELRKHMKQALPA
YMAPASFVRLEELPLTPNGKVDRKLLAARDLTEKQPDT
B. amyloliquefaciens

SEQ ID NO: 91

MKSDMKASGLLKPSVSYGKSLRLSADQPMTIPEVLHKTAAAAGDQKGITYIQPDGTKVY
QTYSSLKKIALSIVKGLRQSGVKAQDEVILQLSDNSQLIPAFWGCVFLGAIPVPLAAAPAYT
EMNSGTQKLKDAWTLLNQPYVITSRDVLPEMTEWAEEQGLSGFCALAAEDLSAHEMDT
DCHHPRPEDLAMLLLTSGSTGTPKAVMLSHENIVCMVKGNIQMQGYTSEDVTFNWMPFD
HVGGIGMLHLRDVYLGCEEINIPSESILMDPLKWLDLIDHYRASVTWAPNFAFGLLADFAE
EIQTRKWDLSSMRYMLNGGEAAVAKVGRRIMELLEPHGLPANAIRPAWGMSETSSGVIFS
DEFTLENTNDDDRFVEIGLPIPGFNMRITDDRNQVVEEGEIGRFQVSGLTVTSGYYERPDL
NESVFTEDGWFETGDLGFLREGRLTITGRTKDAIIINGVNYYSHAIESAVEELPEIETSYTAA
CAVRPNQSTTDELAIFFVTSVPLDENRMTKLLHHIHQHVTQRIGVTPDYLLPVAKEDIPKT
AIGKIQRTQLKHSFEQGQFDSLHNNRQEGGNSDTASAEKEIERDFIRFLKEELSIAEELVDP
NTPFQSLGVNSIKMMKLARSIEKTYHIRLTARELHKNPTIGALAAYTAEKAANMSAEHHP
QKAEPPAERERRQTAPALSEVQKGLWTLQKMSPETTAYHVSLCFRFTSGIDKEKLKQAFQ
LVLKQHPMLKSAVNETDGGFYFIEAADPFVFSEEDISDVNETQISALIRKKVKEPFVKEGGP
LLRVQLLSKSAEEHYLLTVIHHIVFDGISSITFIHSLLDHYQALLEGKEEFTAAAPGIHPDFA
AWEKRYLAGDESKAARAYWMKQLEGDLPDLQLPPMRTDSSVPEFTEDTLTRRLPERLLN
NITAFAASRSIHLSTVMLSCYMVLLSKYTDKEDIVTGMPAMVRPEERFDGVVGHFLNMLP
IRSRAGRKETFADFVQKVQDAVLDGLDHSFYPFPKMVRDVHAKPLQNGSPVFSNAFFYQ
NFLQHSSYQSMLDEYKEFSCEFVKDIHQEGEYDLVFEMWEEASGMDLTIKYSTELFDEAG
AARLFSQFVQAAEALTANPDTPLENVSLMTKREEHMILKTWNETSREYPDACFHELFERQ
AAETPDACAVVYEQQQLTYRELDEKSTKLALYLQAHGAGPDDLIGIYTDRSLHMAVGLL
GILKAGGAYVPLDPSYPADRLEYMIADSRISMCLTTADLEHSLNWGGVQTTAIDRDWSHI
ESTAAERTSLKRLVTPDDLAYVIYTSGSTGKPKGVMIPHRALTNFLISMANEPGLSSEDKL
LAVTTYCFDIAALELYLPLIKGAECNICKTEVTKDARKLKELIQEYKPTIMQATPFTWKML
FHSGWSNEENVKILCGGEALSEQLKQQFLDTKSEAWNMFGPTETTIWSAVQRITENESAL
TIGRPIANTRVYIMDSGLNPVLEGVPGELCIAGDGLARGYFNKPELTDKAFVSHRLELGSK
LYKTGDMARFLPGGRIEYMGRMDTQVKIRGYRIEPGDIESRLNAHPAVQESVVVVNNHS
GNEKLCAFYIRKNGEPLPSAKELRKHMKQALPAYMAPASFVRLEELPLTPNGKVDRKLLA
ARDLTEKQPDTQTFSSSHIQQAVLAIWQDVLKSKDIELDDRFFDAGGDSLLAVTVTDRMT
QELDCEFSVTELFEYATVKDISRYITEQKPKEAFLAPVPQKKVNAEDREHPAGEFPDYYED
SLAVIGISCEFPGAKDHYEFWNNIKEGKESITFFSKEELRRSGISEELADHPGFVPAKSVLEG
KEMFDPGFFGFSPKDAEYMDPQLRMLLLHSWKAIEDAGYISKEIPETSVYMSASTNSYRSL
LPEETTAQLETPDGYVSWVLAQSGTIPTMISHKLGLKGPSYFVHANCSSSLIGLHSAFQSLQ
SGEAKYALVGGATLHTESSAGYVHQPGLNFSSDGHIKAFDADADGMIGGEGAGAVLLKK
ASDAVKDGDHIYALLRGIGVNNDGADKVGFYAPSVKGQAEVIQKVIDQTGIHPETIAYVE
AHGTGTKLGDPIELSALQSVYGRYTDKKQYCGIGSVKTNLGHLDTAAGMAGCIKVVMSL
YHQEIAPSINYKEPNPNLHLEDSPFFVAEEKKELTRENRAHRMALSSFGLGGTNTHAIFEQ
YPDASEAADAAGPFIIPLSARKKDRLKEYAKQLLAFLERKTDTDLADLAYTFQVGREAME
ERAAFITSGTAELKRQLADFINDKPAVTGCFRGEKQQAKDIAWLSDDDDSAELIEKWLAK
GKGPKLCEMWSKGVAINWHKLYKDKHPKRISLPVYPFAKEPYWPKKAEKNTSAAHTGV
SVLHPLVQQNTSDLAVQRFSSRFTGSEFFLKDHVVREKAVLPGAAYLEMSYEAVKRALG
GLLKDQDRITLHHTIWMKPIVVHEQERQVHIALFPEEDGIISYDIYSVNADGEEVLHSQGR
AEIIKAEREPEADLSAIQNRCTADILDPAAFYREGRSRGMFHGKAFQGIKSAFIGEKEVLSDI
QLPDSVSHTNGQFTLHPSIIDSAIQTATICIMQEFSGQKLILPFALEYLEVIKACTPTMRAHA
RFSDGYQTGNSVQKADIDLFDETGALCVKVRGFSTRVLEGDAESAQPPSGSEKVLFAPVW
KEAAASEKASVRADEHLIILCEDACRIKDETASGLKDAELLIAEGEGETAAERFQSYAQTIA
EHIRRIISEKRQGRILIQTVISADGSQQLFAGLTGLLKTAELEYGKLDCQLIEMESIGEMPAV
AQKLKNDSHQPHDKHIRYKGEKRYVKAWEEMHPAGGGIVWKDEGVYLITGGAGSLGLM
VAKEIAGRVKHPKIILTGRSNLTEAKQRELAMLQHSGANVVYKKLNAADRRENEAMIKEI
TAEFGKLNGIIHAAGVIQDRFLLQKTKEEFREVLDPKVIGTAYLDEASKDCNLDFFVLFSSV
SGVLGNVGQADYAAGNAFMDAYAAYRQSLTENGLRHGKTVAFNWTLWKDGGMQAGD
ETERMMKKAFGMVPLQKASGLAAFYEGMAQEKPQLLAAEGHAAKLKQSFLSVSETEKP
QVKKIEPVSAVSGDKWHGALIRLVSSILKVGQDEIDIDTELSEYGFDSVSFTVFTNQLNEAY
QLKLAPTIFFEHGTIRSLADYLTDEAEAGLPSQPEEKHDADKSLQTLHTALTAMVSGILKV
DREDIETDTELSEYGFDSVSFTVFTNQLNEAYQLELAPTIFFEHGTISGLAGYLAKEHPGRF
GEKKKESPKKEQPKAQKPKMQRKKRFATVMNASAATQEPRRFDPVAIVGISGRFPGAKDI
EEFWRNLKEGKDSITTIPKERWDWQAFDGDPNLEGNKTNIKWGGFIDGIAEFDPLFFGISP
REAQYLDPQQRLLLTYAWRAIEDAGCKPESLSGTNTGVFIGTGNTGYKDLFTRAGLAPEG
HAATGSMIPSIGPNRLSYLLNLHGPSEPIETACSSSLVAIHRAVSAIENGECDMAIAGGINTIL
TEEAHISYSKAGMLSKDGKCKTFSKDANGYVRGEGAGILMLKKLSDAERDGNPIYGVIRG
TAENHGGRANTLTSPNPKLQADLLVKAYRKAGVDPSTVTYIEAHGTGTELGDPIEINGLK
AAFHELAKTNQEPEVSGHRCGIGSVKSNIGHLELAAGVSGVMKVLLQMKHKTLVKSLHC
ETINPYIQLDDSPFYIVRENQEWTAAKDRNGNAIPRRAGVSSFGIGGVNAHIVIEEYVPKAA
AQPEHSPENPAIILLSAKSKEKLFEQAQQLQKAIRQTPYTDQDLAGVAYTLQTGRDEMEER
LAILAATMAELETKLEAFTKNEKNIAGLYTGQSHRNKDTFALFTADEDMDIVIDAWIKKG
KFAKLAEVWVKGGVFNWNRLYEKGTPRLLSLPSYPFAKDTYWVPENPDKNKVHTEERM
KRILTKQWELSPLELREPCARSVAILTGGDTINLAEEIAAHMPNHRIITASDLSGEYDWQAF
DGVIDLLGAAKEDSQDMAGIKWLQQLIEHGHKEGMTLLCVTKDLESLNKEANQTTGAKR
AGLYRMLAYEYGHLSSRHLDLEGALSDDKLAKQIADEFHARTDDAEVCRRDGLRYRAVL
SGAENMNMKGERIDFPDNHVLLITGGTRGIGLLCARHFAEHYGVKKLVLTGRETLPPRSE
WTGGLNGVPASVKAKIEAVLDLESKGVQVKVLSVPLADEAGLRQELSQIKQTLGPIGGVI
HCAGVTDKETLAFIRKTDEDIQRVLEPKVDGLQALYSVLSEEPLKFFVLFSSVSAAIPALSA
GQADYAMANAYMDYFANSYKEQLPIVSIQWPNWKETGMGEVTNKAYKESGLYSITNAE
GLQLLDGILLESARGPVLPAVLNPKLWKPERFLRRSRQDQPMSPAMVKPAEKTAYTAENN
TQLTRKTEEWLKELFSEELRIDQDQLETDVLFQDYGVDSIILAQLLQRINRNLSASLDPSILY
EYPTIQSFANWLLEGYTEVLSERFDLAAEPVKKNPAEPVIETQAVKVQEEPAGKQIHREDI
AVVGMSCRFPGAASLEAYWSLLAEGRSAIRPVPAERWGLKTPYYAGMLDGIHQFDPDFF
LLAEEDVKAMDPQALAALEECLNLWYHAGYTPDEIKGEAIGVYLGGRSRHRPGEDKLLD
AKNPIVALGQNYLAANLSQYFDMRGPSVVLDTACSSALVGMNMAVQALVSGEIKAAVV
GGVSLFESEETHKLFEQRGILSKAQSFHVFDERADGVVLGEGVGMVLLKTVSQAIEDGDSI
YAVVKAASVNNDGRTAGPATPSLEAQKSVMKTALEKSGKQPEDITHIEANGSGTVVTDL
LELKAIQSVYRSKDAGPLGIGSVKPNIGHPLCAEGIASFIKVVLMLKEKSFIPFLSGEHENTH
FDREKANIQFSRTLADWPSPIPAAGINCFADGGTNAHVIVEAWQEDEGRRIKRHPLTPPAL
NKRLISPDSKEETNEKAAANIWDTYEVEV
S. roseosporous dptA

SEQ ID NO: 92

TDDERDRILGDWGSGTHTPLPPRSVAEQIVRRAALDPDAVAVITAEEELSYRELERLSGE
TARLLADRGIGRESLVAVALPRTAGLVTTLLGVLRTGAAYLPLDTGYPAERLAHVLSDA
RPDLVLTHAGLAGRLPAGLAPTVLVDEPQPPAAAAPAVPTSPSGDHLAYVIHTSGSTGR
PKGVAIAESSLRAFLADAVRRHDLTPHDRLLAVTTVGFDIAGLELFAPLLAGAAIVLADE
DAVRDPASITSLCARHHVTVVQATPSWWRAMLDGAPADAAARLEHVRILVGGEPLPAD
LARVLTATGAAVTNVYGPTEATIWATAAPLTAGDDRTPGIGTPLDNWRVHILDAALGP
VPPGVPGEIHIAGSGLARGYLRRPDLTAERFVANPFAPGERMYRTGDLGRFRPDGTLEH
LGRVDDQVKVRGFRIELGDVEAALARHPDVGRAAAAVRPDHRGQGRLVAYVVPRPGT
RGPDAGELRETVRELLPDYMVPSAQVTLTTLPHTPNGKLDRAALPAPVFGTPAGR
adenylation domain of S. roseosporous

SEQ ID NO: 93

MDMQSQRLGVTAAQQSVWLAGQLADDHRLYHCAAYLSLTGSIDPRTLGTAVRRTLDET
EALRTRFVPQDGELLQILEPGAGQLLLEADFSGDPDPERAAHDWMHAALAAPVRLDRAG
TATHALLTLGPSRHLLYFGYHHIALDGYGALLHLRRLAHVYTALSNGDDPGPCPFGPLAG
VLTEEAAYRDSDNHRRDGEFWTRSLAGADEAPGLSEREAGALAVPLRRTVELSGERTEKL
AASAAATGARWSSLLVAATAAFVRRHAAADDTVIGLPVTARLTGPALRTPCMLANDVPL
RLDARLDAPFAALLADTTRAVGTLARHQRFRGEELHRNLGGVGRTAGLARVTVNVLAY
VDNIRFGDCRAVVHELSSGPVRDFHINSYGTPGTPDGVQLVFSGNPALYTATDLADHQER
FLRFLDAVTADPDLPTGRHRLLSPGTRARLLDDSRGTERPVPRATLPELFAEQARRTPDAP
AVQHDGTVLTYRDLHRSVERAAGRLAGLGLRTEDVVALALPKSAESVAILLGIQRAGAA
YVPLDPTHPAERLARVLDDTRPRYLVTTGHIDGLSHPTPQLAAADLLREGGPEPAPGRPAP
GNAAYIIQTSGSTGRPKGVVVTHEGLATLAADQIRRYRTGPDARVLQFISPGFDVFVSELS
MTLLSGGCLVIPPDGLTGRHLADFLAAEAVTTTSLTPGALATMPATDLPHLRTLIVGGEVC
PPEIFDQWGRGRDIVNAYGPTETTVEATAWHRDGATHGPVPLGRPTLNRRGYVLDPALEP
VPDGTTGELYLAGEGLARGYVAAPGPTAERFVADPFGPPGSRMYRTGDLVRRRSGGMLE
FVGRADGQVKLRGFRIELGEVQAALTALPGVRQAGVLIREDRPGDPRLVGYIVPAPGAEP
DAGELRAALARTLPPHMVPWALVPLPALPLTSNGKLDRAALPVPAARAGGSGQRPVTPQ
EKTLCALFADVLGVTEVATDDVFFELGGHSLNGTRLLARIRTEFGTDLTLRDLFAFPTVAG
LLPLLDDNGRQHTTPPLPPRPERLPLSHAQQRLWFLDQVEGPSPAYNIPTAVRLEGPLDIPA
LAVALQDVTNRHEPLRTLLAEDSEGPHQVILPPEAARPELTHSTVAPGDLAAALAEAARR
PFDLAGEIPLKAHLFGCGPDDHTLLLLVHHTAGDGASVEVLVRDLAHAYGARRAGDAPH
FEPLPLQYADHTLRRRHLLDDPSDSTQLDHWRDALAGLPEQLELPTDHTRPAVPTRRGEAI
AFTVPEHTHHTLRAMAQAHGVTVFMVMQAALAALLSRHGAGHDIPLGTPVAGRSDDGT
EDLVGFFVNTLVLRNDVSGDPTFAELVSRVRAANLDAYAYQDVPFERLVDVLKPERSLS
WHPLFQIMIAYNGPATNDTADGSRFAGLTSRVHAVHTGMSKFDLSFFLTEHADGLGIDGA
LEFSTDLFTRITAERLVQRYLTVLEQAAGAPDRPISSYELLGDDERALLAQWNDTAHPTPP
GTVLDLLESRAARTPDRPAVVENDHVLTYADLHTRANRLARHLITAHGVGPERLVAVAL
PRSAELLVALLAVLKTGAAYVPLDLTHPAERTAVVLDDCRPAVILTDAGAARELPRRDIP
QLRLDEPEVHAAIAEQPGGPVTDRDRTCVTPVSGEHVAYVIYTSGSTGRPKGVAVEHRSL
ADFVRYSVTAYPGAFDVTLLHSPVTFDLTVTSLFPPLVVGGAIHVADLTEACPPSLAAAGG
PTFVKATPSHLPLLTHEATWAASAKVLLVGGEQLLGRELDKWRAGSPEAVVFNDYGPTE
ATVNCVDFRIDPGQPIGAGPVAIGRPLRNTRVFVLDGGLRAVPVGVVGELHVAGEGLARG
YLGQPGLTAERFVACPFGDAGERMYRTGDLVRWRADGMLEFVGRVDDQVKVRGFRIEL
GEVEAAVAACPGVDRSVVVVREDRPGDRRLVAYVTAAGDEAEGLAPLIVETAAGRLPGY
MVPSAVVVLDEIPLTPNGKVDRAALPAPRVAPAAEFRVTGSPREEALCALFAEVLGVERV
GVDDGFFDLGGDSILSIQLVARARRAGLEVSVRDVFEHRTVRALAGVVRESGGVAAAVV
DSGVGAVERWPVVEWLAERGGGGLGGAVRAFNQSVVVATPAGITWDELRTVLDAVRER
HDAWRLRVVDSGDGAWSLRVDAPAPGGEPDWITRHGMASADLEEQVNAVRAAAVEAR
SRLDPLTGRMVRAVWLDRGPDRRGVLVLVAHHLVVDGVSWRIVLGDLGEAWTQARAG
GHVRLDTVGTSLRGWAAALAEQGRHGARATEANLWAQMVHGSDPLVGPRAVDPSVDV
FGVVESVGSRASVGVSRALLTEVPSVLGVGVQEVLLAAFGLAVTRWRGRGGSVVVDVEG
HGRNEDAVPGADLSRTVGWFTSIYPVRLPLEPAAWDEIRAGGPAVGRTVREIKECLRTLP
DQGLGYGILRYLDPENGPALAQHPTPHFGFNYLGRVSVSADAASLDEGDAHADGLGGLV
GGRAAADSDEEQWADWVPVSGPFAVGAGQDPVLPVAHAVEFNAITLDTPDGPRLSVTW
SWPTTLLSESRIRELARFWDEALEGLVAHARRPDAGGLTPSDLPLVALDHAELEALQADV
TGGVHDILPVSPLQEGLLFHSSFAADGVDVYVGQLTFDLTGPVDADHLHAVVESLVTRHD
VLRTGYRQAQSGEWIAVVARQVHTPWQYIHTLDTDADTLTNDERWRPFDMTQGPLARF
TLARINDTHFRFIVTYHHVILDGWSVAVLIRELFTTYRDTALGRRPEVPYSPPRRDFMAWL
AERDQTAAGQAWRSALAGLAEPTVLALGTEGSGVIPEVLEEEISEELTSELVAWARGRGV
TVASVVQAAWALVLGRLVGRDDVVFGLTVSGRPAEVAGVEDMVGLFVNTIPLRARMDP
AESLGAFVERLQREQTELLEHQHVRLAEVQRWAGHKELFDVGMVFENYPMDSLLQDSLF
HGSGLQIDGIQGADATHFALNLAVVPLPAMRFRLGYRPDVFDAGRVRELWGWIVRALEC
VVCERDVPVSGVDVLGAGERETLLGWGAGAEPGVRALPGAGAGAGAGLVGLFEERVRT
DPDAVAVRGAGVEWSYAELNARANAVARWLIGRGVGPERGVGVVMDRGPDVVAMLL
AVAKSGGFYLPVDPQWPTERIDWVLADAGIDLAVVGENLAAAVEAVRDCEVVDYAQIA
RETRLNEQAATDAGDVTDGERVSALLSGHPLYVIYTSGSTGLPKGVVVTHASVGAYLRR
GRNAYRGAADGLGHVHSSLAFDLTVTVLFTPLVSGGCVTLGDLDDTANGLGATFLKATP
SHLPLLGQLDRVLAPDATLLLGGEALTAGALHHWRTHHPHTTVINAYGPTELTVNCAEY
RIPPGHCLPDGPVPIGRPFTGHHLFVLDPALRLTPPDTIGELYVAGDGLARGYLGRPDLTAE
RFVACPFRSPGERMYRTGDLARWRSDGTLEFIGRADDQVKIRGFRIELGEVEAAVAAHPH
VARAIAVVREDRPGDQRLVAYVTGSDPSGLSSAVTDTVAGRLPAYMVPSAVVVLDQIPLT
PNGKVDRAALPAPGTASGTTSRAPGTAREEILCTLFADVLGLDQVGVDEDFFDLGGHSLL
ATRLTSRIRSALGIDLGVRALFKAPTVGRLDQLLQQQTTSLRAPLVARERTGCEPLSFAQQ
RLWFLHQLEGPNAAYNIPMALRLTGRLDLTALEAALTDVIARHESLRTVIAQDDSGGVW
QNILPTDDTRTHLTLDTMPVDAHTLQNRVDEAARHPFDLTTEIPLRATVFRVTDDEHVLLL
VLHHIAGDGWSMAPLAHDLSAAYTVRLEHHAPQLPALAVQYADYAAWQRDVLGTENN
TSSQLSTQLDYWYSKLEGLPAELTLPTSRVRPAVASHACDRVEFTVPHDVHQGLTALART
QGATVFMVVQAALAALLSRLGAGTDIPIGTPIAGRTDQAMENLIGLFVNTLVLRTDVSGD
PTFAELLARVRTTALDAYAHQDIPFERLVEAINPERSLTRHPLFQVMLAFNNTDRRSALDA
LDAMPGLHARPADVLAVTSPYDLAFSFVETPGSTEMPGILDYATDLFDRSTAEAMTERLV
RLLAEIARRPELSVGDIGILSADEVKALSPEAPPAAEELHTSTLPELFEEQVAARGHAVAVV
CEGEELSYKELNARANRLARVLMERGAGPERFVGVALPRGLDLIVALLAVTKTGAAYVP
LDPEYPTDRLAYMVTDANPTAVVTSTDVHIPLIAPRIELDDEAIRTELAAAPDTAPCVGSGP
AHPAYVIYTSGSTGRPKGVVISHANVVRLFTACSDSFDFGPDHVWTLFHSYAFDFSVWEI
WGALLHGGRLVVVPFEVTRSPAEFLALLAEQQVTLLSQTPSAFHQLTEAARQEPARCAGL
ALRHVVFGGEALDPSRLRDWFDLPLGSRPTLVNMYGITETTVHVTVLPLEDRATSLSGSPI
GRPLADLQVYVLDERLRPVPPGTVGEMYVAGAGLARGYLGRPALTAERFVADPNSRSGG
RLYRTGDLAKVRPDGGLEYVGRGDRQVKIRGFRIELGEIEAALVTHAGVVQAVVLVRDE
QTDDQRLVAHVVPALPHRAPTLAELHEHLAATLPAYMVPSAYRTLDELPLTANGKLDRA
ALAGQWQGGTRTRRLPRTPQEEILCELFADVLRLPAAGADDDFFALGGHSLLATRLLSAV
RGTLGVELGIRDLFAAPTPAGLATVLAASGTALPPVTRIDRRPERLPLSFAQRRLWFLSKLE
GPSATYNIPVAVRLTGALDVPALRAALGDVTARHESLRTVFPDDGGEPRQLVLPHAEPPFL
THEVTVGEVAEQAASATGYAFDITSDTPLRATLLRVSPEEHVLVVVIHHIAGDGWSMGPL
VRDLVTAYRARTRGDAPEYTPLPVQYADYALWQHAVAGDEDAPDGRTARRLGYWREM
LAGLPEEHTLPADRPRPVRSSHRGGRVRFELPAGVHRSLLAVARDRRATLFMVVQAALA
GLLSRLGAGDDIPIGTPVAGRGDEALDDVVGFFVNTLVLRTNLAGDPSFADLVDRVRTAD
LDAFAHQDVPFERLVEALAPRRSLARHPLFQIWYTLTNADQDITGQALNALPGLTGDEYP
LGASAAKFDLSFTFTEHRTPDGDAAGLSVLLDYSSDLYDHGTAAALGHRLTGFFAALAAD
PTAPLGTVPLLTDDERDRILGDWGSGTHTPLPPRSVAEQIVRRAALDPDAVAVITAEEELS
YRELERLSGETARLLADRGIGRESLVAVALPRTAGLVTTLLGVLRTGAAYLPLDTGYPAE
RLAHVLSDARPDLVLTHAGLAGRLPAGLAPTVLVDEPQPPAAAAPAVPTSPSGDHLAYVI
HTSGSTGRPKGVAIAESSLRAFLADAVRRHDLTPHDRLLAVTTVGFDIAGLELFAPLLAGA
AIVLADEDAVRDPASITSLCARHHVTVVQATPSWWRAMLDGAPADAAARLEHVRILVGG
EPLPADLARVLTATGAAVTNVYGPTEATIWATAAPLTAGDDRTPGIGTPLDNWRVHILDA
ALGPVPPGVPGEIHIAGSGLARGYLRRPDLTAERFVANPFAPGERMYRTGDLGRFRPDGTL
EHLGRVDDQVKVRGFRIELGDVEAALARHPDVGRAAAAVRPDHRGQGRLVAYVVPRPG
TRGPDAGELRETVRELLPDYMVPSAQVTLTTLPHTPNGKLDRAALPAPVFGTPAGRAPAT
REEKILAGLFADILGLPDVGADSGFFDLGGDSVLSIQLVSRARREGLHITVRDVFEHGTVG
ALAAAALPAPADDADDTVPGTDVLPSISDDEFEEFELELGLEGEEEQW
S. roseosporous dptBC

SEQ ID NO: 94

AGERDQLLHEWNDTAAALPPALLPQLFEEQVRRTPHDVALVSGNIRLTYAELDARANR
LAHLLLARGAAPETFVAVALPRTEELLVALLAVQKTGAGHLPLDPGFPAERLSYMLDD
ARPAVVLTTEDISARIPGGSHVVLDSEQVTGELHDHPATSPAGRGNPAGPAYVIYTSGST
GQPKGVVVPSAALVNFLADMVPRLGLRGGDRLLSVTTVGFDIAALELFVPLLSGATVVL
ADGETVRDPALARQTCEDHGVTMVQATPSWWHGMLADAGDSLRGVHAVVGGEALSP
GLRDALTRGARSVTNMYGPTETTIWSTSAGQAAGDSAPPSIGTPILNTRVYVLDAALCV
VPPGVAGELYIAGDGLARGYLGRAGLTAERFVACPFGAPGERMYRTGDLVRWRVDGA
LEFVGRADDQVKVRGFRVELGEVEGAVAAHPDVVRAVVVVREDRPGDHRLVAYVTG
VDTGGLSSAVMRAVAERLPAYMVPSAVVVLDEIPLTPNGKVDRAALPVPGVEAGAGYR
adenylation domain of S. roseosporous

SEQ ID NO: 95

MNRRSKVVEEILPVSALQEGLLFHSSFAAADGVDVYAGQLAFDLVGAVDTGRLRAAVES
LVARHGVLRSSYRQARSGEWVAVVARRVATPWRAVDARDGATDAAAVAREERWRPFD
LGRAPLARFVLVRTDDDRFRFVITYHHVILDGWSLPVLLRELLALYGSGADPSVLPPVRPY
GDFLRWAAARDDAAAETAWRDALTGLDEPSLVAPGASPDGVVPASVHAELDKAGTENL
AAWARHRGITQATAVRAAWALVLGQHTGRDDVVFGVTVSGRPAELAGAEHMVGLFINT
VPLRTVLDPADTLGTFAARLQAEQTTLLEHQHVRLSDIQRWAGHKELFDTIVVFENYPIGH
SGPGSIRTDDFTVTATEGSDATHYPLTLTAVPGETLRLKLDHRPDLVDTTTATALLRRVTR
VLETATDDTGHTLARLDLLDDDERHRLLRGWNDTTREQPPTYYHQEFEEQARRRPHDTA
LVFTSTSWTYEELNDRANRLARLLVAAGAGSDDFVALAFPRSAESVVAILAVLKAGAAY
LPLDMDQPAERLTGILADAHPTVVLTTTTATPLPHPGRTLVLDSPTTARALAAAPAHNLTD
ADRRTPLNARNAAYIIHTSGSTGRPKGVVIEHRSLANLFHDHRRALIEPHAAGGSRLKAGL
TASLSFDTSWEGLICLAAGHELHLIDDDTRRDAERVAELIDRQRIDVIDVTPSFAQQLVETG
ILDEGRHHPAAFMLGGEGVDAKLWTRLSDVPGVTSYNYYGPTEFTVDALACTVGIAPRP
VIGHPLDNTAAYILDGFLRPVPEGVAGELYLAGTQLARGYAGRPGLTAERFVACPFGAPG
ERMYRTGDLVRRSPGGVVEYLGRVDDQIKLRGFRIEPAEIELALAGHPAVAQNVVLLHRS
ATGEARLVAYVVPGTPVDPRELTGHLAARLPAYMVPSAFVLLDTLPLTPNGKLDRGALPE
PAFGTAPRPERPRTPVEEILCGLYADVLGLPSFGADDDFFDAGGHSLLASKLVSRIRTNLKT
ELNVRALFEHRTVSSLATALHRAAQAGPALTAGPRPARIPLSYAQRRLWFLNRLDRDSAA
YNMPVALRLRGPLDSTAMCAALTDVAERHEALRTVFEEDRDGAHQIVLPATGLGPLLTV
TGADGTTLRALITEFVRRPFDLAAEIPFRAALFRVGDEEHVLVVVLHHIAGDGWSMGPLA
RDVAEAYRARAAGRAPDWEPLPVQYADYALWQREVLGAEDDETGELSAQLAHWRTRL
AGAPAELTLPTDRPRPAVASTAGDRVEFTVPAGLHQALADLARAHGATVFMVVQAALA
VLLSRLGAGDDIPIGTPVAGRTDEATEELIGFFVNTLVLRTDVSGDPTFAELLARVRATDL
DAYAHQDVPFERLVEVLNPERSLARHPLFQVMLTFNVPDMDGVGSALGNLGELEVSGEA
IRTDQTKVDLAFTCTEMYAADGAASGMRGVLEYRLDVFGAVQARETTERLVRVLEGVVS
GGGGVSVSGVDVLGVGERERLLGWGVGGPVPVVPGGGLVGLFEERVRADADAVAVRG
AGVVWSYGELNARVNVVARWLVGRGVGAECGVGVVMGRGVDVVVMLLAVAKAGGF
YVPVDPEWPVERVGWVLADAGVGLVVVGEGLSHVVGDFPGGEVFEFSRVVRESCLVEL
VAADGVEVRNVTDGERASRLLPGHPLYVVYTSGSTGRPKGVVVTHASVGGYLARGRDV
YAGAVGGVGFVHSSLAFDLTVTVLFTPLVSGGCVVLGELDESAQGVGASFVKVTPSHLGL
LGELEGVVAGNGMLLVGGEALSGGALREWRERNPGVVVVNAYGPTELTVNCAEFLIAPG
EEVPDGPVPIGRPFAGQRMFVLDAALRVVPVGVVGELYVAGVGLARGYLGRAGLTAERF
VACPFGAPGERMYRTGDLVRWRVDGALEFVGRADDQVKVRGFRVELGEVEGAVAAHP
DVVRAVVVVREDRPGDHRLVAYVTGVDTGGLSSAVMRAVAERLPAYMVPSAVVVLDEI
PLTPNGKVDRAALPVPGVEAGAGYRAPVSPREEVLCGLFAEVLGLERVGVDDDFFGLGG
HSLLATRLISRVRAVLGVEAGVRALFEAPTVSRLERLLRERSALGVRVPLVARERTGREPL
SFAQQRLVVFLEELEGPGAAYNIPMALRLAGVLDVEALHQALIDVIARHESLRTLIAQDAG
TAWQHILPVDDPRTRPGLPLVDIGADALQERLDEAAGRPFDLAADLPVRATVFRLTDNDH
ILLVVAHHVAFDAMSRVPFIRNVKRAFEARTNGAAPDWRPLPVQYADYAAWQRDVLGT
EDDESSELSAQLAYWRTQLASLPAELALPTDRARPAVASYEGGKVEFTVPAGVYDGLVA
LARAEGVTVFMVVQAALAALLSRLGAGDDIPIGTPIAGRTDQATEDLIGFFVNTLVLRTDV
SGDPTFAELLARVRATDLDAYAHQDIPFERLVEAVNPERSLARHPLFQVMLTFDNTIDREV
TEGFAGLGVEGLPLGAGAVKFDLLFGLSEVGGELRGAVEYRCDLFDHPTVAQLAERLVR
VLERVASDASVRTGELPVVGEAERARVLTEWNDTGVPGVPETFLELFEAQVAARGDAPA
VVYEGEVLSYRELDARANRLAGLLVGRGAGPEHFVGVALPRGLDLIVALLAVLKSGAAY
VPLDPEYPAERLVHMVTDAAPVVVVTSTDVRTLRTVPRVELDDEATRATLVAAPATGPD
VKMSASHPAYVIYTSGSTGRPKGVVISHGSLANFLAWAREDLGAERLRHVVLSTSLSFDV
SVVELFAPLSCGGTVEIVRNLLALVDRPGRWSASLVSGVPSAFAQLLEAGLDRADVGMIA
LAGEALSARDVRRVRAVLPGARVANFYGPTEATVYATAWYGDTPMDAAAPMGRPLRNT
CVYVLDDGLRVVPVGVVGELYVAGVGLARGYLGRVGLTAERFVACPFGARGERMYRTG
DLVRWRVDGTLEFVGRADDQVKVRGFRVELGEVEGAVAAHPDVVRAVVVVREDRPGD
HRLVAYVTGVDTGGLSSAVMRAVAERLPAYMVPSAVVVLDEIPLTPNGKVDRAGLPVPV
VSVAGFCAPSSPREEVLCGLFAEVLGVERVGVDDGFFDLGGDSILSIQLVARARRAGLELS
VRDVFEGRTVRALAAVVRGSDAGAVGVVGGAEIVLPGVGEVERWPVVEWLAERGGGSL
GGVVRGFNQSVVLAVPAGLVWEELRVLLGAVRDRHEAWRLRVLDSGALCVDGVVPDD
GSWIVRCDLSGMGVDGQVDAVRAAAVEARAWLDPSVGRVVRAVWLERGGDRSGVLVL
VAHHLVVDGVSWRVVLGDLAEGWAQVRSGGRVELGVVGTSLRGWAAALAEQGRRGE
RAGEVELWSRMVRGADVLVGSRAVDGAVDVFGGVVSVDSRASVSVSRALLTEVPSVLG
VGVQEVLLAAFGLAVARWRGRGGPVVVDVEGHGRNEDAVRGADLSRTVGWFTSVYPV
RVPVESASWDEVRAGGPVVGRVVREVKETLRSLPDQGLGYGILRYLDPEHGPALARHAT
PQFGFNYLGRFTTGTDDTGDEGMTDWVPVSGPFAVGAGQDPELPVAHAVEFNAITLDTPE
GPRLGVTWSWPTTLLPESRIRELARYWDEALEGLVEHARHPEAGGLTPSDVTLVEVNQVE
LDRLQAGVAGGAEEILPVSALQEGLLFHSALASGGVDVYVGQLVFDLVGPVDVDRLRAA
VEGLVARHGVLRSGYRQLRSGEWVAVVARQVDLPWQSIDVRDGGIDGLVEEERWRRFD
MGRGPLARFVLIRTHDDRFRFVITYHHVVLDGWSVPVLLRELLALYGSSGDVSVLPGVRS
YGDFLRWVAARDAAAAEGAWRRALTGLEEPSLVAPGVSRDGVVPAAFHGAVDGDLSQK
IVAWARGRGVTVASVVQAAWALVLGRLMGRDDVVFGVTVSGRPAEVVGVEDMVGLFV
NTIPLRARLDPAESLGGFVERLQREQTELLEHQHVRLAEVQRWAGHKELFDVGMVFDNY
PVSSESPEAEFQISRTGGYNGTHYALNLVASMHGLELELEIGYRPDVFDAGRVREVWGWL
VRVLEGVVSGGGGVSVSGVDVLGVGERERLLGWGVGGPVPVVPGGGLVGLFEERVRAD
ADAVAVRGAGVVWSYGELNARVNVVARWLVGRGVGAECGVGVVMGRGVDVVVMLL
AVAKAGGFYVPVDPEWPVERVGWVLADAGVGLVVVGEGLSHVVGDFPGGEVFEFSRVV
RESCLVELVAADGVEVRNVTDGERASRLLPGHPLYVVYTSGSTGRPKGVVVTHASVGGY
LARGRDVYAGAVGGVGFVHSSLAFDLTVTVLFTPLVSGGCVVLGELDESAQGVGASFVK
VTPSHLGLLGELEGVVAGNGMLLVGGEALSGGALREWRERNPGVVVVNAYGPTELTVN
CAEFLIAPGEEVPDGPVPIGRPFAGQRMFVLDAALRVVPVGVVGELYVAGVGLARGYLG
RVGLTAERFVACPFGVPGERMYRTGDLVRWRVDGALEFVGRADDQVKVRGFRVELGEV
EGAVAAHPDVVRAVVVVREDRPGDHRLVAYVTAGGVGGDGLRSAISGLVAERLPAYMV
PSAVVVLDEIPLTPNGKVDRAALPVPEVEAGTGYRAPVSPREEVLCGLFAEVLGVERVGV
DDDFFELGGHSLLATRLISRVRAVLGVEAGVRALFEAPTVSRLERLLRERSGLGVRVPLVA
RERTGREPLSFAQQRLWFLEELEGPGAAYNIPMALRLAGVLDVEALHQALIDVIARHESLR
TLIAQDAGTAWQHILPVDDPRTRPGLPLVDIGADALQERLDEAAGRPFDLAADLPVRATV
FRLTDNDHILLLVLHHIAGDGWSMGPLARDLSTAYSARAAGAASAWRPLSVQYADYAA
WQRDVLGTEDDESSELSAQLAYWRTQLASLPAELALPTDRARPAVATYRGGRIEFTIPAD
VHRSLADLARAEGVTVFMVVQAALAALLSRLGAGDDIPIGTPIAGRTDQATEDLIGFFVNT
LVLRTDVSGDPTFAELLARVRATDLDAYAHQDIPFERLVEAVNPERSLARHPLFQVMLAF
NNAETSTPLPMAEGLAASRQDIEPGVAKFDLALYCNESRGETGDHQGIRSVFEYRRDLWD
EDTVRQLADRFLHVLAAFAAAPEQRASSVDVLRAGERDQLLHEWNDTAAALPPALLPQL
FEEQVRRTPHDVALVSGNIRLTYAELDARANRLAHLLLARGAAPETFVAVALPRTEELLV
ALLAVQKTGAGHLPLDPGFPAERLSYMLDDARPAVVLTTEDISARIPGGSHVVLDSEQVT
GELHDHPATSPAGRGNPAGPAYVIYTSGSTGQPKGVVVPSAALVNFLADMVPRLGLRGG
DRLLSVTTVGFDIAALELFVPLLSGATVVLADGETVRDPALARQTCEDHGVTMVQATPS
WWHGMLADAGDSLRGVHAVVGGEALSPGLRDALTRGARSVTNMYGPTETTIWSTSAGQ
AAGDSAPPSIGTPILNTRVYVLDAALCVVPPGVAGELYIAGDGLARGYLGRAGLTAERFV
ACPFGAPGERMYRTGDLVRWRVDGALEFVGRADDQVKVRGFRVELGEVEGAVAAHPD
VVRAVVVVREDRPGDHRLVAYVTGVDTGGLSSAVMRAVAERLPAYMVPSAVVVLDEIP
LTPNGKVDRAALPVPGVEAGAGYRAPVSPREEVLCGLFAEVLGVERVGVDDDFFGLGGH
SLLATRLISRVRAVLGVEAGVRALFEAPTVSRLERLLRERSGLGVRVPLVARERTGREPLS
FAQQRLWFLEELEGPGAAYNIPMALRLAGVLDVEALHQALIDVIARHESLRTLIARDSDGT
ARQQVLPVGDPAARPALPVVQTDADTLVAKLNEAVGRPFDLTAEMPLRATVFRVADEDH
ALLLVFHHIAGDGWSTGLLARDLSTAYAARLEGRDPQLPPLPVQYADYAAWQRDVLGTE
DDESSELSAQLAYWRTQLADLPAELALPADRVRPARASYEGGRVGFTVPAGVLRDLTRL
ARVEGVTVFMVVQAALAALLSRLGAGDDIPIGTPIAGRTDQATEDLIGFFVNTLVLRTDVS
GDPTFAELLARVRATDLDAYAHQDIPFERLVEAVNPERSLARHPLFQVMLAFDNTADGGP
VEDFPGLSAAGLPLGAGAAKFDLLFGLSEVGGELRGAVEYRCDLFDHPTAARIAERLVRV
LERVAADASVRLGELPVVSDAERACVLTEWNDTAVPGVTGTLSALFEARAAARGDAPAV
VYEGEELSYRELNTRANRLAHVLAEHGAGPERFVGVALPRSPDLVVALLAVVKSGAAYV
PLDPEYPADRLAYMAGDAAPVAVLTRGDVELPGSVPRIGLDDTEIRATLATAPGTNPGTP
VTEAHPAYMIYTSGSTGRPKGVVVSHGAIVNRLAWMQAEYRLDATDRVLQKTPAGFDVS
VWEFFWPLLEGAVLVFARPGGHRDAAYLAGLIERERITTAHFVPSMLRVFLEEPGAALCT
GLRRVICSGEALGTDLAVDFRAKLPVPLHNLYGPTEAAVDVTHHAYEPATGTATVPIGRPI
WNIRTYVLDAALRPVPPGVPGELYLAGAGLARGYHGRPALTAERFVACPFGVPGERMYR
TGDLVRWRVDGTLEFVGRADDQVKVRGFRVELGEVEGAVAAHPDVVRAVVVVREDRP
GDHRLVAYVTVGGVGGDGLRSAISGLVAERLPAYMVPSAVVVLDEIPLTPNGKVDRAGL
PVPVVSVAGFCAPSSPREEVLCGLFAEVLGVERVGVDDGFFDLGGDSILSIQLVARARRAG
LELSVRDVFEGRTVRALAAVVRGSDAGAVGVVGGAEIVLPGVGEVERWPVVEWLAERG
GGSLGGVVRGFNQSVVLAVPAGLVWEELRVLLGAVRDRHEAWRLRVLDSGALCVDGV
VPDDGSWIVRCDLSGMGVDGQVDAVRAAAVEARAWLDPSVGRVVRAVWLERGGDRSG
VLVLVAHHLVVDGVSWRVVLGDLAEGWAQVRSGGRVELGVVGTSLRGWAAALAEQG
RRGERAGEVELWSRMVRGADVLVGSRAVDGAVDVFGGVVSVDSRASVSVSRALLTEVP
SVLGVGVQEVLLAAFGLAVARWRGRGGPVVVDVEGHGRNEDAVRGADLSRTVGWFTS
VYPVRVPVESASWDEVRAGGPVVGRVVREVKETLRSLPDQGLGYGILRYLDPEHGPALA
RHATPQFGFNYLGRFTTGTDETTTADALDRAPAWSLLARSAAGQDPELPVAHAVEFNAIT
LDTPEGPRLGVTWSWPTTLLPESRIRELARYWDEALEGLVEHARHPEAGGLTPSDVGLAE
LSFAEIELLEDDWRTQG

What is claimed is:

1. A metabolically-engineered microorganism capable of synthesizing an N-acylglycine biosurfactant, comprising a chimeric fusion protein.

2. The metabolically-engineered microorganism of claim 1, wherein the chimeric fusion protein comprises a glycine adenylation domain operably linked to a condensation domain, a peptidyl carrier protein domain, a thioesterase domain, and the type II TE domain.

3. The metabolically-engineered microorganism of claim 2, wherein the glycine adenylation domain is selected from the group consisting of a DhbF protein of SEQ ID NO:10 and a PksJ protein of SEQ ID NO:12.

4. The metabolically-engineered microorganism of claim 2, wherein the glycine adenylation domain protein motif comprises a glycine adenylation domain protein motif selected from the group consisting of SEQ ID NO:40, SEQ ID NO:41, SEQ ID NO:42, SEQ ID NO:89, and SEQ ID NO:1.

5. The metabolically-engineered microorganism of claim 2, wherein the glycine adenylation domain comprises a glycine adenylation domain from the DhbF protein of SEQ ID NO:9 or the PksJ protein of SEQ ID NO:11.

6. The metabolically-engineered microorganism of claim 2, wherein the condensation domain, the peptidyl carrier protein domain, the thioesterase domain, and the type II TE domain are encoded by the surfactin gene cluster.

7. The metabolically-engineered microorganism of claim 6, wherein the surfactin gene cluster is selected from the group consisting of SrfAA-M3 (SEQ ID NO:4), SrfAA-M2 (SEQ ID NO:3), SrfAA-M1 (SEQ ID NO:2), SrfAB-M6 (SEQ ID NO:7), SrfAB-M5 (SEQ ID NO:6), SrfAB-M4 (SEQ ID NO:5), and SrfAC-M7 (SEQ ID NO:8).

8. The metabolically-engineered microorganism of claim 2, wherein the glycine adenylation domain is operably linked to the condensation domain at the amino acid sequence of SDAEKQM (SEQ ID NO: 53) or TLISDAEK (SEQ ID NO: 54), wherein E comprises the junction between the glycine adenylation domain and the condensation domain.

9. The metabolically-engineered microorganism of claim 2, wherein the glycine adenylation domain is operably linked to the peptidyl carrier protein domain at the amino acid sequence of WQEVLNVEKAGIF (SEQ ID NO: 57), wherein N comprises the junction between the glycine adenylation domain and the peptidyl carrier protein domain.

10. The metabolically-engineered microorganism of claim 2, wherein the glycine adenylation domain is operably linked to the peptidyl carrier protein domain at the amino acid sequence of RVGIDDDFFALG (SEQ ID NO: 56) or IEWDDDFFAL (SEQ ID NO: 55), wherein the third D comprises the junction between the glycine adenylation domain and the peptidyl carrier protein domain.

11. The metabolically-engineered microorganism of claim 1, wherein the microorganism is a gram (−) or a gram (+) bacteria.

12. The metabolically-engineered microorganism of claim 11, wherein the gram (+) bacteria is Bacillus subtilis.

13. The metabolically-engineered microorganism of claim 11, wherein the gram (−) bacteria Escherichia coli.

14. The metabolically-engineered microorganism of claim 1, wherein a polynucleotide encoding the chimeric fusion protein is expressed by a bacterial promoter.

15. The metabolically-engineered microorganism of claim 14, wherein the bacterial promoter comprises a PsrfA bacterial promoter.

16. The metabolically-engineered microorganism of claim 1, wherein a polynucleotide encoding the chimeric fusion protein is integrated within a genomic locus of the microorganism.

17. The metabolically-engineered microorganism of claim 16, wherein the genomic locus comprises an amyE genomic locus.

18. The metabolically-engineered microorganism of claim 16, wherein the integration comprises a homologous recombination mediated integration.

19. The metabolically-engineered microorganism of claim 1, wherein the expression of the chimeric fusion protein results in the synthesis of N-acylglycine from medium chain length β-hydroxy fatty acids.

20. The metabolically-engineered microorganism as in any of claims 1-19, in which the chimeric fusion protein is selected from the group consisting of a polynucleotide with at least 90% sequence identity to ME-B0004 (SEQ ID NO:15), ME-B0007 (SEQ ID NO:13), and ME-B0008 (SEQ ID NO:14).

21. A method for producing N-acylglycine from a microorganism, the method comprising;
a. providing a microorganism comprising a chimeric fusion protein of claim 1;
b. culturing the microorganism to produce medium chain length β-hydroxy fatty acids;
c. expressing the chimeric fusion protein, wherein the expression of the chimeric fusion protein synthesizes N-acylglycine from the medium chain length β-hydroxy fatty acids; and,
d. purifying the N-acylglycine from the microorganism to produce the N-acylglycine.

22. A method for fermenting N-acylglycine within a microorganism, the method comprising;
a. fermenting a microorganism comprising a chimeric fusion protein of claim 1;
b. expressing the chimeric fusion protein, wherein the expression of the chimeric fusion protein synthesizes N-acylglycine from a medium chain length β-hydroxy fatty acids; and,
c. fermenting N-acylglycine within the microorganism.

23. A chimeric polynucleotide sequence comprising a glycine adenylation domain.

24. The chimeric polynucleotide sequence of claim 23, wherein the glycine adenylation domain is selected from the group consisting of:
a. a polynucleotide motif comprising a glycine adenylation domain motif of SEQ ID NO:40, SEQ ID NO:41, SEQ ID NO:42, or SEQ ID NO:1;
b. a polynucleotide comprising at least 90% sequence identity to the polynucleotide of SEQ ID NO:49, or SEQ ID NO:52;
c. a polynucleotide encoding a polypeptide comprising at least 90% sequence identity to SEQ ID NO:10, SEQ ID NO:12, SEQ ID NO:58, SEQ ID NO:59, SEQ ID NO:60, SEQ ID NO:61, SEQ ID NO:62, SEQ ID NO:63, SEQ ID NO:64, SEQ ID NO:65, or SEQ ID NO:66; and,
d. a polynucleotide of a coding sequence comprising a glycine adenylation domain of Table 1.

25. The chimeric polynucleotide sequence of claim 23, the glycine adenylation domain operably linked to a condensation domain.

26. The chimeric polynucleotide sequence of claim 25, the condensation domain comprising a polynucleotide with at least 90% sequence identity to SEQ ID NO:50.

27. The chimeric polynucleotide sequence of claim 25, the condensation domain comprising a polynucleotide encoding a polypeptide with at least 90% sequence identity to SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76.

28. The chimeric polynucleotide sequence of claim 23, the glycine adenylation domain operably linked to a peptidyl carrier protein domain.

29. The chimeric polynucleotide sequence of claim 28, the peptidyl carrier protein domain comprising a polynucleotide with at least 90% sequence identity to SEQ ID NO:51.

30. The chimeric polynucleotide sequence of claim 28, the peptidyl carrier protein domain comprising a polynucleotide encoding a polypeptide with at least 90% sequence identity to SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, or SEQ ID NO:86.

31. The chimeric polynucleotide sequence of claim 23, the glycine adenylation domain operably linked to a thioesterase domain.

32. The chimeric polynucleotide sequence of claim 31, the thioesterase domain comprising a polynucleotide with at least 90% sequence identity to SEQ ID NO:51.

33. The chimeric polynucleotide sequence of claim 31, the thioesterase domain comprising a polynucleotide encoding a polypeptide with at least 90% sequence identity to SEQ ID NO:87, or SEQ ID NO:88.

34. The chimeric polynucleotide sequence of claim 23, the chimeric polynucleotide sequence comprising a NRPS fusion gene construct of a polypeptide with at least 90% sequence identity to SEQ ID NO:13, SEQ ID NO:14, or SEQ ID NO:15.

35. The chimeric polynucleotide sequence of claim 34, the expression of the NRPS fusion gene construct synthesizing N-acylglycine from a medium chain length β-hydroxy fatty acid.

36. The chimeric polynucleotide sequence of any of claims 23-35, the glycine adenylation domain transformed into a bacterial microorganism.

37. The chimeric polynucleotide sequence of claim 36, the bacterial microorganism synthesizing an N-acylglycine biosurfactant from a medium chain length β-hydroxy fatty acid.
